SEQUENCE LISTING (132 sequences)

SEQ ID NO: 1 - DNA, 2241 bp, Synthetic Construct
  misc_feature 1..2241: note = Complex-66 First Polypeptide
  source 1..2241: mol_type = other DNA
caaggacaatctggctctgtgtccaccacctgttggtgggaccctccatgcacacctaataccggcagctctggtggctctggcggaagcggaggactgtctggcagatccgatgatcacggcggaggatctgaggtgcagctggttgaatctggtggcggactggttcagcctggcggatctctgaaactgagctgtgccgccagcggcttcaccttcaacaaatacgccatgaactgggtccgacaggcccctggcaaaggccttgaatgggtcgccagaatcagaagcaagtacaacaactatgccacctactacgccgacagcgtgaaggacagattcaccatcagccgggacgacagcaagaacaccgcctacctgcagatgaacaacctgaaaaccgaggacaccgccgtgtactactgtgtgcggcacggcaacttcggcaacagctacatcagctactgggcctattggggccagggcacactggtcacagtttctagtggcggaggcggatctggcggcggtggaagtggcggcggaggttctcaaacagtggtcacccaagagcctagcctgaccgtttctcctggcggaaccgtgacactgacatgcggatcttctacaggcgccgtgaccagcggcaactaccctaattgggtgcagcagaagccaggccaggctcctagaggactgatcggcggcacaaagtttctggctcccggaacaccagccagattcagcggttctctgctcggaggaaaggccgctctgacactttctggcgtgcagcctgaggatgaggccgagtactattgcgtgctgtggtacagcaacagatgggtgttcggcggaggcaccaagctgacagttcttggaggtggcggtagccaggtccagctgaaacaatctggacccggactcgtgcagccaagccagagcctgtctatcacctgtaccgtgtccggcttcagcctgaccaattacggcgtgcactgggttcgacaatctcccggcaagggactcgaatggctgggagtgatttggagcggcggcaacaccgactacaacaccccattcaccagcagactgagcatcaacaaggacaacagcaagtcccaggtgttcttcaagatgaactccctgcagagccaggataccgccatctattactgcgctcgggccctgacctactatgactacgagtttgcctactggggacagggaaccctcgtgacagtgtctgctgctagcacaaagggccctagcgttttcccactggctcccagcagcaagtctacatccggtggaacagccgctctgggctgcctggtcaaggattactttcccgagccagtgaccgtgtcctggaatagcggagcactgacatctggcgtgcacacatttccagccgtgctgcagtctagcggcctgtactctctgtccagcgttgtgacagtgcccagcagctctctgggcacccagacctacatctgcaatgtgaaccacaagcctagcaacaccaaggtggacaagaaggtggaacccaagagctgcgataagacacacacctgtcctccatgtcctgctccagagctgctcggaggcccttccgtgtttctgttccctccaaagcctaaggacaccctgatgatcagcagaacccctgaagtgacctgcgtggtggtggatgtgtcccacgaggatcccgaagtgaagttcaattggtacgtcgacggcgtggaagtgcacaatgccaagaccaagccttgcgaggaacagtacggcagcacctacagatgcgtgtccgtgctgacagtgctgcaccaggattggctgaacggcaaagagtacaagtgcaaggtgtccaacaaggccctgcctgctcctatcgagaaaaccatcagcaaggccaagggccagcctagagaaccccaggtgtacacactgcctccaagccggaaagagatgaccaagaatcaggtgtccctgacctgcctggtcaagggcttctacccttccgatatcgccgtggaatgggagagcaatggacagcccgagaacaactacaagacaacccctcctgtgctgaagtccgacggctcattcttcctgtacagcaagctgaccgtggacaagagcagatggcagcagggcaacgtgttcagctgcagcgtgatgcacgaggccctgcacaaccactacacccagaagtccctgtctctgagccccggcaaa

SEQ ID NO: 2 - protein, 746 aa, Synthetic Construct
  REGION 1..746: note = Complex-66 First Polypeptide
QGQSGSVSTTCWWDPPCTPNTGSSGGSGGSGGLSGRSDDHGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 6 aa, Synthetic Construct
  REGION 1..6: note = Spacer
QGQSGS

SEQ ID NO: 4 - protein, 15 aa, Synthetic Construct
  REGION 1..15: note = Complex-66 Anti-CD3 MM ML15
VSTTCWWDPPCTPNT

SEQ ID NO: 5 - protein, 8 aa, Synthetic Construct
  REGION 1..8: note = CM 0011
LSGRSDDH

SEQ ID NO: 6 - protein, 249 aa, Synthetic Construct
  REGION 1..249: note = Anti-CD3 scFv
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 7 - protein, 119 aa, Synthetic Construct
  REGION 1..119: note = Complex-66/CI106 Anti-EGFR VH
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSA

SEQ ID NO: 8 - protein, 98 aa, Synthetic Construct
  REGION 1..98: note = CH1-Human IgG1-Complex-66
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 9 - protein, 98 aa, Synthetic Construct
  REGION 1..98: note = CH1-Human IgG4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 10 - protein, 14 aa, Synthetic Construct
  REGION 1..14: note = Hinge-A, Complex-66/CI106
EPKSCDKTHTCPPC

SEQ ID NO: 11 - protein, 9 aa, Synthetic Construct
  REGION 1..9: note = Hinge-B Complex-166
DKTHTCPPC

SEQ ID NO: 12-14 - skipped (3 entries)

SEQ ID NO: 15 - DNA, 783 bp, Synthetic Construct
  misc_feature 1..783: note = Complex-66/CI106 Light Chain
  source 1..783: mol_type = other DNA
caaggacaatctggacagggcctgagctgtgaaggctgggccatgaatagagagcagtgcagagctggcggcggatcttctggcggctctatctcttctggactgctgagcggcagaagcgatcaacacggcggaggctctcagatcctgctgacacagagccccgtgatcctgtctgtgtctcctggcgagagagtgtccttcagctgtagagccagccagtccatcggcaccaacatccactggtatcagcagcggaccaacggcagccccagactgctgattaagtacgccagcgagagcatcagcggcatccccagcagattttctggcagcggctctggcaccgacttcaccctgagcatcaacagcgtggaaagcgaggatatcgccgactactactgccagcagaacaacaactggcccaccacctttggagccggcaccaagctggaactgaagagaacagtggccgctcctagcgtgttcatcttcccaccttccgacgagcagctgaaaagcggcacagcctctgtcgtgtgcctgctgaacaacttctaccccagagaagccaaggtgcagtggaaggtggacaacgccctgcagagcggcaatagccaagagtctgtgaccgagcaggacagcaaggactccacctacagcctgagcagcaccctgacactgagcaaggccgactacgagaagcacaaagtgtacgcctgcgaagtgacccaccagggcctttctagccctgtgaccaagagcttcaaccggggcgagtgt

SEQ ID NO: 16 - protein, 261 aa, Synthetic Construct
  REGION 1..261: note = Complex66/CI106 Light Chain
QGQSGQGLSCEGWAMNREQCRAGGGSSGGSISSGLLSGRSDQHGGGSQILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 7 aa, Synthetic Construct
  REGION 1..7: note = Spacer - Light Chain-2nd Polypeptide
QGQSGQG

SEQ ID NO: 18 - protein, 15 aa, Synthetic Construct
  REGION 1..15: note = MM-anti-EGFR COMPLEX-66
LSCEGWAMNREQCRA

SEQ ID NO: 19 - protein, 13 aa, Synthetic Construct
  REGION 1..13: note = CM
ISSGLLSGRSDQH

SEQ ID NO: 20 - protein, 107 aa, Synthetic Construct
  REGION 1..107: note = VL-anti-EGFR COMPLEX-66/106
QILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 21 - protein, 107 aa, Synthetic Construct [sequence omitted]

SEQ ID NO: 22 - DNA, 2241 bp, Synthetic Construct
  misc_feature 1..2241: note = CI106 - 1st Polypeptide
  source 1..2241: mol_type = other DNA
caaggacaatctggatccggctatctgtggggctgcgagtggaattgtggcggcatcacaacaggctctagcggcggaagcggaggatctggtggactgtctggcagatccgatgatcatggcggcggatcccagaccgtggtcacacaagagcctagcttctccgtgtctcctggcggcacagtgaccctgacatgcagatcttctacaggcgccgtgaccaccagcaactacgccaattgggtgcagcagacccctggacaggctcctagaggactgatcggcggcaccaacaaaagagcccctggcgtcccagatagattcagcggctctatcctgggcaacaaggccgcactgacaatcacaggcgcccaggccgatgacgagagcgattactattgcgccctgtggtacagcaacctgtgggttttcggcggaggcaccaagctgacagttcttggcggaggcggaagtggtggtggcggatctggtggcggtggatctgaagtgcagctggtggaatctggcggaggacttgttcagccaggcggctctctgaagctgtcttgtgccgcctccggcttcacctttagcacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagaacaccgcctacctgcagatgaacagcctgaaaaccgaggacaccgccgtgtactactgcaccagacacggcaacttcggcaacagctatgtgtcttggtttgcctactggggccagggcacactggtcacagttagttctggcggcggaggttctcaggtgcagctgaaacagtctggccctggactggtgcagcctagccagtctctgagcatcacctgtaccgtgtccggcttctccctgaccaattacggcgtgcactgggttcgacaatccccaggcaagggactcgaatggctgggagtgatttggagcggcggcaacaccgactacaacaccccattcaccagcagactgtccatcaacaaggacaacagcaagtcccaggtgttcttcaagatgaactccctgcagagccaggataccgccatctattactgcgctcgggccctgacctactatgactacgagttcgcctattggggacagggaaccctcgtgacagtgtctgccgctagcacaaagggccctagcgttttcccactggctcccagcagcaagtctacatccggtggaacagccgctctgggctgcctggtcaaggattactttcccgagccagtgaccgtgtcctggaatagcggagcactgacatctggcgtgcacacatttccagccgtgctgcagtctagcggcctgtactctctgtccagcgttgtgacagtgcccagcagctctctgggcacccagacctacatctgcaatgtgaaccacaagcctagcaacaccaaggtggacaagaaggtggaacccaagagctgcgataagacacacacctgtcctccatgtcctgctccagagctgctcggaggcccttccgtgtttctgttccctccaaagcctaaggacaccctgatgatcagcagaacccctgaagtgacctgcgtggtggtggatgtgtcccacgaggatcccgaagtgaagttcaattggtacgtcgacggcgtggaagtgcacaatgccaagaccaagccttgcgaggaacagtacggcagcacctacagatgcgtgtccgtgctgacagtgctgcaccaggattggctgaacggcaaagagtacaagtgcaaggtgtccaacaaggccctgcctgctcctatcgagaaaaccatcagcaaggccaagggccagcctagagaaccccaggtgtacacactgcctccaagccggaaagagatgaccaagaatcaggtgtccctgacctgcctggtcaagggcttctacccttccgatatcgccgtggaatgggagagcaatggacagcccgagaacaactacaagacaacccctcctgtgctgaagtccgacggctcattcttcctgtacagcaagctgaccgtggacaagagcagatggcagcagggcaacgtgttcagctgcagcgtgatgcacgaggccctgcacaaccactacacccagaagtccctgtctctgagccccggcaaa

SEQ ID NO: 23 - protein, 747 aa, Synthetic Construct
  REGION 1..747: note = CI106- 1st Polypeptide
QGQSGSGYLWGCEWNCGGITTGSSGGSGGSGGLSGRSDDHGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 24 - protein, 15 aa, Synthetic Construct
  REGION 1..15: note = CI106 - Anti-CD3 MM
GYLWGCEWNCGGITT

SEQ ID NO: 25 - protein, 249 aa, Synthetic Construct
  REGION 1..249: note = CI106 - Anti-CD3 scFv
QTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSS

SEQ ID NO: 26 - DNA, 2241 bp, Synthetic Construct
  misc_feature 1..2241: note = CI106 Heavy Chain -
  source 1..2241: mol_type = other DNA
caaggccagtctggatccggttatctgtggggttgcgagtggaattgcggagggatcactacaggctcgagcggtggcagcggtggctctggtggtctgagcggccgttccgatgatcatggcggcggttctcaaactgtagtaactcaagaaccaagcttctccgtctcccctgggggaacagtcacacttacctgccgaagtagtacaggtgctgttacgaccagtaactatgccaattgggtacaacaaacgcctggtcaggctccgcgcggattgataggaggcacgaataaacgggcacccggtgtcccggacagattcagcggaagcatactcggtaataaggcagctcttactatcactggggcccaagctgatgatgaaagtgattattattgtgcgctctggtacagcaacctctgggtgtttgggggtggcacgaaacttactgtcttgggcggcggcggatcagggggaggtggctctggaggaggaggctcagaagtccaactggtcgaatccgggggagggctcgtacagccgggtgggtccctcaaactctcttgtgcggcctcagggtttaccttcagtacatacgcgatgaattgggtccggcaggccagtgggaaagggctcgaatgggtaggacgaatccgatcaaaatacaacaactacgctacttattacgctgattccgtgaaggacagattcacaatatcccgcgacgatagcaagaatacggcatatcttcagatgaattctcttaaaactgaggataccgctgtgtattactgcacaagacatggtaattttggaaactcatatgtctcttggttcgcttattggggacagggcacgttggttaccgtgtctagcggaggtggtggatcccaggtgcagctgaaacagagcggcccgggcctggtgcagccgagccagagcctgagcattacctgcaccgtgagcggctttagcctgaccaactatggcgtgcattgggtgcgccagagcccgggcaaaggcctggaatggctgggcgtgatttggagcggcggcaacaccgattataacaccccgtttaccagccgcctgagcattaacaaagataacagcaaaagccaggtgttttttaaaatgaacagcctgcaaagccaggataccgcgatttattattgcgcgcgcgcgctgacctattatgattatgaatttgcgtattggggccagggcaccctggtgaccgtgagcgcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaatttgaagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcctcaatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 27 - protein, 747 aa, Synthetic Construct
  REGION 1..747: note = CI106 - Heavy Chain
QGQSGSGYLWGCEWNCGGITTGSSGGSGGSGGLSGRSDDHGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 217 aa, Synthetic Construct
  REGION 1..217: note = Fc without C-terminal lysine
PAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 330 aa, Synthetic Construct
  REGION 1..330: note = CI106 - CH1-Hinge-Fc
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 108 aa, Synthetic Construct
  REGION 1..108: note = I2C Anti-CD3 VL1
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTV

SEQ ID NO: 31 - protein, 125 aa, Synthetic Construct
  REGION 1..125: note = I2C Anti-CD3 VH1
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 32 - protein, 109 aa, Synthetic Construct
  REGION 1..109: note = v16 Anti-CD3 VL1
QTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVL

SEQ ID NO: 33 - protein, 125 aa, Synthetic Construct
  REGION 1..125: note = V16 Anti-CD3 VH1
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSS

SEQ ID NO: 34 - protein, 11 aa, Synthetic Construct
  REGION 1..11: note = EGFR VL CDR1
RASQSIGTNIH

SEQ ID NO: 35 - protein, 7 aa, Synthetic Construct
  REGION 1..7: note = EGFR VL CDR2
YASESIS

SEQ ID NO: 36 - protein, 9 aa, Synthetic Construct
  REGION 1..9: note = EGFR VL CDR3
QQNNNWPTT

SEQ ID NO: 37 - protein, 5 aa, Synthetic Construct
  REGION 1..5: note = EGFR VH CDR1
NYGVH

SEQ ID NO: 38 - protein, 16 aa, Synthetic Construct
  REGION 1..16: note = EGFR VH CDR2
VIWSGGNTDYNTPFTS

SEQ ID NO: 39 - protein, 11 aa, Synthetic Construct
  REGION 1..11: note = EGFR VH CDR3
ALTYYDYEFAY

SEQ ID NO: 40 - protein, 14 aa, Synthetic Construct
  REGION 1..14: note = CD3 I2C VL CDR1
SSTGAVTSGNYPNG

SEQ ID NO: 41 - protein, 7 aa, Synthetic Construct
  REGION 1..7: note = CD3 I2C VL CDR2
GTKFLAP

SEQ ID NO: 42 - protein, 9 aa, Synthetic Construct
  REGION 1..9: note = CD3 I2C VL CDR3
VLWYSNRWV

SEQ ID NO: 43 - protein, 5 aa, Synthetic Construct
  REGION 1..5: note = CD3 I2C VH CDR1
KYAMN

SEQ ID NO: 44 - protein, 19 aa, Synthetic Construct
  REGION 1..19: note = CD3 I2C VH CDR2
RIRSKYNNYATYYADSVKD

SEQ ID NO: 45 - protein, 14 aa, Synthetic Construct
  REGION 1..14: note = CD3 I2C VH CDR3
HGNFGNSYISYWAY

SEQ ID NO: 46 - protein, 14 aa, Synthetic Construct
  REGION 1..14: note = CD3 v16 VL CDR1
RSSTGAVTTSNYAN

SEQ ID NO: 47 - protein, 7 aa, Synthetic Construct
  REGION 1..7: note = CD3 v16 VL CDR2
GTNKRAP

SEQ ID NO: 48 - protein, 9 aa, Synthetic Construct
  REGION 1..9: note = CD3 v16 VL CDR3
ALWYSNLWV

SEQ ID NO: 49 - protein, 5 aa, Synthetic Construct
  REGION 1..5: note = CD3 v16 VH CDR1
TYAMN

SEQ ID NO: 50 - protein, 19 aa, Synthetic Construct
  REGION 1..19: note = CD3 v16 VH CDR2
RIRSKYNNYATYYADSVKD

SEQ ID NO: 51 - protein, 14 aa, Synthetic Construct
  REGION 1..14: note = CD3 v16 VH CDR3
HGNFGNSYVSWFAY

SEQ ID NO: 52 - protein, 15 aa, Synthetic Construct
  REGION 1..15: note = CD3 MM
MMYCGGNEVLCGPRV

SEQ ID NO: 53 - protein, 15 aa, Synthetic Construct
  REGION 1..15: note = CD3 MM
GYRWGCEWNCGGITT

SEQ ID NO: 54 - protein, 15 aa, Synthetic Construct
  REGION 1..15: note = CD3 MM
MMYCGGNEIFCEPRG

SEQ ID NO: 55 - protein, 15 aa, Synthetic Construct
  REGION 1..15: note = CD3 MM
GYGWGCEWNCGGSSP

SEQ ID NO: 56 - protein, 15 aa, Synthetic Construct
  REGION 1..15: note = CD3 MM
MMYCGGNEIFCGPRG

SEQ ID NO: 57 - protein, 8 aa, Synthetic Construct
  REGION 1..8: note = CM
LSGRSDNH

SEQ ID NO: 58 - protein, 12 aa, Synthetic Construct
  REGION 1..12: note = CM
TSTSGRSANPRG

SEQ ID NO: 59 - protein, 10 aa, Synthetic Construct
  REGION 1..10: note = CM
VHMPLGFLGP

SEQ ID NO: 60 - protein, 8 aa, Synthetic Construct
  REGION 1..8: note = CM
AVGLLAPP

SEQ ID NO: 61 - protein, 8 aa, Synthetic Construct
  REGION 1..8: note = CM
QNQALRMA

SEQ ID NO: 62 - protein, 8 aa, Synthetic Construct
  REGION 1..8: note = CM
ISSGLLSS

SEQ ID NO: 63 - protein, 13 aa, Synthetic Construct
  REGION 1..13: note = CM
ISSGLLSGRSDNH

SEQ ID NO: 64 - protein, 8 aa, Synthetic Construct
  REGION 1..8: note = CM
LSGRSGNH

SEQ ID NO: 65 - protein, 8 aa, Synthetic Construct
  REGION 1..8: note = CM
LSGRSDIH

SEQ ID NO: 66 - protein, 8 aa, Synthetic Construct
  REGION 1..8: note = CM
LSGRSDQH

SEQ ID NO: 67 - protein, 8 aa, Synthetic Construct
  REGION 1..8: note = CM
LSGRSDTH

SEQ ID NO: 68 - protein, 8 aa, Synthetic Construct
  REGION 1..8: note = CM
LSGRSDYH

SEQ ID NO: 69 - protein, 8 aa, Synthetic Construct
  REGION 1..8: note = CM
LSGRSDNP

SEQ ID NO: 70 - protein, 8 aa, Synthetic Construct
  REGION 1..8: note = CM
LSGRSANP

SEQ ID NO: 71 - protein, 8 aa, Synthetic Construct
  REGION 1..8: note = CM
LSGRSANI

SEQ ID NO: 72 - protein, 8 aa, Synthetic Construct
  REGION 1..8: note = CM
LSGRSDNI

SEQ ID NO: 73 - protein, 15 aa, Synthetic Construct
  REGION 1..15: note = CM
ISSGLLSGRSANPRG

SEQ ID NO: 74 - protein, 18 aa, Synthetic Construct
  REGION 1..18: note = CM
AVGLLAPPTSGRSANPRG

SEQ ID NO: 75 - protein, 17 aa, Synthetic Construct
  REGION 1..17: note = CM
AVGLLAPPSGRSANPRG

SEQ ID NO: 76 - protein, 13 aa, Synthetic Construct
  REGION 1..13: note = CM
ISSGLLSGRSDDH

SEQ ID NO: 77 - protein, 13 aa, Synthetic Construct
  REGION 1..13: note = CM
ISSGLLSGRSDIH

SEQ ID NO: 78 - protein, 13 aa, Synthetic Construct
  REGION 1..13: note = CM
ISSGLLSGRSDTH

SEQ ID NO: 79 - protein, 13 aa, Synthetic Construct
  REGION 1..13: note = CM
ISSGLLSGRSDYH

SEQ ID NO: 80 - protein, 13 aa, Synthetic Construct
  REGION 1..13: note = CM
ISSGLLSGRSDNP

SEQ ID NO: 81 - protein, 13 aa, Synthetic Construct
  REGION 1..13: note = CM
ISSGLLSGRSANP

SEQ ID NO: 82 - protein, 13 aa, Synthetic Construct
  REGION 1..13: note = CM
ISSGLLSGRSANI

SEQ ID NO: 83 - protein, 18 aa, Synthetic Construct
  REGION 1..18: note = CM
AVGLLAPPGGLSGRSDDH

SEQ ID NO: 84 - protein, 18 aa, Synthetic Construct
  REGION 1..18: note = CM
AVGLLAPPGGLSGRSDIH

SEQ ID NO: 85 - protein, 18 aa, Synthetic Construct
  REGION 1..18: note = CM
AVGLLAPPGGLSGRSDQH

SEQ ID NO: 86 - protein, 18 aa, Synthetic Construct
  REGION 1..18: note = CM
AVGLLAPPGGLSGRSDTH

SEQ ID NO: 87 - protein, 18 aa, Synthetic Construct
  REGION 1..18: note = CM
AVGLLAPPGGLSGRSDYH

SEQ ID NO: 88 - protein, 18 aa, Synthetic Construct
  REGION 1..18: note = CM
AVGLLAPPGGLSGRSDNP

SEQ ID NO: 89 - protein, 18 aa, Synthetic Construct
  REGION 1..18: note = CM
AVGLLAPPGGLSGRSANP

SEQ ID NO: 90 - protein, 18 aa, Synthetic Construct
  REGION 1..18: note = CM
AVGLLAPPGGLSGRSANI

SEQ ID NO: 91 - protein, 13 aa, Synthetic Construct
  REGION 1..13: note = CM
ISSGLLSGRSDNI

SEQ ID NO: 92 - protein, 18 aa, Synthetic Construct
  REGION 1..18: note = CM
AVGLLAPPGGLSGRSDNI

SEQ ID NO: 93 - protein, 13 aa, Synthetic Construct
  REGION 1..13: note = CM
ISSGLLSGRSGNH

SEQ ID NO: 94 - protein, 4 aa, Synthetic Construct
  REGION 1..4: note = Linker
GGGS

SEQ ID NO: 95 - protein, 5 aa, Synthetic Construct
  REGION 1..5: note = Linker
GSGGS

SEQ ID NO: 96 - protein, 4 aa, Synthetic Construct
  REGION 1..4: note = Linker
GGSG

SEQ ID NO: 97 - protein, 5 aa, Synthetic Construct
  REGION 1..5: note = Linker
GGSGG

SEQ ID NO: 98 - protein, 5 aa, Synthetic Construct
  REGION 1..5: note = Linker
GSGSG

SEQ ID NO: 99 - protein, 5 aa, Synthetic Construct
  REGION 1..5: note = Linker
GSGGG

SEQ ID NO: 100 - protein, 5 aa, Synthetic Construct
  REGION 1..5: note = Linker
GGGSG

SEQ ID NO: 101 - protein, 5 aa, Synthetic Construct
  REGION 1..5: note = Linker
GSSSG

SEQ ID NO: 102 - protein, 17 aa, Synthetic Construct
  REGION 1..17: note = Linker
GGGGSGGGGSGGGGSGS

SEQ ID NO: 103 - protein, 7 aa, Synthetic Construct
  REGION 1..7: note = Linker
GGGGSGS

SEQ ID NO: 104 - protein, 15 aa, Synthetic Construct
  REGION 1..15: note = Linker
GGGGSGGGGSGGGGS

SEQ ID NO: 105 - protein, 20 aa, Synthetic Construct
  REGION 1..20: note = Linker
GGGGSGGGGSGGGGSGGGGS

SEQ ID NO: 106 - protein, 5 aa, Synthetic Construct
  REGION 1..5: note = Linker
GGGGS

SEQ ID NO: 107 - protein, 10 aa, Synthetic Construct
  REGION 1..10: note = Linker
GGGGSGGGGS

SEQ ID NO: 108 - protein, 8 aa, Synthetic Construct
  REGION 1..8: note = Linker
GGGSGGGS

SEQ ID NO: 109 - protein, 12 aa, Synthetic Construct
  REGION 1..12: note = Linker
GGGSGGGSGGGS

SEQ ID NO: 110 - protein, 11 aa, Synthetic Construct
  REGION 1..11: note = Linker
GSSGGSGGSGG

SEQ ID NO: 111 - protein, 24 aa, Synthetic Construct
  REGION 1..24: note = Linker
GGGSGGGGSGGGGSGGGGSGGGGS

SEQ ID NO: 112 - protein, 16 aa, Synthetic Construct
  REGION 1..16: note = Linker
GSTSGSGKPGSSEGST

SEQ ID NO: 113 - protein, 17 aa, Synthetic Construct
  REGION 1..17: note = Linker
SKYGPPCPPCPAPEFLG

SEQ ID NO: 114 - protein, 12 aa, Synthetic Construct
  REGION 1..12: note = Linker
GGSLDPKGGGGS

SEQ ID NO: 115 - protein, 20 aa, Synthetic Construct
  REGION 1..20: note = Linker
PKSCDKTHTCPPCPAPELLG

SEQ ID NO: 116 - protein, 12 aa, Synthetic Construct
  REGION 1..12: note = Linker
GKSSGSGSESKS

SEQ ID NO: 117 - protein, 14 aa, Synthetic Construct
  REGION 1..14: note = Linker
GSTSGSGKSSEGKG

SEQ ID NO: 118 - protein, 18 aa, Synthetic Construct
  REGION 1..18: note = Linker
GSTSGSGKSSEGSGSTKG

SEQ ID NO: 119 - protein, 18 aa, Synthetic Construct
  REGION 1..18: note = Linker
GSTSGSGKPGSGEGSTKG

SEQ ID NO: 120 - protein, 740 aa, Synthetic Construct
  REGION 1..740: note = Complex-66-1st polypeptide without spacer and without C-terminal lysine
VSTTCWWDPPCTPNTGSSGGSGGSGGLSGRSDDHGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 121 - protein, 254 aa, Synthetic Construct
  REGION 1..254: note = Complex-66-2nd polypeptide without spacer
LSCEGWAMNREQCRAGGGSSGGSISSGLLSGRSDQHGGGSQILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 741 aa, Synthetic Construct
  REGION 1..741: note = CI106-1st polypeptide without spacer
GYLWGCEWNCGGITTGSSGGSGGSGGLSGRSDDHGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 123 - protein, 8 aa, Synthetic Construct
  REGION 1..8: note = Linker
GGGSSGGS

SEQ ID NO: 124 - protein, 5 aa, Synthetic Construct
  REGION 1..5: note = Linker; note = the entire sequence of amino acids can be repeated one or more times
GGGGS

SEQ ID NO: 125 - protein, 10 aa, Synthetic Construct
  REGION 1..10: note = Linker
GSSGGSGGSG

SEQ ID NO: 126 - DNA, 2238 bp, Synthetic Construct
  misc_feature 1..2238: note = Complex-66 Polynucleotide encoding 1st Polypeptide
  source 1..2238: mol_type = other DNA
caaggacaatctggctctgtgtccaccacctgttggtgggaccctccatgcacacctaataccggcagctctggtggctctggcggaagcggaggactgtctggcagatccgatgatcacggcggaggatctgaggtgcagctggttgaatctggtggcggactggttcagcctggcggatctctgaaactgagctgtgccgccagcggcttcaccttcaacaaatacgccatgaactgggtccgacaggcccctggcaaaggccttgaatgggtcgccagaatcagaagcaagtacaacaactatgccacctactacgccgacagcgtgaaggacagattcaccatcagccgggacgacagcaagaacaccgcctacctgcagatgaacaacctgaaaaccgaggacaccgccgtgtactactgtgtgcggcacggcaacttcggcaacagctacatcagctactgggcctattggggccagggcacactggtcacagtttctagtggcggaggcggatctggcggcggtggaagtggcggcggaggttctcaaacagtggtcacccaagagcctagcctgaccgtttctcctggcggaaccgtgacactgacatgcggatcttctacaggcgccgtgaccagcggcaactaccctaattgggtgcagcagaagccaggccaggctcctagaggactgatcggcggcacaaagtttctggctcccggaacaccagccagattcagcggttctctgctcggaggaaaggccgctctgacactttctggcgtgcagcctgaggatgaggccgagtactattgcgtgctgtggtacagcaacagatgggtgttcggcggaggcaccaagctgacagttcttggaggtggcggtagccaggtccagctgaaacaatctggacccggactcgtgcagccaagccagagcctgtctatcacctgtaccgtgtccggcttcagcctgaccaattacggcgtgcactgggttcgacaatctcccggcaagggactcgaatggctgggagtgatttggagcggcggcaacaccgactacaacaccccattcaccagcagactgagcatcaacaaggacaacagcaagtcccaggtgttcttcaagatgaactccctgcagagccaggataccgccatctattactgcgctcgggccctgacctactatgactacgagtttgcctactggggacagggaaccctcgtgacagtgtctgctgctagcacaaagggccctagcgttttcccactggctcccagcagcaagtctacatccggtggaacagccgctctgggctgcctggtcaaggattactttcccgagccagtgaccgtgtcctggaatagcggagcactgacatctggcgtgcacacatttccagccgtgctgcagtctagcggcctgtactctctgtccagcgttgtgacagtgcccagcagctctctgggcacccagacctacatctgcaatgtgaaccacaagcctagcaacaccaaggtggacaagaaggtggaacccaagagctgcgataagacacacacctgtcctccatgtcctgctccagagctgctcggaggcccttccgtgtttctgttccctccaaagcctaaggacaccctgatgatcagcagaacccctgaagtgacctgcgtggtggtggatgtgtcccacgaggatcccgaagtgaagttcaattggtacgtcgacggcgtggaagtgcacaatgccaagaccaagccttgcgaggaacagtacggcagcacctacagatgcgtgtccgtgctgacagtgctgcaccaggattggctgaacggcaaagagtacaagtgcaaggtgtccaacaaggccctgcctgctcctatcgagaaaaccatcagcaaggccaagggccagcctagagaaccccaggtgtacacactgcctccaagccggaaagagatgaccaagaatcaggtgtccctgacctgcctggtcaagggcttctacccttccgatatcgccgtggaatgggagagcaatggacagcccgagaacaactacaagacaacccctcctgtgctgaagtccgacggctcattcttcctgtacagcaagctgaccgtggacaagagcagatggcagcagggcaacgtgttcagctgcagcgtgatgcacgaggccctgcacaaccactacacccagaagtccctgtctctgagccccggc

SEQ ID NO: 127 - protein, 747 aa, Synthetic Construct
  REGION 1..747: note = Complex-66 1st Polypeptide with spacer and C-terminal lysine
QGQSGSVSTTCWWDPPCTPNTGSSGGSGGSGGLSGRSDDHGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 741 aa, Synthetic Construct
  REGION 1..741: note = Complex-66-1st polypeptide without spacer and with C-terminal lysine
VSTTCWWDPPCTPNTGSSGGSGGSGGLSGRSDDHGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 7 aa, Synthetic Construct
  REGION 1..7: note = CM
ALAHGLF

SEQ ID NO: 130 - protein, 11 aa, Synthetic Construct
  REGION 1..11: note = CM
APRSALAHGLF

SEQ ID NO: 131 - protein, 12 aa, Synthetic Construct
  REGION 1..12: note = CM
ISSGLLSGRSNI

SEQ ID NO: 132 - protein, 7 aa, Synthetic Construct
  REGION 1..7: note = CM
LSGRSNI

(19) United States
(12) Patent Application Publication
(10) Pub. No.: US 2025/0243283 A1
(43) Pub. Date: Jul. 31, 2025

(54) ACTIVATABLE ANTI-CD3, ANTI-EGFR, HETEROMULTIMERIC BISPECIFIC POLYPEPTIDE COMPLEX

(71) Applicant: CytomX Therapeutics, Inc., South San Francisco, CA (US)

(72) Inventor: Sayantan MITRA, Mountain View, CA (US)

(21) Appl. No.: 18/701,356

(22) PCT Filed: Oct. 17, 2022
(86) PCT No.: PCT/US2022/078218

§ 371 Date: Apr. 15, 2024

Related U.S. Application Data
(60) Provisional application No. 63/256,405, filed on Oct. 15, 2021.

(51) Int. Cl. C07K 16/28; A61K 39/00; A61P 35/00

(52) U.S. Cl. CPC C07K 16/2863; A61P 35/00; C07K 16/2809; A61K 2039/505; C07K 2317/31; C07K 2317/52; C07K 2317/565; C07K 2317/622; C07K 2317/73

(57) ABSTRACT

The present disclosure relates to activatable anti-EGFR, anti-CD3, heteromultimeric bispecific polypeptide complexes (HBPCs) and methods of making and using the same.

### CROSS-REFERENCE TO RELATED APPLICATIONS

This application claims the priority benefit of U.S. Provisional Application Nos. 63/256,405, filed Oct. 15, 2021, and 63/370,894, filed Aug. 9, 2022, which are incorporated herein by reference in their entireties.

### REFERENCE TO SEQUENCE LISTING SUBMITTED ELECTRONICALLY VIA EFS WEB

The content of the electronically submitted sequence listing (4680_001PC02_Seqlisting_ST26.xml; Size: 178,813 bytes; and Date of Creation: Oct. 17, 2022) submitted in this application is incorporated herein by reference in its entirety.

### FIELD

The present disclosure relates to activatable anti-EGFR, anti-CD3, activatable heteromultimeric bispecific polypeptide complexes (HBPCs) and methods of making and using the same.

### BACKGROUND

Tumor antigen-specific T cells can recognize tumor antigens through their native receptors and are involved in immune-mediated anti-tumor activity. This requires multiple T-cell co-stimulatory receptors and T-cell negative regulators, or co-inhibitory receptors, acting in concert to control T-cell activation, proliferation, and gain or loss of effector function. However, tumor-specific T-cell responses are difficult to mount and sustain in cancer patients, due to the numerous immune escape mechanisms of tumor cells. However, attempts have been made to harness T cells for cancer therapies. Such approaches include using T cell engaging bispecific antibodies which bind a surface target antigen on a cancer cell and a T cell surface antigen, such as CD3, on T cells. Generally, by binding each target, T cell engaging bispecifics hold T cells in close physical proximity with a cancer cell and allow for cytotoxic T cell proteins and enzymes to attack tumor cells and cause apoptosis, thereby killing cancer cells.

Epidermal growth factor receptor (EGFR) is a receptor and transmembrane glycoprotein that exhibits intrinsic tyrosine kinase activity regulates numerous cellular processes including, but not limited to, activation of signal transduction pathways that control cell proliferation, differentiation, cell survival, apoptosis, angiogenesis, mitogenesis, and metastasis (Atalay et al., Ann. Oncology 14:1346-1363 (2003); Tsao and Herbst, Signal 4:4-9 (2003); Herbst and Shin, Cancer 94:1593-1611 (2002); Modjtahedi et al., Br. J. Cancer 73:228-235 (1996)). Overexpression of EGFR is associated with numerous human cancers, including cancers of the bladder, brain, head and neck, pancreas, lung, breast, ovary, colon, prostate, and kidney. EGFR is also expressed in the cells of normal tissues at lower levels than expressed in malignant cells.

Bispecific antibodies that engage EGFR and CD3 suffer from drawbacks, including T cell mediated toxicity (i.e., cytokine release) and EGFR-related toxicities due to off-tumor binding. Additionally, manufacturing challenges arise due to the complex structure of bispecific antibodies and high levels of aggregation during manufacturing and scale-up. Accordingly, there is a need for immunotherapeutic options which have an improved safety profile, as well as improved manufacturability.

### BRIEF SUMMARY

Provided herein is an activatable anti-EGFR, anti-CD3 heteromultimeric bispecific polypeptide complex (HBPC) comprising: (a) a first polypeptide comprising (i) a first single-chain variable fragment (scFv) comprising a first heavy chain variable domain (VH1) and a first light chain variable domain (VL1) that together form a first CD3-targeting domain that specifically binds a first CD3 polypeptide, (ii) a first masking moiety (MM1), (iii) a first cleavable moiety (CM1), (iv) a second heavy chain variable domain (VH2), and (v) a first monomeric Fc domain (Fc1); (b) a second polypeptide comprising (i) a second light chain variable domain (VL2), wherein VL2 and VH2 together form a first EGFR-targeting domain that specifically binds a first EGFR, (ii) a second masking moiety (MM2), and (iii) a second cleavable moiety (CM2); (c) a third polypeptide comprising (i) a second scFv comprising a third heavy chain variable domain (VH3) and a third light chain variable domain (VL3) that together form a second CD3-targeting domain that specifically binds a second CD3 polypeptide, (ii) a third masking moiety (MM3), (iii) a third cleavable moiety (CM3); (iv) a fourth heavy chain variable domain (VH4), and (v) a second monomeric Fc domain (Fc2); and (d) a fourth polypeptide comprising (i) a fourth light chain variable domain (VL4), wherein VL4 and VH2 together form a second EGFR-targeting domain that specifically binds EGFR, (ii) a fourth masking moiety (MM4), and (iii) a fourth cleavable moiety (CM4), wherein: (1) the first polypeptide comprises the amino acid sequence of SEQ ID NO: 120, (2) the second polypeptide comprises the amino acid sequence of SEQ ID NO:121, (3) the third polypeptide comprises the amino acid sequence of SEQ ID NO:120, and (4) the fourth polypeptide comprises the amino acid sequence of SEQ ID NO:121.

In some aspects of the activatable anti-EGFR, anti-CD3 heteromultimeric bispecific polypeptide complex (HBPC) provided herein, (1) the first polypeptide comprises the amino acid sequence of SEQ ID NO:2, (2) the second polypeptide comprises the amino acid sequence of SEQ ID NO:16, (3) the third polypeptide comprises the amino acid sequence of SEQ ID NO:2, and (4) the second polypeptide comprises the amino acid sequence of SEQ ID NO:16.

In some aspects, the first and third polypeptides comprise the amino acid sequence of SEQ ID NO:128. In some aspects, the first and third polypeptides comprise the amino acid sequence of SEQ ID NO:127.

Also provided herein is a pharmaceutical composition comprising the activatable bispecific polypeptide complex described herein and a pharmaceutically acceptable carrier.

Also provided herein are kits comprising the pharmaceutical composition.

Also provided herein are nucleic acids comprising nucleotide sequences that encode the first polypeptide, the second polypeptide, the third polypeptide, and the fourth polypeptide of the activatable bispecific polypeptide complex described herein. Also provided herein are vectors comprising the nucleic acids provided herein and host cell comprising the vectors provided herein.

Also provided herein is a method of producing an activatable anti-EGFR, anti-CD3 heteromultimeric bispecific polypeptide complex (HBPC) comprising: (a) culturing the host cell described herein in a liquid culture medium under conditions sufficient to produce the activatable HBPC; and (b) recovering the activatable HBPC.

Also provided herein are methods of treating a disease in a subject comprising administering a therapeutically effective amount of the activatable HBPC or the pharmaceutical composition of comprising an activatable HBPC to the subject. In some aspects, the subject is a human. In some aspects, the disease is a cancer.

In some aspects, the activatable HBPC described herein or the pharmaceutical composition of described herein is for use in inhibiting tumor growth in a subject in need thereof.

In some aspects, the activatable HBPC described herein or the pharmaceutical composition of described herein is for the manufacture of a medicament for treating cancer.

### BRIEF DESCRIPTION OF THE FIGURES

FIG. 1 is a schematic of an activatable anti-EGFR, anti-CD3 heteromultimeric bispecific polypeptide complex (HBPC) described herein.

FIG. 2A shows binding to EGFR by activated CI106 (control), activated Complex-66 (HBPC), CI106, and Complex-66.

FIG. 2B shows binding to CD3 by activated CI106, activated Complex-66 (HBPC), CI106 (control), and Complex-66.

FIG. 3 shows the cytotoxicity of HT29 cells following treatment with activated CI106, activated Complex-66, CI106, and Complex-66 (HBPC).

FIG. 4 shows the tumor volume in a HT29-luc2 xenograft tumor model as a function of time following treatment with CI106 (control) and Complex-66.

FIG. 5 shows the tumor volume in a HCT116 xenograft tumor model as a function of time following treatment with activated Complex-66 and Complex-66.

FIG. 6A shows cross-reactivity of activatable CI106 (control), Complex-66, and activated Complex-66 with cynomolgus monkey EGFR.

FIG. 6B shows cross-reactivity of Complex-66 and activated Complex-66 with cynomolgus monkey CD3.

FIG. 7 shows functional cytotoxic activity against target cells expressing cynomolgus EGFR and using cynomolgus effector cells following treatment with activated Complex-66 and Complex-66.

FIG. 8 is a graph of % monomer vs. concentration for CI106 (control) and Complex-66.

FIGS. 9A-9C show the flow cytometry assessment of CI107 binding to EGFR and CD3 expressed on the surface of HT29 cells (A), HCT116 cells (B), or Jurkat cells (C). The apparent K_{d} was calculated from duplicate experiments in HT29 cells and triplicate experiments in Jurkat cells.

FIGS. 10A-10C show the percent cytotoxicity mediated by CI107 in HCT116-Luc2 cells (A, C) and HT29-Luc2 cells (B, D). After 48 hours of culture, HCT116-Luc2 or HT29-Luc2 cell viability and cytotoxicity were measured relative to untreated controls (A, B). After 16 hours of culture, CD69 expression was measured by flow cytometry. MFI, mean fluorescence intensity (C, D).

FIGS. 11A-11E show cytokine release following treatment with CI107, measured after 16 hours of culture. (A) IFN-γ, (B) IL-2, (C) IL-6, (D) MCP-1, and (E) TNF-α.

FIGS. 12A-12B shows the tumor volume after treatment with test TCBs in mice harboring HT29-Luc2 tumors and engrafted with human PBMCs. (A) Mice were treated once weekly for 3 weeks with vehicle (PBS) or 0.3 mg/kg CI020, CI011, CI040, or CI048 (n=8 per group). Tumor volume was measured twice weekly. (B) NSG mice harboring HT29-Luc2 tumors and engrafted with human PBMCs were treated with vehicle or 1 mg/kg of CI020, CI011, CI040, or CI048. Tumors were harvested 7 days after dosing, and immunohistochemistry for CD3 was performed. Dark staining indicates CD3+ cells.

FIGS. 13A-13B show tumor volumes following treatment with CI107 once weekly for 3 weeks in HT29 (A) and HCT116 (B) xenograft tumors. Tumor volume was measured twice weekly. * p<0.5; ** p<0.01; **** p<0.0001.

FIGS. 14A-14B show levels of IL-6 (A) and IFN-γ (B) measured 8 hours after dosing with CI107.

FIG. 14C shows levels of aspartate aminotransferase (AST) measured by serum chemistry analysis 48 hours after dosing with CI107 (C).

FIG. 14D shows plasma concentrations of Act-CI107 and CI107 measured by ELISA using anti-idiotype capture and anti-human Fc detection. CI107 lines represent data from 3 individual animals dosed with 2.0 mg/kg CI107; Act-TCB lines represent single animals dosed with 0.06 mg/kg or 0.18 mg/kg Act-TCB.

### DETAILED DESCRIPTION

In order that the present disclosure may be more readily understood, certain terms are first defined. As used in this application, except as otherwise expressly provided herein, each of the following terms shall have the meaning set forth below. Additional definitions are set forth throughout the application.

### Definitions

As used herein, the term “heteromultimeric bispecific polypeptide complex” and “HBPC” are used interchangeably to refer to a set of polypeptides that together form a complex that has binding domains that are capable of binding to two different biological targets.

The term “activatable” when used in connection with the term “heteromultimeric bispecific polypeptide complex” or “HBPC” refers herein to an HBPC whose binding activity is impaired by the presence of masking moieties appended to the structure of the HBPC. The terms “activated” and “act-” can each be used to refer to an activated HBPC. The terms “activated” and “unmasked,” are used interchangeably herein.

As used herein, the term “EGFR” refers to a receptor and transmembrane glycoprotein and a member of the protein kinase superfamily. Human epidermal growth factor receptor is a 170 kDa transmembrane receptor encoded by the c-erb B-1 protooncogene, and exhibits intrinsic tyrosine kinase activity (Modjtahedi et al., Br. J. Cancer 73:228-235 (1996); Herbst and Shin, Cancer 94:1593-1611 (2002)). There are also known isoforms and variants of EGFR (e.g., alternative RNA transcripts, truncated versions, polymorphisms, etc.), which are contemplated for use herein. EGFR regulates numerous cellular processes via tyrosine-kinase mediated signal transduction pathways, including, but not limited to, activation of signal transduction pathways that control cell proliferation, differentiation, cell survival, apoptosis, angiogenesis, mitogenesis, and metastasis (Atalay et al., Ann. Oncology 14:1346-1363 (2003); Tsao and Herbst, Signal 4:4-9 (2003); Herbst and Shin, Cancer 94:1593-1611 (2002); Modjtahedi et al., Br. J. Cancer 73:228-235 (1996)). Overexpression of EGFR is associated with numerous human cancers, including cancers of the bladder, brain, head and neck, pancreas, lung, breast, ovary, colon, prostate, and kidney. EGFR is also expressed in the cells of normal tissues at lower levels than expressed in malignant cells. Exemplary anti-EGFR antigen-binding proteins include but are not limited to human wildtype EGFR (NCBI Accession No. NG_007726.E), human wildtype EGFR Transcript Variant 1 (NCBI Accession No. NP_005219.2), human wildtype EGFR Transcript Variant 2 (NCBI Accession No. NP_958439.1), human wildtype EGFR Transcript Variant 3 (NCBI Accession No. NP_958440.1), human wildtype EGFR Transcript Variant 4 (NCBI Accession No. NP_958441.1), human wildtype EGFR Transcript Variant 5 (NCBI Accession No. NP_001333826.1), human wildtype EGFR Transcript Variant 6 (NCBI Accession No. NP_001333827.1), human wildtype EGFR Transcript Variant 7 (NCBI Accession No. NP_001333828.1), human wildtype EGFR Transcript Variant 8 (NCBI Accession No. NM_001346941.2), human wildtype EGFR Transcript Variant EGFRvIII (NCBI Accession No. NP_001333870.1), and the like.

The term “CD3” or “cluster of differentiation 3” as used herein refers to a protein complex of six chains which are subunits of the T cell receptor complex. (Janeway et al., p. 166, 9^{th} ed.) The TCR α:β heterodimer associates with CD3 subunits to complete the TCR cell-surface antigen receptor. Two CD3ε chains, a CD3γ chain, and a CD3δ chain and a homodimer of CD3ξ chains complete the T cell receptor complex, which is involved in the recognition of peptides bound to the major histocompatibility complex class I and II and involves T cell activation. The CD3 antigen is expressed by mature T lymphocytes and by a subset of thymocytes. The CD3-targeting domain that specifically binds a CD3 polypeptide, disclosed herein, can be from any vertebrate source, including mammals such as primates (e.g. humans) and rodents (e.g., mice and rats). The term encompasses “full-length,” unprocessed CD3 (e.g., unprocessed or unmodified CD38 or CD3 γ) as well as any form of CD3 that results from processing in the cell. The term also encompasses naturally occurring variants of CD3, including, for example, splice variants or allelic variants. An anti-CD3 antigen-binding domain described herein can specifically bind to human wildtype CD3E (NCBI Accession No. NM_000733.3).

The term “T cell,” as used herein is defined as a thymus-derived lymphocyte that participates in a variety of cell-mediated immune reactions. The term “regulatory T cell” as used herein refers to a CD4+CD25+FoxP3+ T cell. “Treg” is the abbreviation used herein for a regulatory T cell.

The term “helper T cell” as used herein refers to a CD4+ T cell; helper T cells recognize antigen bound to MHC Class II molecules. There are at least two types of helper T cells, Th1 and Th2, which produce different cytokines. Helper T cells become CD25+when activated, but only transiently become FoxP3+.

The term “cytotoxic T cell” as used herein refers to a CD8+ T cell; cytotoxic T cells recognize antigen bound to MHC Class I molecules.

The term “variable region” or “variable domain” refers to the domain of an antigen binding protein (e.g., an antibody) heavy or light chain that is involved in binding the antigen binding protein (e.g., antibody) to antigen. The variable regions or domains of the heavy chain and light chain (VH and VL, respectively) of an antigen binding protein such as an antibody can be further subdivided into regions of hypervariability (or hypervariable regions, which may be hypervariable in sequence and/or form of structurally defined loops), such as hypervariable regions (HVRs) or complementarity-determining regions (CDRs), interspersed with regions that are more conserved, termed framework regions (FRs). In general, there are three HVRs (HVR-H1, HVR-H2, HVR-H3) or CDRs (CDR-H1, CDR-H2, CDR-H3) in each heavy chain variable region, and three HVRs (HVR-L1, HVR-L2, HVR-L3) or CDRs in (CDR-L1, CDR-L2, CDR-L3) in each light chain variable region. “Framework regions” and “FR” are known in the art to refer to the non-HVR or non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). Within each VH and VL, three HVRs or CDRs and four FRs are typically arranged from amino-terminus to carboxy-terminus in the following order: FR1, HVR1, FR2, HVR2, FR3, HVR3, FR4 in the case of HVRs, or FR1, CDR1, FR2, CDR2, FR3, CDR3, FR4 in the case of CDRs (See also Chothia and Lesk J. Mot. Biol., 195, 901-917 (1987)). A single VH or VL domain can be sufficient to confer antigen-binding specificity. In addition, antibodies that bind a particular antigen can be isolated using a VH or VL domain from an antibody that binds the antigen to screen a library of complementary VL or VH domains, respectively. See, e.g., Portolano et al. J. Immunol. 150:880-887 (1993); Clarkson et al., Nature 352:624-628 (1991).

The term “heavy chain variable region” (VH) as used herein refers to a region comprising heavy chain HVR-H1, FR-H2, HVR-H2, FR-H3, and HVR-H3. For example, a heavy chain variable region may comprise heavy chain CDR-H1, FR-H2, CDR-H2, FR-H3, and CDR-H3. In some aspects, a heavy chain variable region also comprises at least a portion of an FR-H1 and/or at least a portion of an FR-H4.

The term “heavy chain constant region” as used herein refers to a region comprising at least three heavy chain constant domains, C_{H}1, C_{H}2, and C_{H}3. Nonlimiting exemplary heavy chain constant regions include γ, δ, and α. Nonlimiting exemplary heavy chain constant regions also include ε and μ.

The term “light chain variable region” (VL) as used herein refers to a region comprising light chain HVR-L1, FR-L2, HVR-L2, FR-L3, and HVR-L3. In some aspects, the light chain variable region comprises light chain CDR-L1, FR-L2, CDR-L2, FR-L3, and CDR-L3. In some aspects, a light chain variable region also comprises an FR-L1 and/or an FR-L4.

The term “light chain constant region” as used herein refers to a region comprising a light chain constant domain, CL. Nonlimiting exemplary light chain constant regions include 2 and K.

The term “light chain” (LC) as used herein refers to a polypeptide comprising at least a light chain variable region, with or without a leader sequence. In some aspects, a light chain comprises at least a portion of a light chain constant region. The term “full-length light chain” as used herein refers to a polypeptide comprising a light chain variable region and a light chain constant region, with or without a leader sequence.

The term “antibody” refers to an immunoglobulin molecule or an immunologically active portion of an immunoglobulin (Ig) molecule, i.e., a molecule that contains an antigen binding site that specifically binds (immunoreacts with) an antigen. An “antigen-binding portion” of an antibody or polypeptide (also called an “antigen-binding fragment”) refers to one or more portions of an antibody or polypeptide that bind specifically to the target antigen. Antibodies and antigen-binding portions include, but are not limited to, polyclonal, monoclonal, chimeric, domain antibody, single chain antibodies, Fab, and F(ab′)₂ fragments, scFvs, Fd fragments, Fv fragments, single domain antibody (sdAb) fragments, dual-affinity re-targeting antibodies (DARTs), dual variable domain immunoglobulins; isolated complementarity determining regions (CDRs), and a combination of two or more isolated CDRs, which can optionally be joined by a synthetic linker, and a Fab expression library. A nonhuman antibody, e.g., a camelid antibody, may be humanized by recombinant methods to reduce its immunogenicity in a human.

The CDR sequences specified herein are determined in accordance with the Kabat numbering system (i.e., the “Kabat CDRs”) as described in Abhinandan, K. R. and Martin, A.C.R. (2008) “Analysis and improvements to Kabat and structurally correct numbering of antibody variable domains”, Molecular Immunology, 45, 3832-3839, which is incorporated herein by reference in its entirety. The Kabat CDRs are defined as CDR-L1: residues L24-L34; CDR-L2: residues L50-L56; CDR-L3: residues L89-L97; CDR-H1: residues H31-H35; CDR-H2: residues H50-H65; and CDR-H3: residues H95-H102, where “L” refers to the light chain variable domain and “H” refers to the heavy chain variable domain.

“Specifically binds” or “immunospecifically binds” means that the targeting domain, antibody or antigen-binding fragment reacts with one or more antigenic determinants of the desired antigen and does not react with other polypeptides or binds at much lower affinity (Kd>10-6), wherein a smaller Kd represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (kₒₙ) and the “off rate constant” (k_{off}) can be determined by calculation of the concentrations and the actual rates of association and dissociation. (See Nature 361:186-87 (1993)). The ratio of k_{off}/kₒₙ enables the cancellation of all parameters not related to affinity, and is equal to the dissociation constant K_{d}. (See, generally, Davies et al. (1990) Annual Rev Biochem 59:439-473). In some aspects, the antigen-targeting domain, antibody, or antigen-binding fragment that specifically binds to its corresponding antigen exhibits a Kd of less than about 10 μM, and in some aspects, less than about 100 μM with respect to the target antigen.

An immunoglobulin may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes.

An “anti-antigen” antibody or polypeptide refers to an antibody or polypeptide that binds specifically to the antigen. For example, an anti-CD3 polypeptide binds specifically to CD3.

As used herein, the terms “MM” and “masking moiety” are used interchangeably to refer to a peptide that interferes with binding of the antigen-binding domain to its corresponding target. For example, MM1 refers to a peptide that interferes with binding of a first CD3-targeting domain to a first CD3 polypeptide; MM2 refers to a peptide that interferes with binding of a first EGFR-targeting domain to a first EGFR (target); MM3 refers to a peptide that interferes with binding of a second CD3-targeting domain to a second CD3 polypeptide; and MM4 refers to a peptide that interferes with binding of a second EGFR-targeting domain to a second EGFR (target). The extent to which a masking moiety interferes with the binding of the antigen-binding domain to its corresponding target is quantified by its “masking efficiency.” The terms “masking efficiency” and “ME” are used interchangeably herein to refer to a ratio that is determined as follows:

ME=EC50, activatable HBPC (i.e., not cleaved by protease)
EC50, activated HBPC

As used herein, the terms “CM” and “cleavable moiety” are used interchangeably to refer to a peptide substrate that is susceptible to cleavage by a protease that is upregulated in tumor cells. Protease-mediated cleavage of the CM results in the release of the MM from the structure of the activatable HBPC, thereby generating an “activated” (i.e., unmasked) product, where each corresponding “activated” (i.e., unmasked) first and/or second antigen-binding domain is free to bind its respective target.

The term “isolated polynucleotide” as used herein refers to a recombinant polynucleotide or polynucleotide of synthetic origin which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence. Polynucleotides in accordance with the invention include the nucleic acid molecules encoding the first, second, and third polypeptides.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

As discussed herein, minor variations in the amino acid sequences described herein (i.e., each reference sequence) are contemplated as being encompassed by the present disclosure, provided that the resulting analog sequence maintains at least 75%, more preferably at least 80%, 90%, 95%, and most preferably 99% sequence identity to the reference sequence. In particular, conservative amino acid replacements are contemplated. Conservative replacements are those that take place within a family of amino acids that are related with respect to the nature of their side chains. Amino acids may be divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, within the heteromultimeric bispecific polypeptide complex described herein, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a CDR or framework region. Whether an amino acid change results in a functional polypeptide complex can readily be determined by assaying the specific activity of the resulting molecule, i.e., the resulting analog sequence. Assays are described in detail herein. Preferred amino- and carboxy-termini of analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the invention.

A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991).

Exemplary amino acid substitutions also include those which: (1) reduce susceptibility to proteolysis in regions of the activatable anti-EGFR, anti-CD3 heteromultimeric bispecific polypeptide other than in the cleavable linker comprising the CM, (2) reduce susceptibility to oxidation, (3) alter binding affinity for forming protein complexes, (4) alter binding affinities to antigen, and (4) confer or modify other physicochemical or functional properties of such analogs. Such amino acid substitutions may be identified using known mutagenesis methods and/or directed molecular evolution methods using the assays described herein. See, e.g., WO 2001/032712, U.S. Pat. No. 7,432,083, U.S. Pub. No. 2004/0180340, and U.S. Pat. No. 6,297,053, each of which is incorporated herein by reference. Analogs may be prepared by introducing one or more mutations in a reference sequence within an (activatable) heteromultimeric bispecific polypeptide complex. For example, single or multiple amino acid substitutions may be made in the reference sequence (preferably in the portion of the polypeptide outside the domain(s) forming intermolecular contacts).

As used herein, by “pharmaceutically acceptable” or “pharmacologically compatible” is meant a material that is not biologically or otherwise undesirable, e.g., the material may be incorporated into a pharmaceutical composition administered to an individual or subject without causing any significant undesirable biological effects or interacting in a deleterious manner with any of the other components of the composition in which it is contained. Pharmaceutically acceptable carriers or excipients have for example met the required standards of toxicological and manufacturing testing and/or are included on the Inactive Ingredient Guide prepared by the U.S. Food and Drug administration.

A “patient” as used herein includes any patient who is afflicted with a cancer. The terms “subject” and “patient” are used interchangeably herein.

The terms “cancer,” “cancerous,” or “malignant” refer to or describe the physiological condition in mammals that is typically characterized by unregulated cell growth. Examples of cancer include, for example, melanoma, such as unresectable or metastatic melanoma, leukemia, lymphoma, blastoma, carcinoma and sarcoma. More particular examples of such cancers include chronic myeloid leukemia, acute lymphoblastic leukemia, Philadelphia chromosome positive acute lymphoblastic leukemia (Ph+ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia (AML), and chronic lymphocytic leukemia (CML).

The term “tumor” as used herein refers to any mass of tissue that results from excessive cell growth or proliferation, either benign (non-cancerous) or malignant (cancerous), including pre-cancerous lesions.

“Administering” refers to the physical introduction of a composition comprising a therapeutic agent to a subject, using any of the various methods and delivery systems known to those skilled in the art. Routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, in some embodiments, orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

“Treatment” or “therapy” of a subject refers to any type of intervention or process performed on, or the administration of an active agent to, the subject with the objective of reversing, alleviating, ameliorating, inhibiting, slowing down progression, development, severity or recurrence of a symptom, complication or condition, or biochemical indicia associated with a disease.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. A beneficial effect can also take the form of arresting, slowing, retarding, or stabilizing deleterious progression of a marker of a solid tumor. Effective treatment may refer to alleviation of at least one symptom of a solid tumor. Such effective treatment may, e.g., reduce patient pain, reduce the size and/or number of lesions, may reduce or prevent metastasis of a tumor, and/or may slow tumor growth.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to delay other unwanted cell proliferation. In some embodiments, an effective amount is an amount sufficient to prevent or delay tumor recurrence. An effective amount can be administered in one or more administrations. The effective amount of the drug or composition may: (i) reduce the number of cancer cells; (ii) reduce tumor size; (iii) inhibit, retard, slow to some extent and may stop cancer cell infiltration into peripheral organs; (iv) inhibit, slow to some extent and may stop tumor metastasis; (v) inhibit tumor growth; (vi) prevent or delay occurrence and/or recurrence of tumor; and/or (vii) relieve to some extent one or more of the symptoms associated with the cancer.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver, spleen, and/or bone marrow (including antibodies, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The use of the alternative (e.g., “or”) should be understood to mean either one, both, or any combination thereof of the alternatives. As used herein, the indefinite articles “a” or “an” should be understood to refer to “one or more” of any recited or enumerated component.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% or 20% (i.e., +10% or +20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” should be assumed to be within an acceptable error range for that particular value or composition.

As described herein, any concentration range, percentage range, ratio range or integer range is to be understood to include the value of any integer within the recited range and, when appropriate, fractions thereof (such as one-tenth and one-hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary of Biochemistry and Molecular Biology, 2006, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

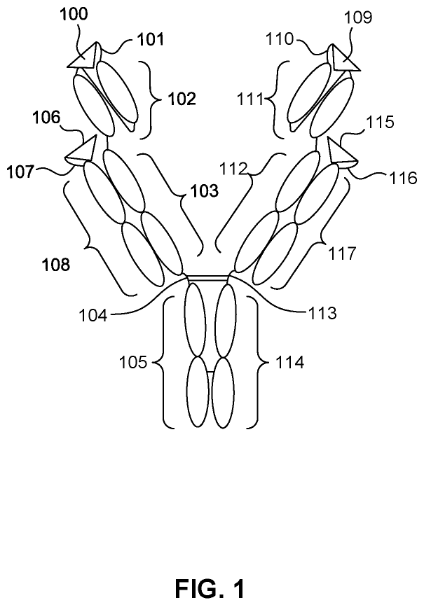

Schematic representations of activatable polypeptides of the present disclosure, e.g., FIG. 1, are not intended to be exclusive. Other sequence elements, such as linkers, spacers, and signal sequences, may be present before, after, or between the listed sequence elements in such schematic representations. It is also to be appreciated that a MM and a CM can be joined to a VH of an antibody or polypeptide instead of to a VL of an antibody or polypeptide, and vice versa.

Various aspects of the disclosure are described in further detail in the following subsections.

### Activatable Anti-EGFR, Anti-CD3 Heteromultimeric Bispecific Polypeptide Complex

The present disclosure provides an activatable anti-EGFR, anti-CD3 heteromultimeric bispecific polypeptide complex (HBPC) comprising: (a) a first polypeptide comprising (i) a first single-chain variable fragment (scFv) comprising a first heavy chain variable domain (VH1) and a first light chain variable domain (VL1) that together form a first cluster of differentiation (CD3)-targeting domain that specifically binds a first CD3 polypeptide; (ii) a first masking moiety (MM1); (iii) a first cleavable moiety (CM1); (iv) a second heavy chain variable domain (VH2), (v) and a first monomeric Fc domain (Fc1); (b) a second polypeptide comprising (i) a light chain variable domain (VL2), wherein VL2 and VH2 together form a first EGFR-binding domain that specifically binds a first EGFR target, (ii) a second masking moiety (MM2), and (iii) a second cleavable moiety (CM2); (c) a third polypeptide comprising (i) a second scFv comprising a third heavy chain variable domain (VH3) and a third light chain variable domain (VL3) that together form a second CD3-targeting domain that specifically binds a second CD3 polypeptide, (ii) a third masking moiety (MM3), (iii) a third cleavable moiety (CM3), (iv) fourth heavy chain variable domain (VH4), and (v) a second monomeric Fc domain (Fc2); and (d) a fourth polypeptide comprising (i) a fourth light chain variable domain (VL4), wherein VL4 and VH4 together form a second EGFR-targeting domain that specifically binds EGFR, (ii) a fourth masking moiety (MM4), and (iii) a fourth cleavable moiety (CM4). In some aspects, the first CD3 polypeptide target and the second CD3 polypeptide target are the same and/or the first EGFR target and the second EGFR target are the same. In certain aspects, the pair of VL1 and VH1 is the same as the pair of VL3 and VH3 and/or the pair of VL2 and VH2 is the same as the pair of VL4 and VH4.

In a certain aspects, the activatable anti-EGFR, anti-CD3 heteromultimeric bispecific polypeptide complex (HBPC) comprises: (1) a first polypeptide comprising the amino acid sequence of SEQ ID NO:120, (2) a second polypeptide comprising the amino acid sequence of SEQ ID NO:121, (3) a third polypeptide comprising the amino acid sequence of SEQ ID NO:120, and (4) a fourth polypeptide comprising the amino acid sequence of SEQ ID NO: 121.

In a certain aspect, present disclosure provides an activatable anti-EGFR, anti-CD3 heteromultimeric bispecific polypeptide complex (HBPC) comprising (1) a first polypeptide comprising the amino acid sequence of SEQ ID NO:2, (2) a second polypeptide comprising the amino acid sequence of SEQ ID NO:16, (3) a third polypeptide comprising the amino acid sequence of SEQ ID NO:2, and (4) a second polypeptide comprising the amino acid sequence of SEQ ID NO:16.

In a certain aspect, present disclosure provides an activatable anti-EGFR, anti-CD3 heteromultimeric bispecific polypeptide complex (HBPC) wherein the first and third polypeptides comprise the amino acid sequence of SEQ ID NO:128. In a certain aspect, present disclosure provides an activatable anti-EGFR, anti-CD3 heteromultimeric bispecific polypeptide complex (HBPC) wherein the first and third polypeptides comprise the amino acid sequence of SEQ ID NO: 127.

As demonstrated in the Examples herein, the activatable HBPC of the present invention provides a compound that exhibits relatively low binding activity to EGFR and CD3. When activated, however, by exposure to one or more proteases, the binding properties of the HBPC component of the activatable HBPC were successfully restored, yielding a relatively potent anti-CD3, anti-EGFR heteromultimeric bispecific polypeptide complex. The activatable HBPC of the present invention exhibited other advantageous properties as compared to activatable or masked molecules known in the art, including improved anti-tumor activity, improved aggregation resistance, as well as relatively low concentration-dependent aggregation. The latter property is particularly beneficial during purification where relatively high localized concentrations of activatable HBPC product may be generated.

In some aspects, the present disclosure provides an activatable anti-EGFR, anti-CD3 heteromultimeric bispecific polypeptide complex comprising: (a) a first polypeptide comprising (i) a first single-chain variable fragment (scFv) comprising a first heavy chain variable domain (VH1) and a first light chain variable domain (VL1) that together form a first CD3-targeting domain that specifically binds a CD3 polypeptide, (ii) a first masking moiety (MM1), and (iii) a first cleavable moiety (CM1); a second heavy chain variable domain (VH2), (iv) a first monomeric Fc domain (Fc1); wherein VH1 comprises a VH CDR1, VH CDR2, and VH CDR3 comprising: (i) a VH CDR1 comprising the amino acid sequence KYAMN (SEQ ID NO:43), (ii) a VH CDR2 comprising the amino acid sequence RIRSKYNNYATYYADSVKD (SEQ ID NO:44), and (iii) a VH CDR3 comprising the amino acid sequence HGNFGNSYISYWAY (SEQ ID NO:45); and wherein VL1 comprises a VL CDR1, VL CDR2, and VL CDR3 comprising: (i) a VL CDR1 comprising the amino acid sequence SSTGAVTSGNYPNG (SEQ ID NO:40), (ii) a VL CDR2 comprising the amino acid sequence GTKFLAP (SEQ ID NO:41), and (iii) a VH CDR3 comprising the amino acid sequence VLWYSNRWV (SEQ ID NO:42).

In some aspects, the activatable HBPC further comprises (b) a second polypeptide comprising (i) a second light chain variable domain (VL2) that specifically binds EGFR when paired with the first polypeptide EGFR-targeting heavy chain variable domain (VH2), (ii) a second masking moiety (MM2), and (iii) a second cleavable moiety (CM2); (c) a third polypeptide comprising (i) a second scFv comprising a third heavy chain variable domain (VH3) and a third light chain variable domain (VL3), (ii) a third masking moiety (MM3), (iii) a third cleavable moiety (CM3); (iv) a heavy chain variable domain (VH4), and (v) a second monomeric Fc domain (Fc2); and (d) a fourth polypeptide comprising (i) a fourth light chain variable domain (VL4), wherein VL4 and VH4 together form an EGFR-targeting domain that specifically binds EGFR, (ii) a fourth masking moiety (MM4), and (iii) a fourth cleavable moiety (CM4).

In some aspects, the CD3 polypeptide is the epsilon chain of CD3. In some aspects, the first scFv (anti-CD3 scFv) (comprising VH1) and VL1) and/or the second scFv (comprising VH3 and VL3) comprises the amino acid sequence of SEQ ID NO:6. In some aspects, the first scFv and the second scFv each comprise the amino acid sequence of SEQ ID NO:6.

In some aspects of the present disclosure, the (first polypeptide) VH1 and the (third polypeptide) VH3 each comprise: (i) a VH CDR1 comprising the amino acid sequence KYAMN (SEQ ID NO:43), (ii) a VH CDR2 comprising the amino acid sequence RIRSKYNNYATYYADSVKD (SEQ ID NO:44), and (iii) a VH CDR3 comprising the amino acid sequence HGNFGNSYISYWAY (SEQ ID NO:45); and a VL1 comprising (i) a VL CDR1 comprising the amino acid sequence SSTGAVTSGNYPNG (SEQ ID NO:40), (ii) a VL CDR2 comprising the amino acid sequence GTKFLAP (SEQ ID NO: 41), and (iii) a VL CDR3 comprising the amino acid sequence VLWYSNRWV (SEQ ID NO:42). In some aspects of the present disclosure, VH1 and VH3 each comprise a heavy chain variable domain at least 90% identical, at least 91%, at least 92%, at least 93%, at least 94%, at least 95%, at least 96%, at least 97%, at least 98%, or at least 99% identical to SEQ ID NO:31. In some aspects of the present disclosure, the VL1 and VL3 each comprise a light chain variable domain at least 90% identical, at least 91%, at least 92%, at least 93%, at least 94%, at least 95%, at least 96%, at least 97%, at least 98%, or at least 99% identical to SEQ ID NO:30.

In some aspects of the present disclosure, the first and third polypeptides comprise a VH1 and VH3 comprising the amino acid sequences of SEQ ID NO:31 and a VL1 and VL3 comprising the amino acid sequence of SEQ ID NO:30.

In some aspects of the present disclosure, the (first polypeptide) VH2 and the (third polypeptide) VH4 each comprise: (i) a VH CDR1 comprising the amino acid sequence NYGVH (SEQ ID NO:37), (ii) a VH CDR2 comprising the amino acid sequence VIWSGGNTDYNTPFTS (SEQ ID NO:38), and (iii) a VH CDR3 comprising the amino acid sequence ALTYYDYEFAY (SEQ ID NO:39).

In some aspects of the present disclosure, the (first polypeptide) VH2 and the (third polypeptide) VH4 comprise a heavy chain variable domain at least 90% identical, at least 91%, at least 92%, at least 93%, at least 94%, at least 95%, at least 96%, at least 97%, at least 98%, or at least 99% identical to SEQ ID NO:7

In some aspects of the present disclosure, the (first polypeptide) VH2 and the (third polypeptide) VH4 each comprise: (a) (i) a VH CDR1 comprising the amino acid sequence NYGVH (SEQ ID NO:37), (ii) a VH CDR2 comprising the amino acid sequence VIWSGGNTDYNTPFTS (SEQ ID NO:38), (iii) a VH CDR3 comprising the amino acid sequence ALTYYDYEFAY (SEQ ID NO:39), and (b) an amino acid sequence that is at least 90% identical, at least 91%, at least 92%, at least 93%, at least 94%, at least 95%, at least 96%, at least 97%, at least 98%, or at least 99% identical to SEQ ID NO:7.

In some aspects of the present disclosure, the (first polypeptide) VH2 and the (third polypeptide) VH4 comprise the amino acid sequence of SEQ ID NO:7.

In some aspects activatable anti-EGFR, anti-CD3 heteromultimeric bispecific polypeptide complex described herein, the first polypeptide further comprises a structural arrangement from amino-terminus to carboxy-terminus of:

MM1-CM1-scFv-VH2-CH1-hinge region-Fc1, wherein each “-” is independently a direct or indirect (e.g., via a linker) linkage.

In some aspects of the activatable anti-EGFR, anti-CD3 heteromultimeric bispecific polypeptide complex described herein, the third polypeptide comprises a structural arrangement from amino-terminus to carboxy-terminus of: MM3-CM3-scFv-VH4-CH1-hinge region-Fc2, wherein each “-” is independently a direct or indirect (e.g., via a linker) linkage.

In some aspects of the activatable anti-EGFR, anti-CD3 heteromultimeric bispecific polypeptide complex described herein, the first and third polypeptides further comprise one or more optional linkers, which are described herein below in more detail.

In some aspects of the activatable anti-EGFR, anti-CD3 heteromultimeric bispecific polypeptide complex disclosed herein, the VL2 comprises (i) a VL CDR1 comprising the amino acid sequence RASQSIGTNIH (SEQ ID NO:34), (ii) a VL CDR2 comprising the amino acid sequence YASESIS (SEQ ID NO:35), and (iii) a VL CDR3 comprising the amino acid sequence QQNNNWPTT (SEQ ID NO:36). In some aspects, the VL4 comprises a (i) VL CDR1 comprising the amino acid sequence RASQSIGTNIH (SEQ ID NO:34), (ii) a VL CDR2 comprising the amino acid sequence YASESIS (SEQ ID NO: 35), and (iii) a VL CDR3 comprising the amino acid sequence QQNNNWPTT (SEQ ID NO:36).

In some aspects of the activatable anti-EGFR, anti-CD3 heteromultimeric bispecific polypeptide complex disclosed herein, the VL2 comprises the amino acid sequence of SEQ ID NO:20. In some aspects of the activatable anti-EGFR, anti-CD3 heteromultimeric bispecific polypeptide complex disclosed herein, the VL4 comprises the amino acid sequence of SEQ ID NO:20.

In some aspects, the present disclosure provides a VL2 comprising: (a) (i) a CDR1 comprising the amino acid sequence RASQSIGTNIH (SEQ ID NO:34), (ii) a CDR2 comprising the amino acid sequence YASESIS (SEQ ID NO:35), (iii) a CDR3 comprising the amino acid sequence QQNNNWPTT (SEQ ID NO:36), and (b) an amino acid sequence that is at least 90% identical, at least 91%, at least 92%, at least 93%, at least 94%, at least 95%, at least 96%, at least 97%, at least 98%, or at least 99% identical to SEQ ID NO:20.

In some aspects of the activatable HBPC described herein, the second polypeptide can comprise a structural arrangement from amino-terminus to carboxy-terminus of: MM2-CM2-VL2, wherein each “-” is independently a direct or indirect (e.g., via a linker) linkage.

In some aspects of the activatable HBPC described herein, the fourth polypeptide can comprise a structural arrangement from amino-terminus to carboxy-terminus of: MM4-CM4-VL4, wherein each “-” is independently a direct or indirect (e.g., via a linker) linkage.

In some aspects of the activatable HBPC described herein, the second and fourth polypeptides comprises one or more linkers, which are described herein below in more detail.

In some aspects of the activatable anti-EGFR, anti-CD3 heteromultimeric bispecific polypeptide described herein, the first polypeptide monomeric Fc domain (Fc1) binds to the third polypeptide monomeric Fc domain (Fc2). In some aspects of the activatable anti-EGFR, anti-CD3 heteromultimeric bispecific polypeptide described herein, the first polypeptide and the third polypeptide further comprise an immunoglobulin hinge region. In some aspects, the first polypeptide and third polypeptide hinge regions comprise the same sequence. Suitable Fc regions are described further below herein.

As provided above, in some aspects, the second and fourth polypeptides can comprise any of the linkers discussed above herein.

The activatable anti-EGFR, anti-CD3 HBPC provided herein comprises a masking moiety (MM). As used herein, the term “masking moiety” and “MM”, are used interchangeably herein to refer to a peptide that, when positioned proximal to a targeting domain, interferes with binding of the targeting domain to its target. In some aspects, the MM is an amino acid sequence that is coupled, or otherwise attached, to the activatable anti-EGFR, anti-CD3 HBPC and is attached to the activatable anti-EGFR, anti-CD3 HBPC such that the MM reduces the ability of the activatable anti-EGFR, anti-CD3 HBPC to specifically bind to its targets. In some aspects, MM1 and MM3 prevent or decrease the ability of the activatable anti-EGFR, anti-CD3 HBPC from specifically binding to CD3. In some aspects, MM2 and MM4 prevent or decreases the ability of the activatable anti-EGFR, anti-CD3 HBPC from specifically binding to EGFR. In some aspects, the MM binds specifically to the antigen targeting domain(s). Suitable MMs can be identified using any of a variety of known techniques.

For example, anti-EGFR masking moieties that are suitable for use in the practice of the present disclosure include any that are known in the art, including those described in, for example, PCT Publication Nos. WO 2013/163631, WO 2015/013671, WO 2016/014974, WO 2019/075405, and WO 2019/213444, each of which are incorporated herein by reference in their entireties. Anti-CD3 masking moieties that are suitable for use in the practice of the present disclosure include any of those that are known in the art, including those described in, for example, PCT Publication Nos. WO2013/163631, WO 2015/013671, WO 2016/014974, WO 2019/075405, and WO 2019/213444, each of which is incorporated herein by reference in their entireties.

In some aspects of the activatable anti-EGFR, anti-CD3 HBPC provided herein, the MM1, the MM2, the MM3, and/or the MM4 each independently comprises from 5 amino acids to about 40 amino acids, or any range therebetween, and including both 5 amino acids and 40 amino acids. As used herein, the term “MM1” and “MM3” indicate a masking moiety on the CD3 targeting domain. As used herein, the term “MM2” and “MM4” indicate a masking moiety on the EGFR targeting domain.

In some aspects of the activatable anti-EGFR, anti-CD3 heteromultimeric bispecific polypeptide provided herein, MM1 comprises the amino acid sequence of SEQ ID NO: 4. In some aspects of the activatable anti-EGFR, anti-CD3 heteromultimeric bispecific polypeptide provided herein, MM2 comprises the amino acid sequence of SEQ ID NO: 18. In some aspects of the activatable anti-EGFR, anti-CD3 heteromultimeric bispecific polypeptide provided herein, MM3 comprises the amino acid sequence of SEQ ID NO: 4. In some aspects of the activatable anti-EGFR, anti-CD3 heteromultimeric bispecific polypeptide provided herein, MM4 comprises the amino acid sequence of SEQ ID NO: 18.

The activatable anti-EGFR, anti-CD3 heteromultimeric bispecific polypeptide complex (HBPCs) of the disclosure are activated when the cleavable moiety is cleaved by a protease, thereby generating an activated (i.e., unmasked) anti-EGFR, anti-CD3 heteromultimeric bispecific polypeptide complex (HBPC) that is capable of binding to EGFR and CD3. By comparison, the activatable anti-EGFR, anti-CD3 heteromultimeric bispecific polypeptide complexes (HBPCs) exhibit greatly reduced binding to EGFR and CD3 compared to the activated heteromultimeric bispecific polypeptide because the activatable HBPC remains masked until activated by proteases in the tumor environment. Without wishing to by bound by theory or mechanism, the typical protease expression levels in healthy tissues are likely due to the presence of endogenous inhibitors and/or unfavorable protease pH conditions, while protease activity is generally up-regulated within the tumor environment through up-regulation of protease expression, activation of zymogen, down-modulation of inhibitor expression, or a combination of these effects (See Desnoyers et al., ScienceTranslationalMedicine.org, vol. 5, Issue 207 (October 2013), hereby incorporated by reference).

These activatable anti-EGFR, anti-CD3 heteromultimeric bispecific polypeptide complexes (HBPCs) can therefore be useful in the treatment of a subject having cancer, where proteolytic activity in the tumor microenvironment is upregulated relative to normal tissue and controlled in normal tissues. The greatly reduced binding to EGFR and CD3 of the activatable HBPCs in normal tissue may allow for a reduction in the side effects associated with anti-EGFR and anti-CD3 engagement outside the tumor. In some aspects, the activatable anti-EGFR, anti-CD3 heteromultimeric bispecific polypeptide comprises a first, second, third, and/or fourth CM (CM1, CM2, CM3 and/or CM4, respectively).

In some aspects, the CM can be specific for a particular protease, which is useful in leveraging the dysregulated protease activity in tumor cells for targeted anti-EGFR, anti-CD3 heteromultimeric bispecific polypeptide activation at the site of treatment and/or diagnosis. There are reports in the literature of increased levels of proteases in a number of cancers, e.g., liquid tumors or solid tumors. See, e.g., La Rocca et al, (2004) British J. of Cancer 90 (7): 1414-1421. Numerous studies have demonstrated the correlation of aberrant protease levels, e.g., uPA, legumain, MT-SP1, matrix metalloproteases (MMPs), in solid tumors. (See e.g., Murthy R V, et al. “Legumain expression in relation to clinicopathologic and biological variables in colorectal cancer,” Clin Cancer Res. 11 (2005): 2293-2299; Nielsen B S, et al. “Urokinase plasminogen activator is localized in stromal cells in ductal breast cancer,” Lab Invest 81 (2001): 1485-1501; Look O R, et al. “In situ localization of gelatinolytic activity in the extracellular matrix of metastases of colon cancer in rat liver using quenched fluorogenic DQ-gelatin,” J Histochem Cytochem. 51 (2003): 821-829). A CM can serve as a substrate for multiple proteases, e.g. a substrate for a serine protease and a second different protease, e.g. an MMP. In some aspects, a CM can serve as a substrate for more than one serine protease, e.g., a matriptase and/or uPA. In some aspects, a CM can serve as a substrate for more than one MMP, e.g., MMP9 and MMP14.

In some aspects of the present disclosure, CM1, CM2, CM3, and CM4 each independently comprise a substrate for a protease selected from Table 1.

TABLE 1
Exemplary Proteases

| | | |
|---|---|---|
| ADAMS, ADAMTS, e.g. | Cysteine proteinases, e.g., | Serine proteases, e.g., |
| ADAM8 | Cruzipain | activated protein C |
| ADAM9 | Legumain | Cathepsin A |
| ADAM10 | Otubain-2 | Cathepsin G |
| ADAM12 | KLKs, e.g., | Chymase |
| ADAM15 | KLK4 | coagulation factor proteases |
| ADAM17/TACE | KLK5 | (e.g., FVIIa, FIXa, FXa, FXIa, |
| ADAMDEC1 | KLK6 | FXIIa) |
| ADAMTS1 | KLK7 | Elastase |
| ADAMTS4 | KLK8 | Granzyme B |
| ADAMTS5 | KLK10 | Guanidinobenzoatase |
| Aspartate proteases, e.g., | KLK11 | HtrA1 |
| BACE | KLK13 | Human Neutrophil Elastase |
| Renim | KLK14 | Lactoferrin |
| Aspartic cathepsins, e.g., | Metallo proteinases, e.g., | Marapsin |
| Cathepsin D | Meprin | NS3/4A |
| Cathepsin E | Neprilysin | PACE4 |
| Caspases, e.g., | PSMA | Plasmin |
| Caspase 1 | BMP-1 | PSA |
| Caspase 2 | MMPs, e.g., | tPA |
| Caspase 3 | MMP1 | Thrombin |
| Caspase 4 | MMP2 | Tryptase |
| Caspase 5 | MMP3 | uPA |
| Caspase 6 | MMP7 | Type II Transmembrane |
| Caspase 7 | MMP8 | Serine Proteases (TTSPs), e.g., |
| Caspase 8 | MMP9 | DESC1 |
| Caspase 9 | MMP10 | DPP-4 |
| Caspase 10 | MMP11 | FAP |
| Caspase 14 | MMP12 | Hepsin |
| Cysteine cathepsins, e.g., | MMP13 | Matriptase-2 |
| Cathepsin B | MMP14 | MT-SP1/Matriptase |
| Cathepsin C | MMP15 | TMPRSS2 |
| Cathepsin K | MMP16 | TMPRSS3 |
| Cathepsin L | MMP17 | TMPRSS4 |
| Cathepsin S | MMP20 | |
| Cathepsin V/L2 | MMP23 | |
| Cathepsin X/Z/P | MMP24 | |
| | MMP26 | |
| | MMP27 | |

In some aspects, the CM1, CM2, CM3 and/or CM4 may comprise two or more cleavage sites. In some aspects of activatable anti-EGFR, anti-CD3 heteromultimeric bispecific polypeptide complex described herein, the CM1, CM2, CM3 and/or CM4 each independently include about three amino acids to about 15 amino acids. In some aspects, the first protease and the second protease are the same protease. In some aspects, CM1 and CM2 are different substrates for the same protease. In some aspects, CM3 and CM4 are different substrates for different proteases. In some aspects, CM1 and CM2 comprise the same amino acid sequence. In some aspects, CM3 and CM4 comprise the same amino acid sequence. In some aspects, the first protease and the second protease (i.e., where CM1 comprises a substrate for a first protease, and CM2 comprises a substrate for a second protease) are different, and CM1 and CM2 comprise different amino acid sequences. In some aspects, the third protease and the fourth protease (i.e., wherein CM3 comprises a substrate for a third protease, and CM4 comprises a substrate for a fourth protease) are different, and CM3 and CM4 comprise different amino acid sequences. In some aspects, CM1 comprises the amino acid sequence SEQ ID NO:5. In some aspects, CM3 comprises the amino acid sequence SEQ ID NO:5. In some aspects, CM2 comprises the amino acid sequence SEQ ID NO:19. In some aspects, CM4 comprises the amino acid sequence SEQ ID NO:19.

Exemplary CMs that are suitable for use in the activatable anti-EGFR, anti-CD3 heteromultimeric bispecific polypeptide described herein are known in the art. Exemplary CMs include but are not limited to those described in, for example, PCT Publication Nos.: WO 2009/025846, WO 2010/081173, WO 2015/013671, WO 2015/048329, WO 2015/116933, WO 2016/014974, and WO 2016/118629, each of which is incorporated herein by reference in its entirety.

In some aspects CM1, CM2, CM3, and/or CM4 include a substrate for a protease selected from the group consisting of a serine protease and a matrix metallopeptidase (MMP). In some aspects, CM1, CM2, CM3, and/or CM4 include a substrate for a serine, such as, for example, matriptase or urokinase plasminogen activator (uPA). In some aspects, CM1, CM2, CM3, and/or CM4 include a substrate for an MMP, such as, for example MMP9, MMP14, and the like.

In some aspects, at least one of CM1, CM2, CM3, and CM4 comprises an amino acid sequence set forth in Table 2 below.

TABLE 2
Cleavable Moieties

| CM SEQ ID NO. | CM SEQUENCE |
|---|---|
| 5 | LSGRSDDH |
| 19 | ISSGLLSGRSDQH |
| 57 | LSGRSDNH |
| 58 | TSTSGRSANPRG |
| 59 | VHMPLGFLGP |
| 60 | AVGLLAPP |
| 61 | QNQALRMA |
| 62 | ISSGLLSS |
| 63 | ISSGLLSGRSDNH |
| 65 | LSGRSDIH |
| 66 | LSGRSDQH |
| 67 | LSGRSDTH |
| 68 | LSGRSDYH |
| 69 | LSGRSDNP |
| 70 | LSGRSANP |
| 71 | LSGRSANI |
| 72 | LSGRSDNI |
| 73 | ISSGLLSGRSANPRG |
| 74 | AVGLLAPPTSGRSANPRG |
| 75 | AVGLLAPPSGRSANPRG |
| 76 | ISSGLLSGRSDDH |
| 77 | ISSGLLSGRSDIH |
| 78 | ISSGLLSGRSDTH |
| 79 | ISSGLLSGRSDYH |
| 80 | ISSGLLSGRSDNP |
| 81 | ISSGLLSGRSANP |
| 82 | ISSGLLSGRSANI |
| 83 | AVGLLAPPGGLSGRSDDH |
| 84 | AVGLLAPPGGLSGRSDIH |
| 85 | AVGLLAPPGGLSGRSDQH |
| 86 | AVGLLAPPGGLSGRSDTH |
| 87 | AVGLLAPPGGLSGRSDYH |
| 88 | AVGLLAPPGGLSGRSDNP |
| 89 | AVGLLAPPGGLSGRSANP |
| 90 | AVGLLAPPGGLSGRSANI |
| 91 | ISSGLLSGRSDNI |
| 92 | AVGLLAPPGGLSGRSDNI |
| 93 | ISSGLLSGRSGNH |
| 129 | ALAHGLF |
| 130 | APRSALAHGLF |
| 131 | ISSGLLSGRSNI |
| 132 | LSGRSNI |

In some aspects of the activatable anti-EGFR, anti-CD3 heteromultimeric bispecific polypeptides (HBPCs) of the present disclosure, the first, second, third, and/or fourth polypeptides comprise one or more linkers (e.g., L1, L2, L3, L4, etc.). In some aspects, a linker is present between an MM and a CM. In some aspects, MM1 is joined to CM1 via a linker. In some aspects, MM2 is joined to CM2 via a linker. In some aspects, MM3 is joined to CM3 via a linker. In some aspects, MM4 is joined to CM4 via a linker. In some aspects, the activatable anti-EGFR, anti-CD3 heteromultimeric bispecific polypeptide comprises a linker between a CM and an antigen-binding variable domain (e.g., VH1, VL1, VH2, VL2, VH3, VL3, VH4, and/or VL4). Linkers suitable for use in the activatable anti-EGFR, anti-CD3 heteromultimeric bispecific polypeptides (HBPCs) described herein are generally ones that provide flexibility of the activatable anti-EGFR, anti-CD3 heteromultimeric bispecific polypeptides (HBPCs) to facilitate the inhibition of the binding of the activatable polypeptide to the target. Such linkers are generally referred to as flexible linkers. In some aspects, the activatable HBPC comprises two or more linkers having the same amino acid sequence. In some aspects, the amino acid sequences of each linker is different.

Suitable linkers can be readily selected and can be of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, 7, 8, 9, 10, 11, 12, 13, 14, 15, 16, 17, 18, 19, or 20 amino acids in length.

Exemplary flexible linkers include glycine polymers (G) n, glycine-serine polymers (including, for example, (GS) n, (GSGGS) n and (GGGS) n (SEQ ID NO:95), wherein n is an integer of at least one, and in some aspects, wherein n is an integer from 1 to 10, (SEQ ID NO:94), where n is an integer of at least one, and in some aspects, wherein n is an integer from 1 to 10, glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine, and is much less restricted than residues with longer side chains (see Scheraga, Rev. Computational Chem. 11173-142 (1992)). The ordinarily skilled artisan will recognize that design of an activatable polypeptides can include linkers that are all or partially flexible, such that the linker can include a flexible linker as well as one or more portions that confer less flexible structure to provide for a desired structure.

In some aspects, the activatable anti-EGFR, anti-CD3 HBPCs can comprise one or more linker sequences that can be disposed in the first, second, third, and/or fourth polypeptides. For example in the first polypeptide, a linker can be present between the MM1 and the CM1, between the VH1 and VL1, between a VL1 and a VH2, between a VH2 and a CH1 domain, between a CH1 domain and a hinge region if both are present, and/or between a hinge region if present and the first Fc domain. In the second polypeptide, which is described elsewhere herein, a linker can be present, for example, between the MM2 and the CM2, between the CM2 and the VL2, and/or between the VL2 and a CL if present. In the third polypeptide, a linker can be present between the MM3 and the CM3, between the CM3 and VL3, between the VL3 and the VH4, between the VH4 and a CH1 domain (if present), between a CH1 domain and a hinge region if both are present, and/or between a hinge region if present and the second Fc domain. In the fourth polypeptide, which is described elsewhere herein, a linker can be present, for example, between the MM4 and the CM4, between the CM4 and the VL4, and/or between the VL4 and a CL if present.

In some aspects of the activatable anti-EGFR, anti-CD3 heteromultimeric bispecific polypeptide complexes (HBPCs) described herein, MM1 is linked to CM1 via linker L1. In some aspects, MM2 is linked to CM2 via linker L2. In some aspects, MM3 is linked to CM3 via linker L3. In some aspects, MM4 is linked to CM4 via linker L4. In some aspects, the amino acid sequence of L1, L2, L3 and/or L4 are the same. In some aspects, the amino acid sequence of L1, L2, L3 and/or L4 are different. In some aspects, the linker is selected from the group consisting of: (i) a glycine-serine-based linker selected from the group consisting of (GS) n, wherein n is an integer of at least 1, (GGS) n, wherein n is an integer of at least 1, and in some aspects, wherein n is an integer from 1 to 10, (GGGS) n (SEQ ID NO:94), wherein n is an integer of at least 1, and in some aspects, wherein n is an integer from 1 to 10, (GS) n, wherein n is an integer of at least 1, and in some aspects, wherein n is an integer from 1 to 10, (GGS) n, wherein n is an integer of at least 1, and in some aspects, wherein n is an integer from 1 to 10, (GGGGS) n (SEQ ID NO: 124), wherein n is an integer of at least 1, and in some aspects, wherein n is an integer from 1 to 10, (GGGS) n (SEQ ID NO:94), wherein n is an integer of at least 1, and in some aspects, wherein n is an integer from 1 to 10, (GSGGS) n (SEQ ID NO:95), wherein n is an integer of at least 1, and in some aspects, wherein n is an integer from 1 to 10, GGSG (SEQ ID NO:96), GGSGG (SEQ ID NO:97), GSGSG (SEQ ID NO:98), GSGGG (SEQ ID NO:99), GGGSG (SEQ ID NO:100), and GSSSG (SEQ ID NO:101), GGGGGGGGSGGGGSGS (SEQ ID NO:102), GGGGSGS (SEQ ID NO:103), GGGGSGGGGSGGGGS (SEQ ID NO:104), GGGGSGGGGSGGGGSGGGGS (SEQ ID NO: 105), GGGGS (SEQ ID NO:106), GGGGSGGGGS (SEQ ID NO:107), GGGSGGGS (SEQ ID NO:108), GGGSGGGSGGGS (SEQ ID NO:109), GSSGGSGGSGG (SEQ ID NO: 110), GGGSGGGGSGGGGSGGGGSGGGGS (SEQ ID NO:111), GGGSSGGS (SEQ ID NO:123), GSSGGSGGSG (SEQ ID NO:125) and GS; and (ii) a linker comprising glycine and serine, and at least one of lysine, threonine, or proline selected from the group consisting of GSTSGSGKPGSSEGST (SEQ ID NO:112), SKYGPPCPPCPAPEFLG (SEQ ID NO:113), GGSLDPKGGGGS (SEQ ID NO:114), PKSCDKTHTCPPCPAPELLG (SEQ ID NO:115), GKSSGSGSESKS (SEQ ID NO: 116), GSTSGSGKSSEGKG (SEQ ID NO:117), GSTSGSGKSSEGSGSTKG (SEQ ID NO: 118), and GSTSGSGKPGSGEGSTKG (SEQ ID NO:119).

In some aspects of the present disclosure, an activatable anti-EGFR, anti-CD3 heteromultimeric bispecific polypeptide can comprise components in addition to those described above. Such components can include a spacer. The term “spacer” refers herein to an amino acid residue or a peptide incorporated at a free terminus of the first, second, and/or third polypeptide. Spacers that are suitable for use in the practice of the present disclosure include any single amino acid residue or any peptide. Suitable spacers include any of those described in, for example, WO 2016/014974, WO 2019/075405, and WO 2019/213444, each of which is incorporated herein by reference in their entireties.

In some aspects, a spacer can comprise from about 1 amino acid to about 10 amino acids (e.g., about 1, 2, 3, 4, 5, 6, 7, 8, or 9 amino acids) or any number therebetween. In some aspects of an activatable anti-EGFR, anti-CD3 heteromultimeric bispecific polypeptide described herein, the spacer is N-terminally positioned relative to the MM1, MM2, MM3, and/or MM4. In some aspects, the spacer has a sequence of QGQSGS (SEQ ID NO:3). In some aspects, the spacer has a sequence of QGQSGQG (SEQ ID NO:17). In certain aspects, a linker is disposed between the spacer and a masking moiety (i.e., MM1, MM2, MM3, and/or MM4). Suitable linkers include any linkers known in the art, and any of the linkers described hereinabove.

In some aspects, the Fc domains employed as Fc1 and/or Fc2 are native Fc domains (e.g., a human IgG1 Fc domain or a human IgG4 Fc domain). In some aspects of the present disclosure, the Fc domains employed as Fc1 and/or Fc2 are mutated forms of a native Fc amino acid sequence. The mutations may confer a desired beneficial property to the activatable anti-EGFR, anti-CD3 heteromultimeric bispecific polypeptide (and commensurately, the activated HBPC). For example, certain mutations in the FcRn binding site are known to modulate effector function (see, e.g., Petkova et al., Intl. Immunol. 18:1759-1769, 2006; Deng et al., MAbs 4:101-109, 2012; and Olafson et al., Methods Mol. Biol. 907:537-556, 2012.) The inclusion of any known mutations in an Fc domain that can modulate effector function are suitable. For example, a N297A or N97G mutation in the Fc amino acid sequence may be employed to reduce IgG effector functions (e.g., ADCC and CDC) which may reduce target independent toxicities (see, e.g., Lund et al., Mol. Immunol. 29:35-39, 1992). The Fc domains suitable for use in the invention include any Fc domain known in the art, including but not limited to any known heterodimeric Fc (e.g., knob-in-holes).

In some aspects, the first and second Fc domains (Fc1 and Fc2, respectively) of the activatable anti-EGFR, anti-CD3 HBPC described herein are IgG1 Fc domains or IgG4 Fc domains (e.g., a human IgG1 Fc domain or a human IgG4 Fc domain) or variants thereof. In some aspects, Fc1 and/or Fc2 are modified variants of a native (e.g., human) IgG4 Fc domain.

In some aspects, the third polypeptide further comprises a monomeric Fc domain (Fc2) that binds to Fc1. In some aspects, Fc2 comprises an amino acid sequence that is at least 90%, at least 91%, at least 92%, at least 93%, at least 94%, at least 95%, at least 96%, at least 97%, at least 98%, or at least 99% identical to SEQ ID NO:28. In some aspects, the Fc2 comprises SEQ ID NO:28.

In some aspects, the first polypeptide Fc1 and third polypeptide Fc2 hinge regions comprise the same sequence. In some aspects, the first polypeptide Fc1 and third polypeptide Fc2 hinge regions comprise different sequences. In some aspects, a hinge region comprises the amino acid sequence of EPKSCDKTHTCPPC (SEQ ID NO:10). In some aspects, a hinge region comprises the amino acid sequence of DKTHTCPPC (SEQ ID NO: 11).

As provided elsewhere herein, the format or structure of an activatable anti-EGFR, anti-CD3 heteromultimeric bispecific polypeptide complex disclosed herein can include any number of optional additional components, including linkers and spacers. By way of example only, the structures set forth below are among the contemplated aspects. However, the aspects shown below are not meant to limit the disclosure in any way.

In some aspects, the activatable anti-EGFR, anti-CD3 heteromultimeric bispecific polypeptide comprises a first polypeptide having a structure (I).

(S1)-MM1-(L¹)-CM1-L²-VH1-L³-VL1-(L⁴)-VH2-(L⁵)--(CH1¹)-(L⁶)-(Hinge¹)--(L⁷)-Fc1,

wherein:
(S1) is an optional spacer that is present or absent;
(L¹), (L⁴), (L⁵), (L⁶), and (L⁷) are each independently an optional linker that is present or absent,
   L² and L³ are linkers,
(CH1¹) is an optional CH1 domain that is present or absent,
(Hinge¹) is an optional hinge region that is present or absent,
MM1 is an anti-CD3 MM,
VH1 is an anti-CD3 heavy chain variable domain,
VL1 is an anti-CD3 heavy chain variable domain, and
VH2 is an anti-EGFR heavy chain variable domain.

In some aspects, the second polypeptide comprises the structure (II):

(S2)-(L⁸)-MM2-(L⁹)-CM2-(L¹⁰)-VL2-(CL)

wherein
(S2) is an optional spacer that is present or absent,
(L⁸), (L⁹), and (L¹⁰) are each independently an optional linker that is present or absent,
(CL) is an optional light chain constant domain that is present or absent,
MM2 is an anti-EGFR masking moiety, and
VL2 is an anti-EGFR light chain variable domain;
In some aspects, the third polypeptide comprises the structure (III):

(S1)-MM3-(L¹)-CM3-L²-VH3-L³-VL3-(L⁴)-VH4-(L⁵)--(CH1¹)-(L⁶)-(Hinge¹)-(L⁷)-Fc2,

wherein
(S1) is an optional spacer that is present or absent;
(L¹), (L⁴), (L⁵), (L⁶), and (L⁷) are each independently an optional linker that is present or absent,
L² and L³ are each a linker,
(CH1¹) is an optional CH1 domain that is present or absent,
(Hinge¹) is an optional hinge region that is present or absent,
MM3 is an anti-CD3 MM,
VH3 is an anti-CD3 heavy chain variable domain,
VL3 is an anti-CD3 heavy chain variable domain, and
VH4 is an anti-EGFR HVD; and

In some aspects, the fourth polypeptide comprises the structure (IV):

(S2)-(L⁸)-MM4-(L⁹)-CM4-(L¹⁰)-VL4-(CL)

wherein
(S2) is an optional spacer that is present or absent,
(L⁸), (L⁹), and (L¹⁰) are each independently an optional linker that is present or absent,
(CL) is an optional light chain constant domain that is present or absent,
MM4 is an anti-EGFR masking moiety, and
VL4 is an anti-EGFR light chain variable domain.

Linkers, spacers, MMs, CMs, Fc domains, CH1 domains, hinge regions, and CLs that are suitable for use in polypeptides (I), (II), (III), and (IV) include any that are known in the art or that are described herein.

In some aspects, the present disclosure provides the activatable anti-EGFR, anti-CD3 heteromultimeric bispecific polypeptide of Complex-66, comprising first, second, third, and fourth polypeptides, as provided below.

As shown in the sequences below, in the first and third polypeptides, the spacer is underscored, MM/MM3 is bolded, linkers are italicized, CM1/CM3 is italicized and underscored, the heavy chain variable domain (VH1 and VH3) is bracketed, the light chain variable domain (VL1 and VL3) is bracketed and italicized, the second and fourth heavy chain variable domain (VH2 and VH4) is bracketed and underscored, and the CH1-hinge region-first Fc domain is bracketed, italicized, and underscored.

First and Third Polypeptides
(SEQ ID NO: 2)
QGQSGSVSTTCWWDPPCTPNTGSSGGSGGSGGLSGRSDDHGGGS[EVQLVESGGGLV
QPGGSLKLSCAASGFTFNKYAMNWVRQAPGKGLEWVARIRSKYNNYATYYADSVK
DRFTISRDDSKNTAYLQMNNLKTEDTAVYYCVRHGNFGNSYISYWAYWGQGTLVT
VSS]GGGGSGGGGSGGGGS[QTVVTQEPSLTVSPGGTVTLTCGSSTGAVTSGNYPNW
VQQKPGQAPRGLIGGTKFLAPGTPARFSGSLLGGKAALTLSGVQPEDEAEYYCVLW
YSNRWVFGGGTKLTVL]GGGGS[QVQLKQSGPGLVQPSQSLSITCTVSGFSLTNYGVH
WVRQSPGKGLEWLGVIWSGGNTDYNTPFTSRLSINKDNSKSQVFFKMNSLQSQDTAI
YYCARALTYYDYEFAYWGQGTLVTVSA][ASTKGPSVFPLAPSSKSTSGGTAALGCLVK
DYFPEPVTVSWNSGALTSGVHTEPAVLQSSGLYSLSSVVTVPSSSLGTQTYICNVNHKPSNT
KVDKKVEPKSCDKTHTCPPCPAPELLGGPSVFLFPPKPKDTLMISRTPEVTCVVVDVSHE
DPEVKFNWYVDGVEVHNAKTKPCEEQYGSTYRCVSVLTVLHQDWLNGKEYKCKVSNKAL
PAPIEKTISKAKGQPREPQVYTLPPSRKEMTKNQVSLTCLVKGFYPSDIAVEWESNGQPEN
NYKTTPPVLKSDGSFFLYSKLTVDKSRWQQGNVFSCSVMHEALHNHYTQKSLSLSPG

As shown below, in the second and fourth polypeptides, the spacer is underscored, MM2/MM4 is bolded, the linkers are italicized, CM2/CM is italicized and underscored, the light chain variable region (VL2 and VL4) is bracketed, and the light chain constant region is italicized and underscored.

Second and Fourth Polypeptides
(SEQ ID NO: 16]
QGQSGQGLSCEGWAMNREQCRAGGGSSGGSISSGLLSGRSDQHGGGS[QILLTQSPVI
LSVSPGERVSFSCRASQSIGTNIHWYQQRTNGSPRLLIKYASESISGIPSRFSGSGSGTD
FTLSINSVESEDIADYYCQQNNNWPTTFGAGTKLELK]RTVAAPSVFIFPPSDEQLKSGT
ASVVCLLNNFYPREAKVQWKVDNALQSGNSQESVTEQDSKDSTYSLSSTLTLSKADYEKHK
VYACEVTHQGLSSPVTKSFNRGEC

### Kits

Provided herein are kits comprising one or more activatable anti-EGFR, anti-CD3 HBPC described herein, wherein the kits are useful for diagnostic or treatment. In certain aspects, provided herein is a pack or kit comprising one or more containers filled with one or more of the ingredients of the compositions described herein, such as one or more an activatable anti-EGFR, anti-CD3 heteromultimeric bispecific polypeptides (HBPCs) provided herein, and optionally, an instruction for use. In some aspects, the kits contain a composition described herein and any diagnostic, prophylactic or therapeutic agent, such as those described herein.

### Therapeutic Uses and Methods

In some aspects, presented herein are methods treating diseases, e.g., cancers, comprising administering to a subject in need thereof an activatable anti-EGFR, anti-CD3 HBPC described herein, or a pharmaceutical composition thereof as described herein. In some aspects, presented herein are methods of inhibiting tumor growth in a subject in need thereof comprising administering to a subject in need thereof an activatable anti-EGFR, anti-CD3 heteromultimeric bispecific polypeptide described herein, or a pharmaceutical composition thereof as described herein. In some aspects, the present disclosure relates to an activatable anti-EGFR, anti-CD3 heteromultimeric bispecific polypeptide or pharmaceutical composition provided herein for use as a medicament. Usually, the subject is a human, but non-human mammals including transgenic mammals can also be treated.

The amount of HBPC or composition which will be effective in the treatment of a condition will depend on the nature of the disease. The precise dose to be employed in a composition will also depend on the route of administration, and the seriousness of the disease.

Non-limiting examples of disease include: cancers, rheumatoid arthritis, Crohn's disease, SLE, cardiovascular damage, ischemia, etc. For example, indications can include leukemias, including T-cell acute lymphoblastic leukemia (T-ALL), lymphoblastic diseases including multiple myeloma, and solid tumors, including lung, colorectal, prostate, pancreatic and breast, including triple negative breast cancer. For example, indications can include bone disease or metastasis in cancer, regardless of primary tumor origin; breast cancer, including by way of non-limiting example, ER/PR+ breast cancer, Her2+ breast cancer, triple-negative breast cancer; colorectal cancer; endometrial cancer; gastric cancer; glioblastoma; head and neck cancer, such as head and neck squamous cell cancer; esophageal cancer; lung cancer, such as by way of non-limiting example, non-small cell lung cancer; multiple myeloma ovarian cancer; pancreatic cancer; prostate cancer; sarcoma, such as osteosarcoma; renal cancer, such as by way of non-limiting example, renal cell carcinoma; and/or skin cancer, such as by way of non-limiting example, squamous cell cancer, basal cell carcinoma, or melanoma.

### Polynucleotides

In some aspects, provided herein are polynucleotides comprising a nucleotide sequence encoding the first, second, third, and or fourth polypeptide of an activatable anti-EGFR, anti-CD3 heteromultimeric bispecific polypeptide complex of the present invention (correspondingly referred to herein as the “first polynucleotide,” the “second polynucleotide,” the “third polynucleotide,” and the “fourth polynucleotide,” respectively). Suitable polynucleotides include any that encode any of the first, second, third, and/or fourth polypeptides described herein, or portion thereof. An illustrative set of polynucleotide sequences encoding a first, second, third, and fourth polypeptide is provided herein below.

Polynucleotides of the present invention may be sequence optimized for optimal production from the host organism selected for expression, e.g., by codon/RNA optimization, replacement with heterologous signal sequences, and elimination of mRNA instability elements. Methods to generate optimized nucleic acids encoding an activatable anti-EGFR, anti-CD3 heteromultimeric bispecific polypeptide complex (e.g., VH domain, or VL domain) for recombinant expression by introducing codon changes (e.g., a codon change that encodes the same amino acid due to the degeneracy of the genetic code) and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly.

Nucleic Acids
First and Third Polypeptides
(SEQ ID NO: 1)
CAAGGACAATCTGGCTCTGTGTCCACCACCTGTTGGTGGGACCCTCCATGCACACCT
AATACCGGCAGCTCTGGTGGCTCTGGCGGAAGCGGAGGACTGTCTGGCAGATCCGA
TGATCACGGCGGAGGATCTGAGGTGCAGCTGGTTGAATCTGGTGGCGGACTGGTTCA
GCCTGGCGGATCTCTGAAACTGAGCTGTGCCGCCAGCGGCTTCACCTTCAACAAATA
CGCCATGAACTGGGTCCGACAGGCCCCTGGCAAAGGCCTTGAATGGGTCGCCAGAA
TCAGAAGCAAGTACAACAACTATGCCACCTACTACGCCGACAGCGTGAAGGACAGA
TTCACCATCAGCCGGGACGACAGCAAGAACACCGCCTACCTGCAGATGAACAACCT
GAAAACCGAGGACACCGCCGTGTACTACTGTGTGCGGCACGGCAACTTCGGCAACA
GCTACATCAGCTACTGGGCCTATTGGGGCCAGGGCACACTGGTCACAGTTTCTAGTG
GCGGAGGCGGATCTGGCGGCGGTGGAAGTGGCGGCGGAGGTTCTCAAACAGTGGTC
ACCCAAGAGCCTAGCCTGACCGTTTCTCCTGGCGGAACCGTGACACTGACATGCGGA
TCTTCTACAGGCGCCGTGACCAGCGGCAACTACCCTAATTGGGTGCAGCAGAAGCCA
GGCCAGGCTCCTAGAGGACTGATCGGCGGCACAAAGTTTCTGGCTCCCGGAACACC
AGCCAGATTCAGCGGTTCTCTGCTCGGAGGAAAGGCCGCTCTGACACTTTCTGGCGT
GCAGCCTGAGGATGAGGCCGAGTACTATTGCGTGCTGTGGTACAGCAACAGATGGG
TGTTCGGCGGAGGCACCAAGCTGACAGTTCTTGGAGGTGGCGGTAGCCAGGTCCAG
CTGAAACAATCTGGACCCGGACTCGTGCAGCCAAGCCAGAGCCTGTCTATCACCTGT
ACCGTGTCCGGCTTCAGCCTGACCAATTACGGCGTGCACTGGGTTCGACAATCTCCC
GGCAAGGGACTCGAATGGCTGGGAGTGATTTGGAGCGGCGGCAACACCGACTACAA
CACCCCATTCACCAGCAGACTGAGCATCAACAAGGACAACAGCAAGTCCCAGGTGT
TCTTCAAGATGAACTCCCTGCAGAGCCAGGATACCGCCATCTATTACTGCGCTCGGG
CCCTGACCTACTATGACTACGAGTTTGCCTACTGGGGACAGGGAACCCTCGTGACAG
TGTCTGCTGCTAGCACAAAGGGCCCTAGCGTTTTCCCACTGGCTCCCAGCAGCAAGT
CTACATCCGGTGGAACAGCCGCTCTGGGCTGCCTGGTCAAGGATTACTTTCCCGAGC
CAGTGACCGTGTCCTGGAATAGCGGAGCACTGACATCTGGCGTGCACACATTTCCAG
CCGTGCTGCAGTCTAGCGGCCTGTACTCTCTGTCCAGCGTTGTGACAGTGCCCAGCA
GCTCTCTGGGCACCCAGACCTACATCTGCAATGTGAACCACAAGCCTAGCAACACCA
AGGTGGACAAGAAGGTGGAACCCAAGAGCTGCGATAAGACACACACCTGTCCTCCA
TGTCCTGCTCCAGAGCTGCTCGGAGGCCCTTCCGTGTTTCTGTTCCCTCCAAAGCCTA
AGGACACCCTGATGATCAGCAGAACCCCTGAAGTGACCTGCGTGGTGGTGGATGTG
TCCCACGAGGATCCCGAAGTGAAGTTCAATTGGTACGTCGACGGCGTGGAAGTGCA
CAATGCCAAGACCAAGCCTTGCGAGGAACAGTACGGCAGCACCTACAGATGCGTGT
CCGTGCTGACAGTGCTGCACCAGGATTGGCTGAACGGCAAAGAGTACAAGTGCAAG
GTGTCCAACAAGGCCCTGCCTGCTCCTATCGAGAAAACCATCAGCAAGGCCAAGGG
CCAGCCTAGAGAACCCCAGGTGTACACACTGCCTCCAAGCCGGAAAGAGATGACCA
AGAATCAGGTGTCCCTGACCTGCCTGGTCAAGGGCTTCTACCCTTCCGATATCGCCG
TGGAATGGGAGAGCAATGGACAGCCCGAGAACAACTACAAGACAACCCCTCCTGTG
CTGAAGTCCGACGGCTCATTCTTCCTGTACAGCAAGCTGACCGTGGACAAGAGCAGA
TGGCAGCAGGGCAACGTGTTCAGCTGCAGCGTGATGCACGAGGCCCTGCACAACCA
CTACACCCAGAAGTCCCTGTCTCTGAGCCCCGGCAAA

The C-terminal lysine in the encoded polypeptide is not present in the purified protein. In a variation of this illustrative polynucleotide, the codon encoding the C-terminal lysine may be absent (i.e., SEQ ID NO:126).

Second and Fourth Polypeptides
(SEQ ID NO: 15)
CAAGGACAATCTGGACAGGGCCTGAGCTGTGAAGGCTGGGCCATGAATAGAGAGCA
GTGCAGAGCTGGCGGCGGATCTTCTGGCGGCTCTATCTCTTCTGGACTGCTGAGCGG
CAGAAGCGATCAACACGGCGGAGGCTCTCAGATCCTGCTGACACAGAGCCCCGTGA
TCCTGTCTGTGTCTCCTGGCGAGAGAGTGTCCTTCAGCTGTAGAGCCAGCCAGTCCA
TCGGCACCAACATCCACTGGTATCAGCAGCGGACCAACGGCAGCCCCAGACTGCTG
ATTAAGTACGCCAGCGAGAGCATCAGCGGCATCCCCAGCAGATTTTCTGGCAGCGG
CTCTGGCACCGACTTCACCCTGAGCATCAACAGCGTGGAAAGCGAGGATATCGCCG
ACTACTACTGCCAGCAGAACAACAACTGGCCCACCACCTTTGGAGCCGGCACCAAG
CTGGAACTGAAGAGAACAGTGGCCGCTCCTAGCGTGTTCATCTTCCCACCTTCCGAC
GAGCAGCTGAAAAGCGGCACAGCCTCTGTCGTGTGCCTGCTGAACAACTTCTACCCC
AGAGAAGCCAAGGTGCAGTGGAAGGTGGACAACGCCCTGCAGAGCGGCAATAGCC
AAGAGTCTGTGACCGAGCAGGACAGCAAGGACTCCACCTACAGCCTGAGCAGCACC
CTGACACTGAGCAAGGCCGACTACGAGAAGCACAAAGTGTACGCCTGCGAAGTGAC
CCACCAGGGCCTTTCTAGCCCTGTGACCAAGAGCTTCAACCGGGGCGAGTGT

A polynucleotide encoding a polypeptide or antigen-binding fragment thereof described herein or a domain thereof can be generated from nucleic acid from a suitable source (e.g., a hybridoma) using methods well known in the art (e.g., PCR and other molecular cloning methods), synthesized using techniques that are well known in the art, and the like. Polynucleotides encoding the first, second, and third polypeptides can be cloned into one or more vectors for expression in host cells and for further cloning, for example, to generate chimeric and humanized antibodies or antigen-binding fragments thereof.

Polynucleotides provided herein can be an RNA or a DNA. DNA includes cDNA, genomic DNA, and synthetic DNA, and DNA can be double-stranded or single-stranded. If single stranded, DNA can be the coding strand or non-coding (anti-sense) strand. In some aspects, the polynucleotide is a cDNA or a DNA lacking one more endogenous introns. In some aspects, a polynucleotide is a non-naturally occurring polynucleotide. In some aspects, a polynucleotide is recombinantly produced. In some aspects, the polynucleotides are isolated. In some aspects, the polynucleotides are substantially pure. In some aspects, a polynucleotide is purified from natural components.

In some aspects, the polynucleotides described herein encode an activatable anti-EGFR, anti-CD3 heteromultimeric bispecific polypeptide complexes (HBPCs) that comprise the heavy chains (first and third polypeptides) and light chains (second and fourth polypeptides), including the variable domains and CDRs provided herein.

### Vectors, Host Cells, and Methods of Production

Provided herein are one or more vectors comprising polynucleotides encoding the first, second, third, and fourth polypeptides of the present invention (corresponding to a first polynucleotide, a second polynucleotide, a third polynucleotide, and a fourth polypeptide, respectively). In some embodiments, such vectors may be used to recombinantly produce the polypeptides of the activatable HBPC from a host cell, as described in more detail hereinbelow. In some aspects, the vector comprises the first, the second, the third, and/or the fourth polynucleotide operably linked to one or more promoter sequences. In certain aspects, the present invention provides a plurality of vectors that collective comprise the polynucleotides encoding the first, second, third, and fourth polypeptides (i.e., the first, second, third, and fourth polynucleotides), where the plurality comprises at least one vector that comprises no more than two, or no more than one of the first, the second, the third, and the fourth polynucleotides. In these embodiments, the first, the second, the third, and the fourth polynucleotide sequences in the plurality of vectors are usually operably linked to one or more promoter sequences.

Also provided herein are recombinant host cells comprising any of the above-described polynucleotides and/or vectors for recombinantly expressing the polynucleotides encoding the polypeptides of the activatable anti-EGFR, anti-CD3 heteromultimeric bispecific polypeptide complex of the present invention. A variety of host-expression vector systems can be utilized to express the polypeptides described herein (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express described polypeptide described herein in situ. Exemplary host cells that are suitable for use as a recombinant expression host for the above-described polynucleotides include mammalian cell systems (e.g., COS (e.g., COS1 or COS), CHO, BHK, MDCK, HEK 293, NSO, PER.C6, VERO, CRL7O3O, HsS78Bst, HeLa, and NIH 3T3, HEK-293T, HepG2, SP210, R1.1, B-W, L-M, BSC1, BSC40, YB/20, BMT10 cells, and the like). Vectors employed in the construction of a recombinant mammalian host cell may comprise a promoter derived from the genome of a mammalian cell (e.g., metallothionein promoter) or from a mammalian virus (e.g., the adenovirus late promoter; the vaccinia virus 7.5K promoter). In some aspects, the recombinant host cell is a CHO cell or a NS0 cell.

In some aspects, recombinant expression of a polypeptide described herein, e.g., a first, second, third, and/or fourth polypeptide, involves construction of an expression vector containing a polynucleotide that encodes the activatable anti-EGFR, anti-CD3 HBPC. Vector(s) comprising polynucleotides encoding the activatable HBPC can be readily generated by recombinant DNA technology using techniques well known in the art. Methods which are well known to those skilled in the art can be used to construct expression vectors containing one or more polynucleotides encoding the polypeptides described herein, e.g., a first, second, third, and/or fourth polypeptide, as well as appropriate transcriptional and translational control signals. These methods include, for example, in vitro recombinant DNA techniques, synthetic techniques, and in vivo genetic recombination. Also provided are replicable vectors comprising a nucleotide sequence operably linked to a promoter. Such vectors can, for example, include the nucleotide sequence encoding the constant region of a polypeptide described herein, e.g., a first, second, third, and/or fourth polypeptide (see, e.g., International Publication Nos. WO 86/05807 and WO 89/01036; and U.S. Pat. No. 5,122,464), and variable domains of the polypeptide can be cloned into such a vector for expression of the entire VH, the entire VL, or both the entire VH and VL.

An expression vector can be transferred to a cell (e.g., host cell) by conventional techniques and the resulting cells can then be cultured by conventional techniques to produce the activatable HBPC described herein (e.g., the CDRs, the VH1, VH2, VH3, VH4, and the VL1, VL2, VL3, and VL4 of an activatable anti-EGFR, anti-CD3 HBPC provided herein). Thus, provided herein are host cells containing a polynucleotide encoding the HBPC described herein, operably linked to a promoter for expression of such sequences in the host cell. In some aspects, a host cell contains a vector comprising a polynucleotide encoding the activatable HBPC described herein, or a domain thereof. In some aspects, a host cell contains four different vectors, a first vector comprising a first polynucleotide encoding a first polypeptide described herein, a second vector comprising a second polynucleotide encoding a second polypeptide described herein, a third vector comprising a third polynucleotide encoding a third polypeptide described herein, and a fourth vector comprising a fourth polynucleotide encoding a fourth polypeptide described herein.

In some aspects, provided herein is a population of vectors that collectively comprise polynucleotides encoding the first, second, third, and fourth polypeptide, where each vector comprises only one or two of the polynucleotides encoding the first, second, or third polypeptides. In certain aspects, a single vector is provided herein that comprises the polynucleotides encoding the first, second, third, and fourth polypeptides (i.e., the first, second, third, and fourth polynucleotides, respectively).

In some aspects, the present disclosure provides methods of producing an activatable bispecific polypeptide complex comprising: (a) culturing a host cell comprising one or more polynucleotides encoding the polypeptides of the present invention (e.g., a first polynucleotide, a second polynucleotide, a third polynucleotide, and/or a fourth polynucleotide, as well as vector(s) comprising the aforementioned polynucleotides) in a liquid culture medium under conditions sufficient to produce the activatable bispecific polypeptide; and (b) recovering the activatable bispecific polypeptide.

In a particular aspect, provided herein are methods for producing an activatable anti-EGFR, anti-CD3 HBPC, comprising expressing such a polypeptide thereof in a host cell. More specifically, provided herein is a method of producing an activatable bispecific polypeptide complex comprising: (a) culturing a host cell comprising one or more polynucleotides encoding the polypeptides of the present invention in a liquid culture medium under conditions sufficient to produce the bispecific polypeptide; and (b) recovering the activatable bispecific polypeptide.

### Compositions

In some aspects, the activatable HBPCs of the present disclosure or HBPC thereof can be utilized in a pharmaceutical composition useful for any of the therapeutic applications disclosed herein. In certain aspects, the pharmaceutical composition comprises a therapeutically effective amount of one or more activatable HBPC, together with pharmaceutically acceptable diluent or carrier. In other aspect, the pharmaceutical composition comprises a therapeutically effective amount of one or more activatable HBPC, a pharmaceutically acceptable diluent, carrier, solubilizer, emulsifier, preservative, and/or adjuvant. Acceptable formulation materials are nontoxic to recipients at the dosages and concentrations employed. The pharmaceutical compositions can be formulated as liquid, frozen or lyophilized compositions.

In certain aspects, the pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids; antimicrobials; antioxidants; buffers; bulking agents; chelating agents; complexing agents; fillers; carbohydrates such as monosaccharides or disaccharides; proteins; coloring, flavoring and diluting agents; emulsifying agents; hydrophilic polymers; low molecular weight polypeptides; salt-forming counterions (such as sodium); preservatives; solvents (such as glycerin, propylene glycol or polyethylene glycol); sugar alcohols; suspending agents; surfactants or wetting agents; stability enhancing agents; tonicity enhancing agents; delivery vehicles; and/or pharmaceutical adjuvants. Additional details and options for suitable agents that can be incorporated into pharmaceutical compositions are provided in, for example, Remington's Pharmaceutical Sciences, 22^{nd} Edition, (Loyd V. Allen, ed.) Pharmaceutical Press (2013); Ansel et al., Pharmaceutical Dosage Forms and Drug Delivery Systems, 7th ed., Lippencott Williams and Wilkins (2004); and Kibbe et al., Handbook of Pharmaceutical Excipients, 3rd ed., Pharmaceutical Press (2000).

The components of the pharmaceutical composition are selected depending upon, for example, the intended route of administration, delivery format and desired dosage. See, for example, Remington's Pharmaceutical Sciences, 22nd Edition, (Loyd V. Allen, ed.) Pharmaceutical Press (2013). The compositions are selected to influence the physical state, stability, rate of in vivo release and rate of in vivo clearance of the antigen binding proteins disclosed. The primary vehicle or carrier in a pharmaceutical composition can be either aqueous or non-aqueous in nature. For example, a suitable vehicle or carrier can be water for injection or physiological saline solution. In certain aspects, antigen binding protein compositions can be prepared for storage by mixing the selected composition having the desired degree of purity with optional formulation agents in the form of a lyophilized cake or an aqueous solution. Further, in certain aspects, the antigen binding protein can be formulated as a lyophilizate using appropriate excipients.

In certain formulations, the activatable HBPC concentration is at least 2 mg/ml, 5 mg/ml, 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml or 150 mg/ml. In other formulations, the activatable HBPC has a concentration of 10-20 mg/ml, 20-40 mg/ml, 40-60 mg/ml, 60-80 mg/ml, or 80-100 mg/ml.

Some compositions include a buffer or a pH adjusting agent. Representative buffers include, but are not limited to: organic acid salts (such as salts of citric acid, acetic acid, ascorbic acid, gluconic acid, carbonic acid, tartaric acid, succinic acid, or phthalic acid); Tris; phosphate buffers; and, in some instances, an amino acid as described below. In certain aspects, buffers are used to maintain the composition at physiological pH or at a slightly lower pH, typically within a pH range of from about 5 to about 8. Some compositions have a pH from about 5-6, 6-7, or 7-8. In other aspects, the pH is from 5.5-6.5, 6.5-7.5, or 7.5-8.5.

Free amino acids or proteins are used in some compositions as bulking agents, stabilizers, and/or antioxidants. As an example, lysine, proline, serine, and alanine can be used for stabilizing proteins in a formulation. Glycine is useful in lyophilization to ensure correct cake structure and properties. Arginine may be useful to inhibit protein aggregation, in both liquid and lyophilized formulations. Methionine is useful as an antioxidant. Glutamine and asparagine are included in some aspects. An amino acid is included in some formulations because of its buffering capacity. Such amino acids include, for instance, alanine, glycine, arginine, betaine, histidine, glutamic acid, aspartic acid, cysteine, lysine, leucine, isoleucine, valine, methionine, phenylalanine, aspartame, and the like. Certain formulations also include a protein excipient such as serum albumin (e.g., human serum albumin (HSA) and recombinant human albumin (rHA)), gelatin, casein, and the like.

Some compositions include a polyol. Polyols include sugars (e.g., mannitol, sucrose, trehalose, and sorbitol) and polyhydric alcohols such as, for instance, glycerol and propylene glycol, and polyethylene glycol (PEG) and related substances. Polyols are kosmotropic. They are useful stabilizing agents in both liquid and lyophilized formulations to protect proteins from physical and chemical degradation processes. Polyols also are useful for adjusting the tonicity of formulations.

Certain compositions include mannitol as a stabilizer. It is generally used with a lyoprotectant, e.g., sucrose. Sorbitol and sucrose are useful for adjusting tonicity and as stabilizers to protect against freeze-thaw stresses during transport or the preparation of bulk product during the manufacturing process. PEG is useful to stabilize proteins and as a cryoprotectant and can be used in the disclosure in this regard.

Sugars, including monosaccharides, di-, tri-, tetra-, and oligosaccharides; derivatized sugars such as alditols, aldonic acids, esterified sugars and the like; and polysaccharides or sugar polymers can be included in some formulations. For example, suitable carbohydrate excipients include, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like.

Surfactants can be included in certain formulations. Surfactants are typically used to prevent, minimize, or reduce protein adsorption to a surface and subsequent aggregation at air-liquid, solid-liquid, and liquid-liquid interfaces, and to control protein conformational stability. Suitable surfactants include, for example, polysorbate 20, polysorbate 80, other fatty acid esters of sorbitan esters, Triton surfactants, lechithin, tyloxapal, and poloxamer 188.

In some aspects, one or more antioxidants are included in the pharmaceutical composition. Antioxidant excipients can be used to prevent oxidative degradation of proteins. Reducing agents, oxygen/free-radical scavengers, and chelating agents are useful antioxidants in this regard. Antioxidants typically are water-soluble and maintain their activity throughout the shelf life of a product. EDTA is another useful antioxidant.

Certain formulations include metal ions that are protein co-factors and that are necessary to form protein coordination complexes. Metal ions also can inhibit some processes that degrade proteins. For example, magnesium ions (10-120 mM) can be used to inhibit isomerization of aspartic acid to isoaspartic acid.

A tonicity enhancing agent can also be included in certain formulations. Examples of such agents include alkali metal halides, preferably sodium or potassium chloride, mannitol, and sorbitol.

One or more preservatives can be included in certain formulations. Preservatives are necessary when developing multi-dose parenteral formulations that involve more than one extraction from the same container. Their primary function is to inhibit microbial growth and ensure product sterility throughout the shelf-life or term of use of the drug product. Suitable preservatives include phenol, m-cresol, p-cresol, o-cresol, chlorocresol, benzyl alcohol, phenylmercuric nitrite, phenoxyethanol, phenyl alcohol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate, thimerosal, benzoic acid, salicylic acid, chlorhexidine, or mixtures thereof in an aqueous diluent.

A pharmaceutical composition is formulated to be compatible with its intended route of administration. Examples of routes of administration are intravenous (IV), intradermal, inhalation, transdermal, topical, transmucosal, and rectal administration.

Formulation components suitable for parenteral administration (e.g., intravenous, subcutaneous, intraocular, intraperitoneal, intramuscular) include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, N.J.) or phosphate buffered saline (PBS). The carrier should be stable under the conditions of manufacture and should be preserved against microorganisms. The carrier can be a solvent or dispersion medium containing, for example, water, ethanol, polyol (for example, glycerol, propylene glycol, and liquid polyethylene glycol), and suitable mixtures thereof.

Further guidance on appropriate formulations depending upon the form of delivery is provided, for example, in Remington's Pharmaceutical Sciences, 22^{nd} Edition, (Loyd V. Allen, ed.) Pharmaceutical Press (2013).

Pharmaceutical formulations can be sterile. Sterilization can be accomplished by any suitable method, e.g., filtration through sterile filtration membranes. Where the composition is lyophilized, filter sterilization can be conducted prior to or following lyophilization and reconstitution. As demonstrated in Examples 7 and 8, the activatable HBPCs described herein appear relatively aggregation-resistant even at relatively high concentrations. Thus, in another aspect, provided herein are compositions comprising any of the activatable HBPCs described herein, and water, wherein the activatable HBPC is present at a concentration of at least 1 mg/mL and wherein the composition comprises at least about 95% monomeric activatable HBPC, or at least about 96% monomeric activatable HBPC, or at least about 97% monomeric activatable HBPC, or at least about 98% monomeric activatable HBPC, or at least about 99% monomeric activatable HBPC. As used herein, the term “monomeric activatable HBPC” refers to the activatable HBPC in non-aggregated form. In certain of these aspects the composition comprises at least about 2 mg/ml and at least about 95% monomeric activatable HBPC, or at least about 96% monomeric activatable HBPC, or at least about 97% monomeric activatable HBPC, or at least about 98% monomeric activatable HBPC, or at least about 99% monomeric activatable HBPC. In some aspects, the composition comprises at least about 3 mg/ml and at least about 95% monomeric activatable HBPC, or at least about 96% monomeric activatable HBPC, or at least about 97% monomeric activatable HBPC, or at least about 98% monomeric activatable HBPC, or at least about 99% monomeric activatable HBPC. In some aspects, the composition comprises at least about 4 mg/ml and at least about 95% monomeric activatable HBPC, or at least about 96% monomeric activatable HBPC, or at least about 97% monomeric activatable HBPC, or at least about 98% monomeric activatable HBPC, or at least about 99% monomeric activatable HBPC. The percentage of monomeric activatable HBPC can be readily determined by, for example, size exclusion (SE)-HPLC, as illustrated in Example 7, where percent monomeric activatable HBPC is determined as the percentage peak area corresponding to monomeric activatable HBPC on the basis of total peak area.

### EXAMPLES

The examples in this Examples Section are offered by way of illustration, and not by way of limitation.

### Example 1. Construction and Expression of Activatable Anti-EGFR, Anti-CD3 HBPC

An illustrative anti-EGFR, anti-CD3 activatable HBPC, Complex-66, was prepared having the antibody format shown in FIG. 1. With reference to FIG. 1, the activatable HBPC was made up of four polypeptides: (a) a first polypeptide including a CD3 masking moiety (MM1) 100, a first cleavable moiety (CM1) 101, an anti-CD3 scFv 102, an anti-EGFR heavy chain variable domain (top) and a CH1 domain (bottom), together indicated as 103, which is linked, via a hinge region 104, to a first Fc domain 105; (b) a second polypeptide including a EGFR masking moiety (MM2) 106, a second cleavable moiety (CM2) 107, an anti-EGFR light chain variable domain (top), and constant light domain (bottom), together indicated as 108; and (c) a third polypeptide including a CD3 masking moiety (MM3) 109, a first cleavable moiety (CM3) 110, an anti-CD3 scFv 111, an anti-EGFR heavy chain variable domain (top) and a CH1 domain (bottom), together indicated as 112, which is linked, via a hinge region 113, to a second Fc domain 114; and (d) a fourth polypeptide including a EGFR masking moiety (MM4) 115, a second cleavable moiety (CM4) 116, an anti-EGFR light chain variable domain (top) and constant light domain (bottom), together indicated as 117. As shown in FIG. 1, the first and second Fc domains bind each other.

The components used to construct Complex-66 are listed in Tables 3A-3B.

TABLE 3A
Complex-66 First and Third Polypeptide Components

| Name/ | First Polypeptide | | | | |
|---|---|---|---|---|---|
| SEQ ID NO. | MM1 | CM1 | scFv | VH2 | Fc1^{Δ} |
| Polypeptide | ML15 | 0011 | I2C | C225v5 | Fc1 |
| SEQ ID | SEQ ID | SEQ ID | SEQ ID | SEQ ID | SEQ ID |
| NO: 2*^{, ++} | NO: 4 | NO: 5 | NO: 6 | NO: 7 | NO: 28 |

*Corresponding polynucleotide sequence is SEQ ID NO: 1 (the terminal lysine is not present in the purified protein regardless of being present or absent in the gene) or SEQ ID NO: 126.
⁺⁺Contains an N-terminal spacer, SEQ ID NO: 3.
^{Δ}The first Fc domain is located at the C-terminus of a CH1 (SEQ ID NO: 8) − Hinge (SEQ ID NO: 10) sequence.

TABLE 3B
Complex-66 Second and Fourth Polypeptide Components

Second Polypeptide

| | | | | Constant Light |
|---|---|---|---|---|
| Name | MM2 | CM2 | VL2 | Chain (CL) |
| Polypeptide | CF41 | 2008 | C225v5 | CL |
| SEQ ID | SEQ ID | SEQ ID | SEQ ID | SEQ ID |
| NO: 16*^{, ++} | NO: 18 | NO: 19 | NO: 20 | NO: 21 |

*Corresponding polynucleotide sequence is SEQ ID NO: 15.
⁺⁺Contains an N-terminal spacer, SEQ ID NO: 17.

### Construction of Control Anti-EGFR, Anti-CD3 Activatable HBPC Antibody

The control activatable bispecific construct was an activatable dual-armed divalent bispecific construct, referred to herein as “CI106,” which was prepared as described in international patent application Pub. No. WO 2019/075405, which is incorporated herein by reference. CI106 is made up of four polypeptides corresponding to two identical heavy chains and two identical light chains, where each heavy and light chain form an arm of the bispecific construct. Each heavy chain encodes, from N-terminus to C-terminus, a first polypeptide including a CD3 masking moiety (MM1), a first cleavable moiety (CM1), an anti-CD3 scFv, an anti-EGFR heavy chain variable domain, and a first Fc domain. Each light chain encodes, from N-terminus to C-terminus, a second polypeptide including an EGFR masking moiety (MM2), a second cleavable moiety (CM2), and an anti-EGFR light chain variable domain. The amino acid sequence of the light chain of CI106 is identical to the amino acid sequence of the second polypeptide of Complex-66. The heavy chain of CI106 and the first polypeptide of Complex-66 have identical spacer, CM/CM1, anti-EGFR VL, anti-EGFR VH, anti-EGFR MM/MM2, and CM/CM2 components. However, the heavy chain of CI106 and the first polypeptide of Complex-66 have different anti-CD3 light chain variable regions, anti-CD3 heavy chain variable regions, and anti-CD3 MM1/MM3 components. The components used to construct CI106 are provided in Tables 4A-4B.

TABLE 4A
CI106 Heavy Chain

| | Heavy Chain | | | | |
|---|---|---|---|---|---|
| | | | | Anti-EGFR | |
| | Anti-CD3 | | | Variable | CH1 + |
| | Masking | Cleavable | Anti-CD3 | Heavy | Hinge + |
| Name | Moiety | Moiety | scFv | Domain | Fc ^{Δ} |
| CI106 HC | H20GG | 0011 | v16 | C225v5 | SEQ ID |
| SEQ ID | SEQ ID | SEQ ID | SEQ ID | SEQ ID | NO: 29 |
| NO: 27*^{, ++} | NO: 24 | NO: 5 | NO: 25 | NO: 7 | |

*Corresponding polynucleotide sequence is SEQ ID NO: 26.
⁺⁺Contains an N-terminal spacer (SEQ ID NO: 3).
^{Δ} The Fc domain is located at the C-terminus of a CH1 (SEQ ID NO: 8) − Hinge (SEQ ID NO: 10) sequence.

TABLE 4B
CI106 Light Chain

| | | | Light Chain | |
|---|---|---|---|---|
| | Anti-EGFR | | | Constant |
| | Masking | Cleavable | Anti-EGFR | Light |
| Name | Moiety | Moiety | VL | Chain |
| CI106 LC | CF41 | 2008 | C225v5 | CL |
| SEQ ID | SEQ ID | SEQ ID | SEQ ID | SEQ ID |
| NO: 16*^{, ++} | NO: 18 | NO: 19 | NO: 20 | NO: 21 |

*Corresponding polynucleotide sequence is SEQ ID NO: 15.
⁺⁺Contains an N-terminal spacer, SEQ ID NO: 17.

### Example 2. Binding of Activatable HBPCs to EGFR⁺ HIT-29 Cells and CD3ε⁺ Jurkat Cells

A flow cytometry-based binding assay was performed to confirm that the described anti-EGFR and anti-CD3 masking peptides could inhibit binding of an activatable (masked) HBPC to EGFR and CD3.

HT-29-luc2 (Perkin Elmer, Inc., Waltham, MA (formally Caliper Life Sciences, Inc.) and Jurkat (Clone E6-1, ATCC, TIB-152) cells were cultured in RPMI-1640+glutamax (Life Technologies, Catalog 72400-047) supplemented with 10% Heat Inactivated-Fetal Bovine Serum (HI-FBS, Life Technologies, Catalog 10438-026). As indicated, “activated” (or “act-”) molecules were produced as masked HBPC and proteolytically cleaved to produce the activated forms. The activatable HBPC were produced as masked antibodies but not subjected to proteolytic cleavage prior to experimentation.

The following HBPCs were tested: (1) activated (unmasked) CI106 (as described in WO 2019/075405, which is incorporated herein by reference) and (2) Complex-66 (activatable/masked).

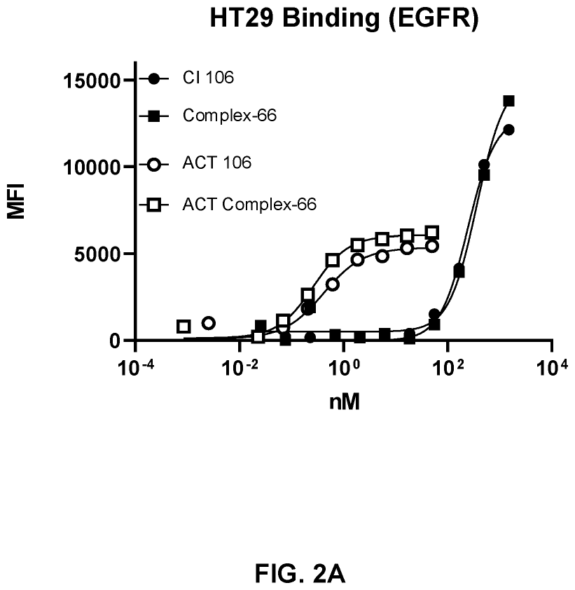

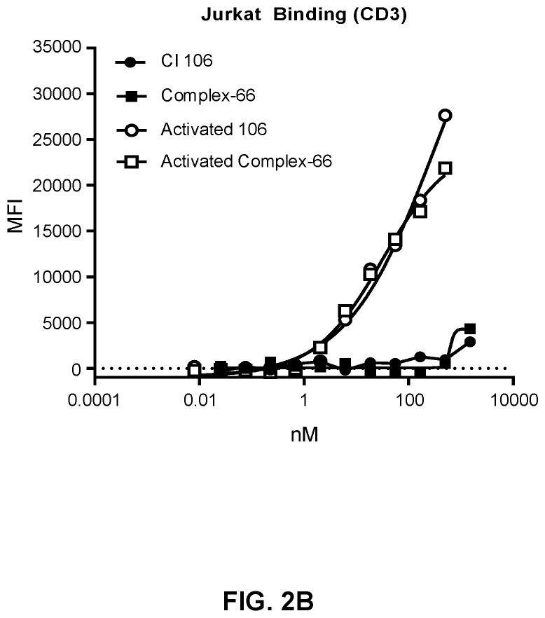

HT29-luc2 cells were detached with Versene™ (Life Technologies, Catalog 15040-066), washed, plated in 96 well plates at approximately 150,000 cells/well, and re-suspended in 50 μL of activated or activatable HBPC. Jurkat cells were counted and plated as described for HT29-luc2 cells. Titrations of activated (mask was proteolytically cleaved) or activatable HBPCs started at the concentrations indicated in FIGS. 2A and 2B followed by 3-fold serial dilutions in FACS Stain Buffer+2% FBS (BD Pharmingen, Catalog 554656). Cells were incubated at 4° C. with shaking for about 1 hour, harvested, and washed with 2×200 μL of FACS Stain Buffer. Cells were resuspended in 50 μL of Alexa Fluor 488 conjugated anti-Human IgG Fc (10 μg/ml, Jackson ImmunoResearch) and incubated at 4° C. with shaking for about 1 hour. Cells were harvested, washed, and resuspended in a final volume of 200 μL of FACS Stain Buffer containing 2.5 μg/mL 7-AAD (BD Biosciences, Catalog 559925). Cells stained with secondary antibody alone were used as a negative control. Data was acquired on an Attune NxT Flow Cytometer and the median fluorescence intensity (MFI) of viable cells was calculated using FlowJo® V10 (Treestar). Background subtracted MFI data was graphed in GraphPad Prism using curve fit analysis.

Reduced binding to both EGFR and CD3 for Complex-66 (activatable HBPC) and CI106, relative to the activated (unmasked) Complex-66 and activated (unmasked) CI106, are shown in FIGS. 2A-2B, as represented by a right shift of the binding curves. The results indicate that the masking moieties for the EGFR-targeting domains in Complex-66 effectively impair binding of the EGFR-targeting domain to EGFR (FIG. 2A) and the masking moieties for the CD3-targeting domains effectively impair binding of the CD3-targeting domain to CD3 (FIG. 2B). The results further indicate that binding can be effectively restored following activation.

### Example 3. Biological Activity of HBPCs

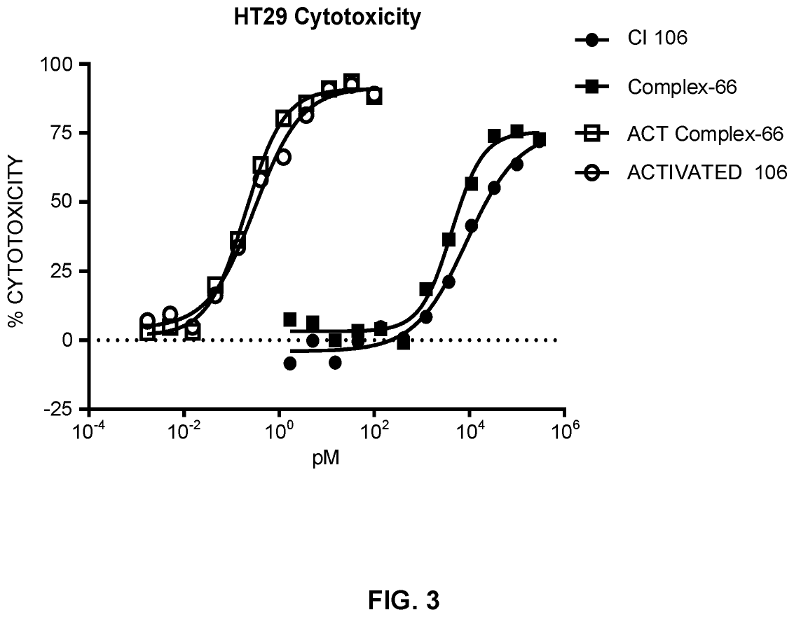

Biological activity of activatable and activated HBPCs was assayed using cytotoxicity assays. Human PBMCs were purchased from Stemcell Technologies (Vancouver, Canada) and co-cultured with EGFR expressing cancer cell line HT29-luc2 (Perkin Elmer, Inc., Waltham, MA (formally Caliper Life Sciences, Inc.)) at an E (CD3+): T ratio of 5:1 in RPMI-1640+glutamax supplemented with 5% heat inactivated human serum (Sigma, Catalog H3667). Titrations of activated CI106 (control), activated Complex-66, CI106, and Complex-66 were tested. After 48 hours, cytotoxicity was evaluated using the ONE-Glo™ Luciferase Assay System (Promega, Madison, WI Catalog E6130). Luminescence was measured on the Infinite® M200 Pro (Tecan Trading AG, Switzerland). Percent cytotoxicity was calculated and plotted in GraphPad PRISM with curve fit analysis. Potency of the activated molecules was compared by calculating the EC50 ratios. As shown in FIG. 3, the activatable HBPCs have a shifted dose response curve relative to the activated molecules. The results indicate that the masking moieties for the CD3-targeting domains in Complex-66 effectively impair binding of the CD3-targeting domains to CD3. The results further indicate that binding can be effectively restored following activation.

### Example 4. HBPC Induced Regression of Established HT29 Tumors in Mice

Activatable (masked) HBPCs Complex-66 and CI106 were analyzed for the ability to induce regression of or reduce the growth of established HT29 xenograft tumors in human PBMC engrafted NSG mice.

The human colon cancer cell line HT29-luc2 (Perkin Elmer, Inc., Waltham, MA) was cultured according to established procedures. Purified, frozen human PBMCs were obtained from Hemacare, Inc. (Van Nuys, CA). NSG (NOD.Cg-Prkdcscid II2rg^{tmIWjl}/SzJ) mice were obtained from The Jackson Laboratories (Bar Harbor, ME).

On day 0, each mouse was inoculated subcutaneously at the right flank with 2×10⁶ HT29-luc2 cells in 100 μL RPMI+Glutamax, serum-free medium. Previously frozen PBMCs from a single donor were administered (i.p.) on day 3 at a CD3⁺ T cell to tumor cell ratio of 1:1. When tumor volumes reached 150-200 mm³ (approximately day 12), mice were randomized, assigned to treatment groups and dosed i.v. according to Table 5. Tumor volume and body weights were measured twice weekly.

TABLE 5
Groups and Doses for HT29-luc2 Xenograft Study

| Treatment Group | Dose, mpk (mg/kg) |
|---|---|
| Vehicle | 0 |
| CI106 | 1.0 |
| Complex-66 | 0.3 |
| Complex-66 | 1.0 |
| Complex-66 | 3.0 |

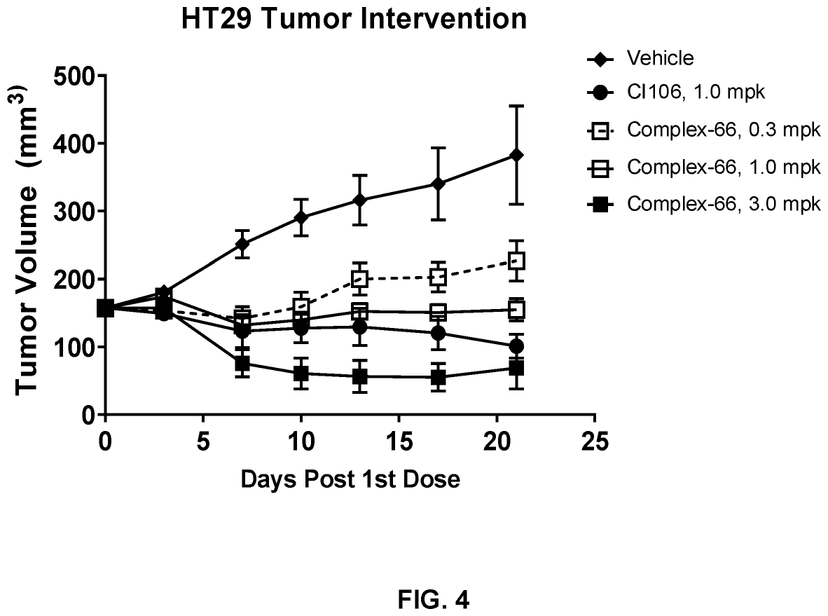

As shown in FIG. 4, a dose-dependent effect of Complex-66 on the growth of HT29-luc2 xenograft tumors was observed Complex-66 demonstrated similar anti-tumor activity as CI106, at the equivalent dose (1 mg/kg); p=0.0099 RMANOVA with Dunnett's).

### Example 5. Tumor Regression of Established HCT116 Tumors in Mice Following Treatment with Activatable HBPCs

Activated (unmasked) Complex-66, and masked Complex-66 were analyzed for the ability to induce regression of or reduce the growth of established HCT116 xenograft tumors in human T-cell engrafted NSG mice. The human colon cancer cell line HCT116 (ATCC) was cultured in RPMI+Glutamax+10% FBS according to established procedures. The tumor model was carried out as described in Example 4. Mice were dosed according to Table 5.

TABLE 6

| Treatment Group | Dose, mpk |
|---|---|
| Vehicle | 0 |
| Act-Complex-66 | 0.3 |
| Act-Complex-66 | 1 |
| Complex-66 | 1 |
| Complex-66 | 3 |

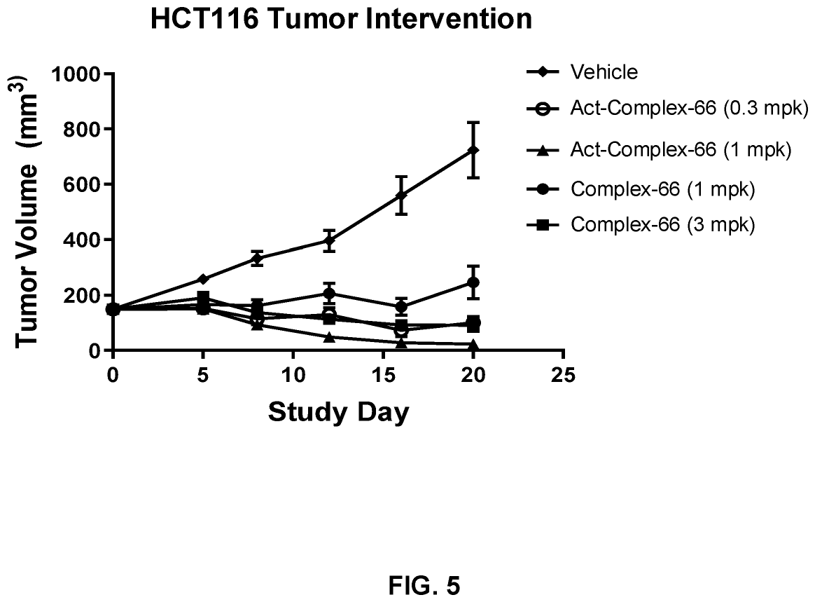

As shown in FIG. 5, tumor regression for both molecules was demonstrated at all doses tested, thus confirming that Complex-66 was successfully activated.

### Example 6. Cross Reactivity of Dually Masked Multispecific, Activatable Antibodies to Cynomolgus Monkey

To confirm that Cynomolgus monkey is a relevant toxicity species, CI106, Complex-66 and activated Complex-66 were used in flow cytometry based cell binding assays and a cytotoxicity assay using target cells expressing cyno EGFR (CHO cEGFR) and cynomolgus pan T cells or cynomolgus PBMC (BioreclamationIVT). Methods are as described in Examples 2 and 3.

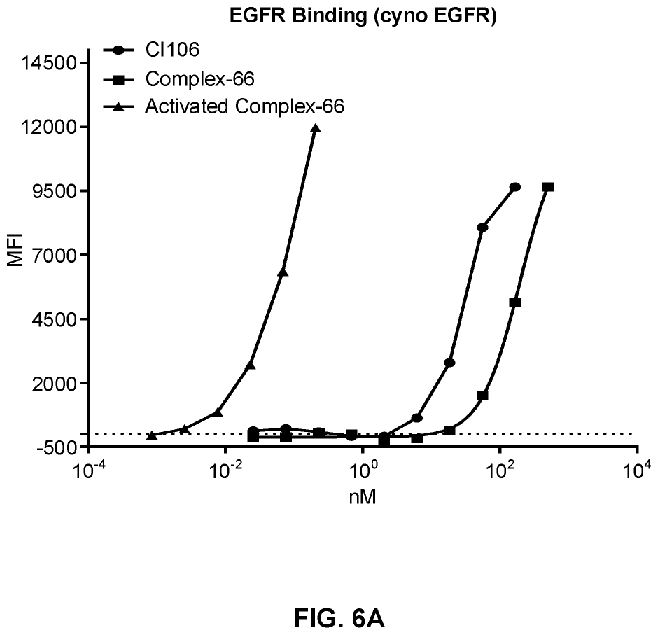

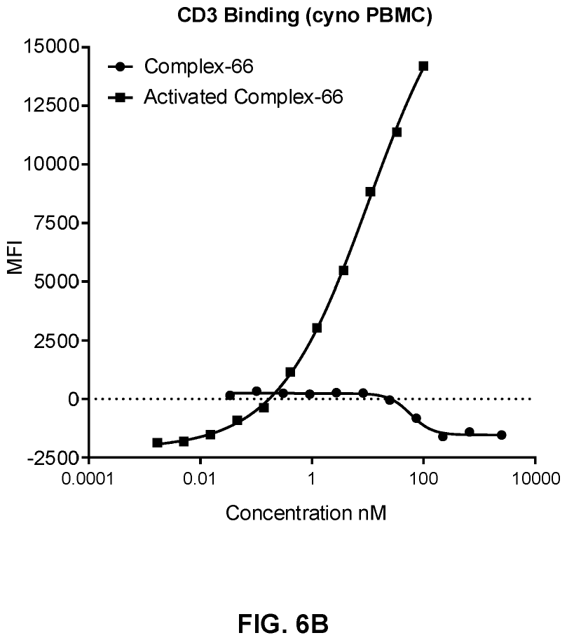

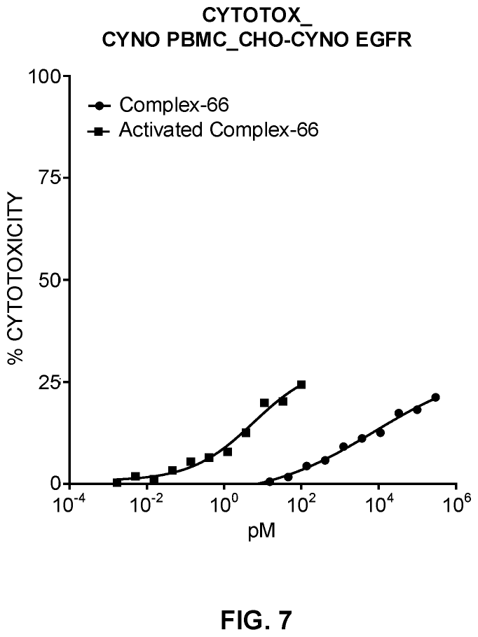

FIGS. 6A-6B and 7 demonstrate that activated Complex-66 binds to cyno EGFR (FIG. 6A) and cyno CD3 (FIG. 6B) and that the dually masked molecules have a shifted binding curve relative to the activated molecules. FIG. 7 demonstrates functional cytotoxic activity against target cells expressing cyno EGFR and using cyno effector cells. Therefore, cynomolgus monkey was determined to be a relevant species for toxicity studies.

### Example 7: Evaluation of Percent Monomer after Purification with Ceramic Hydroxyapatite Chromatography (CHT)

The CI106 (control) and Complex-66 (activatable HBPC) were purified using a ceramic hydroxyapatite chromatography column to compare the amount of dimerization at high concentrations during purification. This was assessed by analyzing the percentage of monomer at each step in the purification process.

Samples were loaded on a CHT Type I, 40 μm bead column (Biorad Cat: 157-0040 and #157-0041) loaded at 20 g/L resin. The column was washed with equilibration buffer 10 mM NaPO4, 100 mM Histidine buffer pH 6.5, and then eluted in 2 mL fractions with 10 mM NaPO4, 100 mM Histidine 200 mM Lysine-HCl buffer at pH 6.5 for CI106 and 10 mM NaPO4, 100 mM Histidine 100 mM Lysine-HCl buffer at pH 6.5 for Complex-66. CI106 was collected in 2 mL fractions and then five fractions were pooled to form the eluate. Peak collection started around 25 mAU and stopped around 300 mAU for CI106. Complex-66 was collected in one tube, with peak collection starting at 100 mAU and stopping at 500 mAU. This was followed by a strip buffer step of 500 mM NaPO4 at pH 7.0. Protein concentration for each fraction was quantified by UV absorption at a wavelength of 280 nm. The percent monomer in each fraction was determined by SE-HPLC (Analytical scale size exclusion chromatography), on the basis of total peak area. During the binding stage of chromatography, protein binds first to the top portion of the column and only moves down the column as the upper sites become full. This causes the molecules to be at a high concentration on the column. The multimeric forms of CI106 and Complex-66 bind with a stronger affinity to the column than the monomeric forms and therefore require a stronger buffer for complete removal from the column. Therefore, when the column is eluted with a weaker buffer and then stripped with a stronger buffer, the eluates have a lower percentage of dimer (higher percentage of monomer) than the strips. Table 7 shows that the Complex-66 run resulted in an increase in percent monomer of 14.0% in the eluate, leaving the high molecular weight material on the column until the strip step, which lead to a 73% recovery of monomer in the eluate. By contrast, the CI106 run resulted in a decrease in percent monomer by 5.4% in the eluate to 65.0%, even though more dimeric material (only 30.6% monomer) stayed on the column until the strip, resulting in 81% recovery in the eluate.

TABLE 7
CHT Chromatography Results

| | % monomer | % Monomer | % Recovery | % Monomer | % Recovery |
|---|---|---|---|---|---|
| Molecule | in load | in eluate | in eluate | in strip | in strip |
| Complex-66 | 74.9 | 88.9 | 73 | 39.0 | 23 |
| CI106 | 70.4 | 65.0 | 81 | 30.6 | 9 |

These results suggest that Complex-66 does not undergo additional dimerization when at a high concentration on the column, resulting in removal of almost all of the high molecular species (88.9% monomer in the eluate compared to only 65% for CI106). For CI106 there are more high molecular species in the eluate pool than the original load. However, CI106 cannot be purified by CHT chromatography, or any bind/elute chromatography method, due to the dimerization that occurs when CI106 is subjected to high concentrations on the column. The improved behavior of Complex-66 enables purification of high monomeric Complex-66 via CHT type 1 chromatography.

### Example 8: Assessment of Concentration Dependent Dimerization via Concentrating in a Centrifugal Concentrator

Protein A and SEC-purified preparations of Complex-66 and CI106 (control) were compared for percent monomer concentration after centrifugal concentration and overnight incubations at the highest concentration.

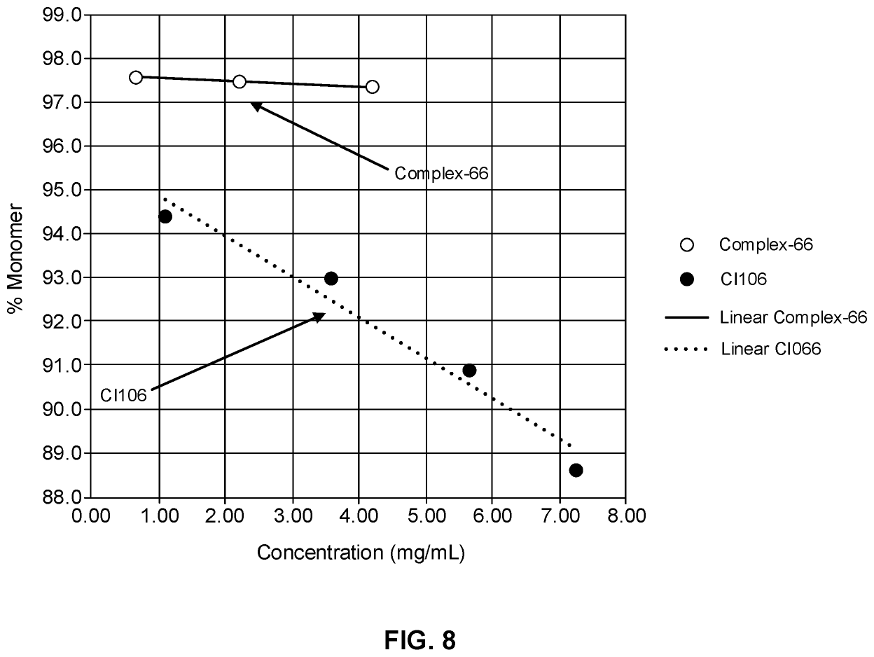

Complex-66 and CI106 (control) were purified with Protein A and SEC and then formulated in a low pH buffer (10 mM acetate, 100 mM lysine, pH 6). Samples were diluted 1:15 into PBS (753-45-01) and concentrated using Pierce™ Protein Concentrators PES 10K MWCO 0.5 ml (Thermo Fisher cat. #88513) by centrifuging 14,000 RPM for 2 minutes at each concentration. The highest concentrated samples were stored overnight and assessed for percent monomer. The resulting concentrations and percent monomer amounts are shown in Table 8 and FIG. 8. Percent monomer was determined as described in Example 7.

TABLE 8
Percent Monomeric Activatable HBPC
vs. Total Protein Concentration

| | |
|---|---|
| Complex-66 | CI106 in PBS |

| Conc. mg/ml | % Monomer | Conc. mg/ml | % Monomer |
|---|---|---|---|
| 1.0 | 97.6 | 1.1 | 94.4 |
| 3.2 | 97.5 | 3.58 | 93.0 |
| 5.4 | 97.3 | 5.67 | 90.9 |
| 6.5 | | 7.27 | 88.6 |

As shown in FIG. 8 and Table 8, Complex-66 maintains a high percentage of monomer in solution as concentration is increased. In contrast, CI106 shows marked concentration dependent dimerization as concentration is increased. Complex-66 also maintained percentage monomer concentration during an overnight incubation at the highest concentration, demonstrating the stability of the monomer at concentration.

The results suggest that Complex-66 is less prone to aggregation and less prone to concentration-dependent aggregation as compared to CI106.

### Example 9: Safety and Efficacy of anti-EGFR, anti-CD3 TCB Construct CI107

In this study, the safety and efficacy of CI107, an anti-EGFR, anti-CD3 TCB construct having the same structural format of the CI106 control (described above), was evaluated in preclinical models to assess the therapeutic potential for the treatment of EGFR-expressing tumors. CI107 was prepared as described in international patent application Pub. No. WO 2019/075405, which is incorporated herein by reference. The CI107 TCB construct is alternatively referred to in this example as a “T cell-engaging bispecific antibody” or “TCB.”

### Methods

### Animal Studies

All animal studies were performed in accordance with the Institutional Animal Care and Use Committee regulations governing the facility that performed each study. Mouse xenograft studies were performed by CytomX Therapeutics, Inc (CytomX), and cynomolgus monkey studies were performed by Altasciences (Everett, WA). All animal studies followed regulations set forth by the USDA Animal Welfare Act and the Guide for the Care and Use of Laboratory Animals.

### Materials

All TCBs and other constructs described in this study, including CI107, CI128, CI020, CI011, CI040, CI048, and CI104, were generated by CytomX Therapeutics, Inc. (see, WO 2016/014974 and WO 2019/075405). CI107, CI128, CI020, CI011, CI040, and CI104 have the same structural format as CI106. CI048 corresponds to activated CI011. Activated TCBs were generated by in vitro treatment with urokinase-type plasminogen activator (uPA) followed by SEC purification (Desnoyers 2013). HT29-Luc2 cells were obtained from Caliper Life Sciences (Hopkinton, MA), and HCT116 and Jurkat cells were obtained from American Type Culture Collection (ATCC). Human peripheral blood mononuclear cells (PBMCs) were obtained as cryopreserved vials of cells from individual donors from HemaCare Corporation (Northridge, CA), AllCells (Alameda, CA), or STEMCELL Technologies (Seattle, WA). NOD.Cg-Prkcdscid Il2rg tm1Wjl/SzJ (NSG) mice were obtained from Jackson Laboratories (Sacramento, CA).

### Cell Binding Assays

HT29 and Jurkat cells were maintained in complete media. HT29 cells were harvested using Versene™ cell dissociation buffer. Cells were centrifuged at 250×g for 5-10 minutes and resuspended in FACS buffer containing 2% FBS (BD Pharminogen). Cells were plated at 150,000/well in V-bottom 96-well plates and treated with CI107 or in vitro protease-activated CI104 at various concentrations obtained by 3-fold serial dilutions in FACS buffer, starting at 1.5 μM CI107 for both HT29 and Jurkat cells, 0.05 μM activated CI104 for HT29 cells, and 0.5 μM activated CI104 for Jurkat cells. Cells were incubated for 1 hour at 4° C., washed twice with FACS buffer, and resuspended in 10 μg/ml Alexa Fluor 647 anti-human Fc secondary antibody. The cells were then incubated, protected from light, for 30-60 minutes at 4° C., washed twice with FACS buffer, resuspended in FACS buffer containing 7-AAD, and analyzed on a MACSQuant flow cytometer (Miltenyi Biotech). Mean fluorescence intensity data were corrected for secondary antibody background signal, graphed in Graphpad Prism, and EC50 values were calculated.

### Cytotoxicity Assays

HCT116-Luc2 or HT29-Luc2 were plated into a 96-well white, flat-bottom, tissue culture-treated plate (Costar #3917) at 10,000 cells/well in RPMI+5% human serum. Human PBMCs were freshly thawed and washed twice with RPMI+5% human serum, and 100,000 PBMCs were added in RPMI+5% human serum to the wells containing HCT116-Luc2 or HT29-Luc2. Protease-activated TCB or CI107 was then added to the wells at various concentrations obtained by 3-fold serial dilutions. Control wells contained untreated target+effector cells, target cells only, effector cells only, or media only. The plates were then incubated at 37° C. and 5% CO2 for approximately 48 hours. Cell viability was measured using the ONE-Glo Luciferase Assay System (Promega, #E6120) and a Tecan plate reader. The percent cytotoxicity was calculated as follows: (1-(RLU experimental/average RLU untreated))*100.

### In Vitro T Cell Activation and Cytokine Analysis

T cell activation was measured by induction of CD69 expression in PBMCs co-cultured with HT29-Luc2 or HCT116-Luc2 cells. HT29-Luc2 or HCT116-Luc2 cells were plated at 10,000 cells/well in a U-bottom non-adherent plate. Human PBMCs were freshly thawed and washed twice with RPMI containing serum, and 100,000 PBMCs/well were added to the plates containing tumor cells. Duplicate plates containing PBMCs only were seeded for flow cytometry compensation controls. Three-fold serial dilutions of CI107, activated CI107, or CI128 were prepared in media and added to the plated cells. Cells were incubated at 37° C. and 5% CO₂ for 16 hours. To prepare for flow cytometry analysis, plates were centrifuged at 250×g for 10-15 minutes. The supernatant was removed for cytokine analysis, Fc block (Human TruStain FcX, BioLegend) was added to each well, and the plates were incubated for 10 minutes. Antibody cocktails containing anti-CD45-FITC (BioLegend), anti-CD3-Pacific blue (BioLegend), anti-CD8a-APC (BioLegend), and anti-CD69-PE-Cy7 (BioLegend), or appropriate compensation controls were added to the wells, and the plates were incubated with shaking at 4° C. protected from light for 30-60 minutes. The plates were then washed with FACS buffer and resuspended in FACS buffer containing 7-AAD. Fluorescence was measured using an Attune Flow Cytometer, and 15,000 events representing PBMCs were collected.

For cytokine analysis, Meso Scale Discovery U-PLEX plate assays (Meso Scale Diagnostics, Rockville, MA) were used. U-PLEX plates were prepared following the manufacturer's protocol to evaluate levels of MCP-1, TNF-α, IL-6, IL-2, and IFN-γ. Supernatant samples collected from HT29-Luc2 or HCT116-Luc2 co-cultured with PBMCs and treated with masked (activatable) CI107, activated (also referred to herein as “act-”) CI107, or CI128 were diluted, added to the plate, and processed following the manufacturer's instructions.

### In Vivo Efficacy Studies

For in vivo experiments, effects of TCBs on tumor growth were measured in mice harboring HT29-Luc2 or HCT116 tumors and engrafted with human T cells resulting from intraperitoneal (IP) injection of human PBMCs. Two million HT29-Luc2 or HCT116 cells were subcutaneously injected in 100 μl serum-free RPMI into the flank of female NSG mice on Day 0. Frozen PBMCs from a single donor were freshly thawed and administered via IP injection on Day 3 in 100-200 μL RPMI+Glutamax, serum-free medium. PBMCs were previously characterized for CD3+ T cell percentage, and the number of PBMCs to be used for in vivo administration was based on a CD3+ T cell to tumor cell ratio of 1:1. Tumor measurements on approximately Day 12 were used to randomize mice prior to intravenous (IV) dosing with TCB, control article, or vehicle. Animals were dosed weekly for 3 weeks with test articles, and tumor volumes and body weights were recorded twice weekly. Activated TCBCI104 was used for in vivo studies. The CI104 construct differs from CI107 only in the cleavable linker used to tether the CD3 mask to the scFv. Upon in vitro protease activation to fully remove the masks, activated CI104 is identical to activated CI107 and can be used to assess the activity of activated CI107, and subsequent in vitro cytotoxicity studies validated that the activity of activated CI104 is the same as that of activated CI107.

### Non-Human Primate Safety Studies

Male cynomolgus monkeys received slow IV bolus injection of test articles on Day 1 or once on Days 1 and 15, depending on the test article. Following test article administration, clinical observations were performed twice daily. Blood samples were collected at various time points post-dose for analysis of cytokine release, serum chemistry, hematology, and toxicokinetics. Cytokine analysis was performed on serum samples using the Life Technologies Monkey Magnetic 29-Plex Panel Kit (Thermo Fisher Scientific, Waltham, MA). For toxicokinetic analysis, samples were processed to plasma and stored at −60 to −86° C. prior to shipment for analysis by AIT Bioscience (Indianapolis IN) or CytomX. Plasma concentrations of test articles were measured by ELISA using an anti-idiotype capture antibody and an anti-human IgG (Fc) capture antibody. Toxicokinetic analysis was performed by Northwest PK Solutions using a noncompartmental analysis utilizing Phoenix WinNonlin v6.4 (Certara, Princeton, NJ).

### Results

CI107 was designed as a dual-masked tetravalent bispecific molecule containing anti-EGFR and anti-CD3 domains. CI107 was generated using a cetuximab-derived antibody with an SP34-derived anti-CD38 scFv fused to the N terminus of the heavy chain. CI107 has a human IgG1 Fc domain with mutations that silence Fc function. To generate CI107, a specific masking peptide for the anti-EGFR antibody component was fused to the N terminus of the light chain using a protease-cleavable substrate linker flanked by flexible Gly-Ser-rich peptide linkers, as previously described (Desnoyers 2013). A masking peptide specific for the anti-CD3 component was similarly added to the scFv using a protease-cleavable substrate linker. CI107 impaired Fc-effector function to minimize cross-linking to cells expressing FcγR. The design is intended to maximize target binding and activity in the protease-rich tumor microenvironment while minimizing binding and activity in normal tissues. All of the comparative TCBs used throughout this example contain EGFR and CD3 binding domains, masks, and linker peptides with varying degrees of cleavability. CI011 and CI040 are first generation versions of CI104 and CI107. The CI104 and CI107 molecules contain an optimized CD3 scFv, next generation cleavable linkers, and additional Fc silencing mutations. CI104 and CI107 have the same masks and EGFR and CD3 binding domains, but differ in the CD3 protease linker; however, after protease activation, the activated TCB is the same. CI128 was used as a non-targeted control in which the EGFR binder is replaced by an irrelevant antibody (anti-RSV).

### Masking Impairs Binding to EGFR on the Cell Surface.

To assess whether masking of the EGFR binding domain impairs binding to EGFR expressed on the cell surface, the binding of CI107 and comparative activated TCB constructs (i.e., act-TCBs) to EGFR-expressing HT29 and HCT116 cells was measured.

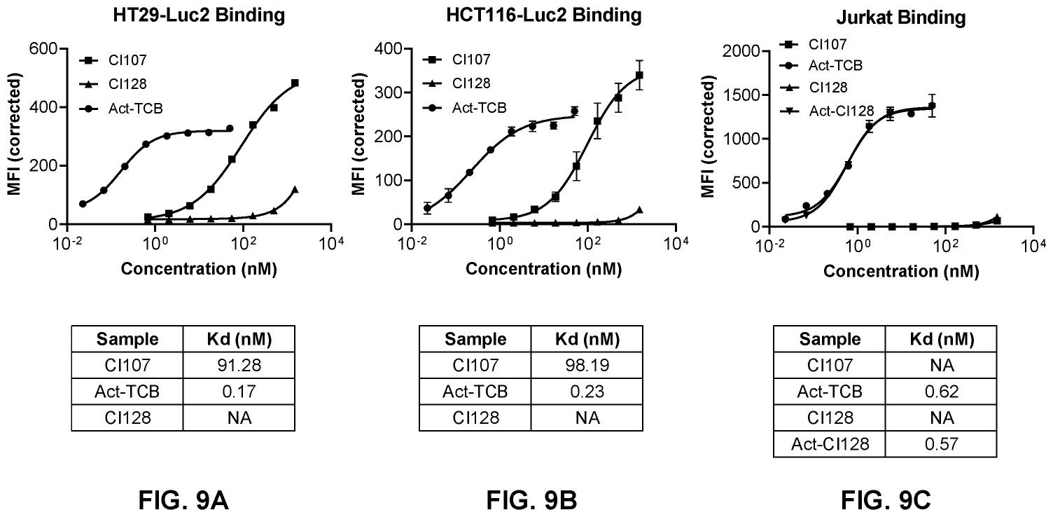

Target cells were incubated with increasing concentrations of CI107 or comparative activated constructs, and binding was evaluated by flow cytometry. As shown in FIG. 9A, the presence of the EGFR mask in CI107 substantially attenuated binding to EGFR expressed on the cell surface compared with activated TCB CI107. Activated TCB constructs bound to HT29 cells with a calculated K_{d} of 0.17 nM, whereas the K_{d} for binding of CI107 was 91.28 nM, representing a greater than 500-fold decrease in binding compared to activated TCB. Similar results were obtained using HCT116 cells (FIG. 9B). Binding of CI128, an untargeted control TCB which contains the same anti-CD3 module as CI107 but lacks EGFR targeting was also evaluated. This control did not bind to HT29 or HCT116 cells (see FIGS. 9A and 9B, respectively).

### Masking Impairs Binding to CD3 on the Surface of Lymphocytes.

To determine whether masking of the anti-CD3 binding domain impairs binding of CI107 to CD3 on the surface of lymphocytes, CI107 and activated CI107 (i.e., activated TCB) binding to Jurkat cells was measured. As shown in FIG. 9C, activated TCB bound to Jurkat cells with a K_{d} of 0.62 nM. However, binding of CI107 was not detected, and a K_{d} could not be calculated. Activated control CI128 bound Jurkat cells with similar affinity as activated TCB.

Together, these data demonstrate that dual masking of anti-EGFR and anti-CD3 binding domains in CI107 attenuates binding to cells expressing EGFR or CD3.

### Masking Attenuates Cytotoxicity and T Cell Activation in PBMCs Co-Culture.

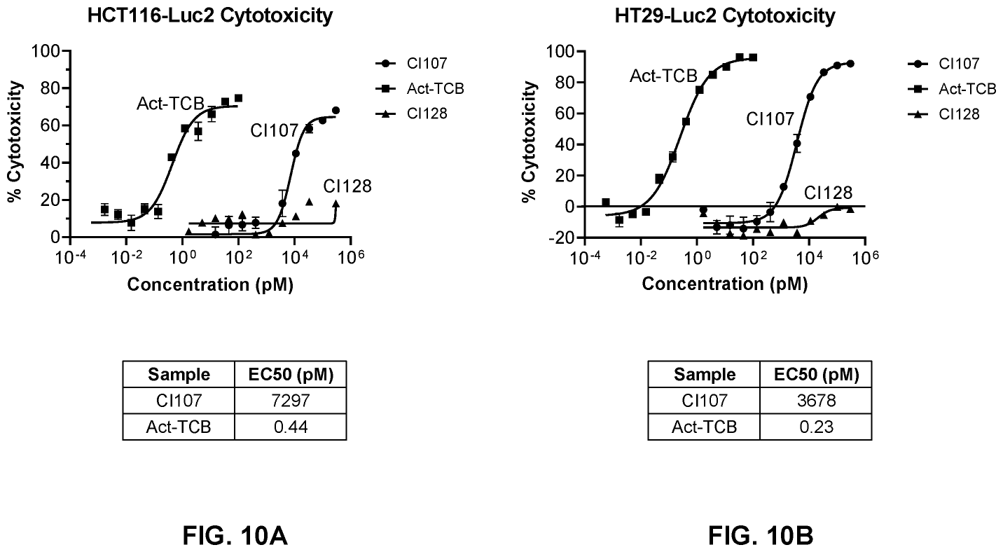

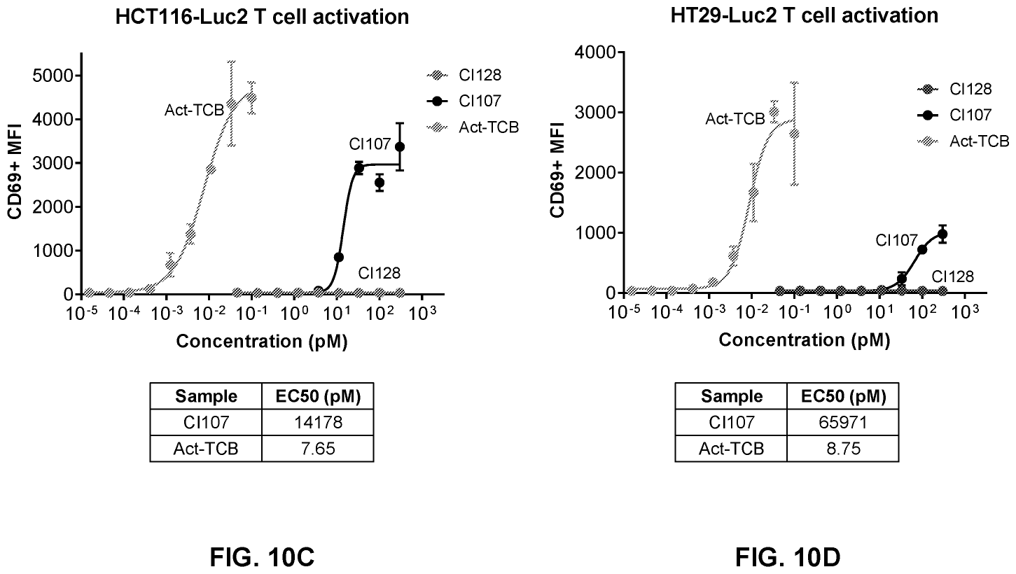

To address whether targeting EGFR with CI107 could lead to anti-tumor cell effects, in vitro cytotoxicity assays were performed. Luciferase-expressing HT29 or HCT116 cells were co-cultured with human PBMCs and incubated with increasing concentrations of CI107, activated TCB, or the untargeted control CI128. After 48 hours of culture, viability of the HCT116-Luc2 or HT29-Luc2 cells was measured via luciferase assay. As shown in FIG. 10A, treatment with the control CI128 resulted in minimal cytotoxicity to HCT116-Luc2 cells co-cultured with PBMCs, demonstrating that engagement of both EGFR and CD3 is required for cytotoxic activity. In contrast, both masked CI107 and activated CI107 (i.e., act-TCB) had cytotoxic effects on HCT116-Luc2 cells. However, activated TCB resulted in cytotoxicity at much lower concentrations compared with the masked form, with EC50 values of 0.44 PM and 7297 pM, respectively. Similar results were observed in HT29-Luc2 cells, with EC50 values of 0.25 pM for activated TCB vs. 3678 pM for CI107 (FIG. 10B). Therefore, dual masking of the anti-EGFR and anti-CD3 domains in CI107 resulted in an approximately 15,000-fold decrease in cytotoxic activity mediated by PBMCs in the absence of protease activation.

Treatment with CI107 Results in Induction of CD69 Expression, a Marker of T Cell Activation.

To determine whether CI107 results in T cell activation, CD69 levels in PBMCs co-cultured with HCT116-Luc2 or HT29-Luc2 cells were measured after treatment with masked CI107, activated CI107 (i.e., act-TCB), and control CI128. CD69 acts as a marker of T cell activation; after TCR/CD3 engagement, CD69 expression is rapidly induced on the surface of T lymphocytes and acts as costimulatory molecule for T cell activation and proliferation. Human PBMCs co-cultured with HCT116-Luc2 or HT29-Luc2 cells were treated with increasing concentrations of CI107, activated TCB (i.e., activated CI107), or control CI128 for 16 hours, and CD69 expression levels were measured by flow cytometry. As shown in FIG. 10C, CI107 resulted in induction of CD69 expression on CD8+ T cells cocultured with HCT116-Luc2 cells with an EC50 of 14178 pM. In contrast, treatment with activated CI107 resulted in CD69 induction with an EC50 of 7.65 pM, reflecting an approximately 18,000-fold shift in the T cell activation curve compared with CI107. T cell activation was not observed with the non-EGFR targeted control CI128, indicating that engagement of CD3 alone is not sufficient for T cell activation. Similarly, treatment of PBMCs from the same donor co-cultured with HT29-Luc2 cells resulted in CD69 induction with EC50 values of 65971 pM for masked CI107 vs. 8.75 pM for activated TCB, reflecting an approximately 7500-fold difference in CD69 induction capacity (FIG. 10D).

Treatment with CI107 Results in Cytokine Release.

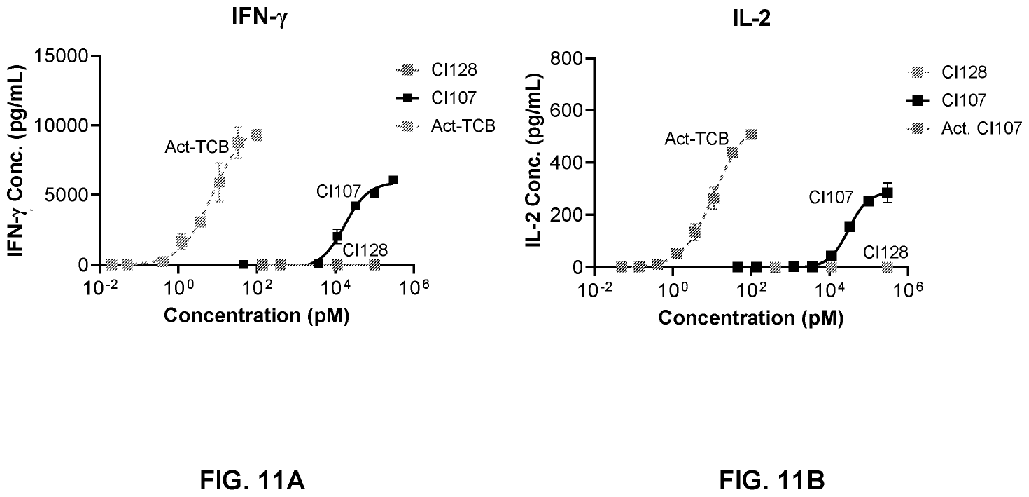

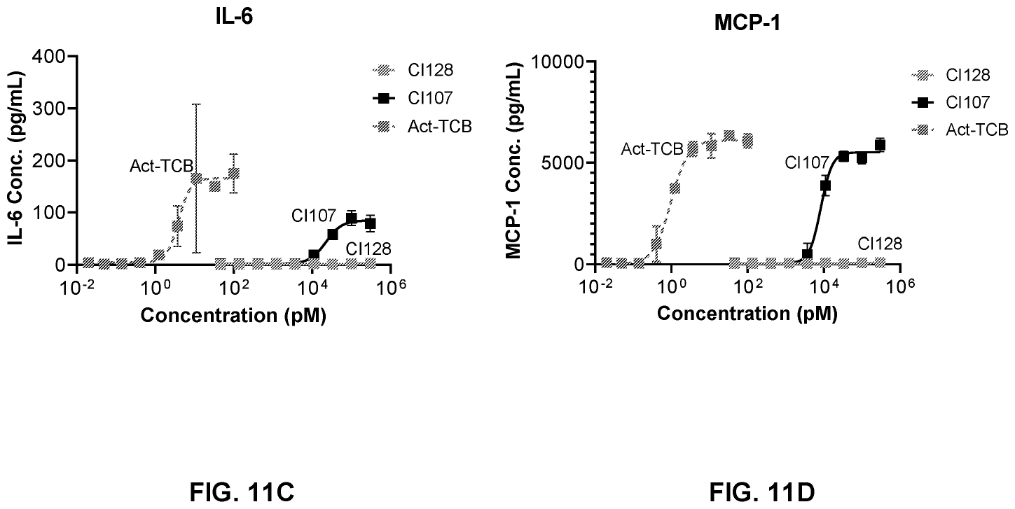

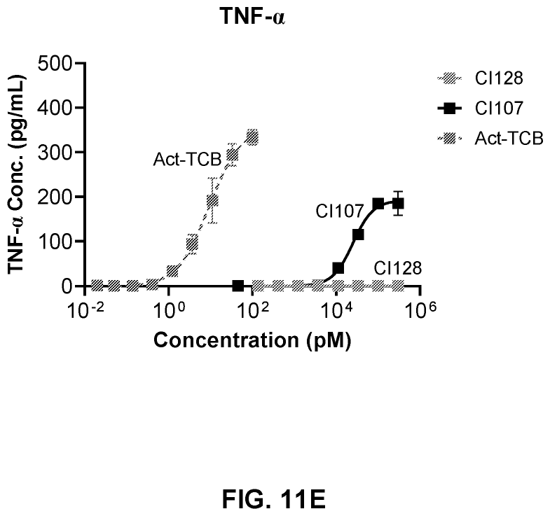

To further assess T cell activation in PBMCs co-cultured with EGFR-expressing cancer cells upon treatment with TCBs, cytokine release was evaluated after treatment with CI107, activated TCB (i.e., activated CI107), or control CI128. Levels of IFN-γ, IL-2, IL-6, MCP-1, and TNF-α were measured 16 hours after treatment with increasing concentrations of TCB. As shown in FIGS. 11A-11E, treatment with CI107 at concentrations in the 104 pM range resulted in release of each of the cytokines measured. In contrast, activated TCB resulted in cytokine release upon treatment with concentrations in the 1-100 pM range. These results were generally consistent between different PBMC donor cells and cancer cell lines (HCT116-Luc2 vs. HT29-Luc2).

Together, these data demonstrate that dual masking of the EGFR and CD3 binding domains in CI107 attenuates T cell activation in the absence of protease activation.

TCB Sensitivity to Protease Cleavage Correlates with In Vivo Anti-Tumor Efficacy and Intratumoral T Cells.

The anti-tumor efficacy of TCBs was evaluated in vivo. Immunocompromised mice harboring HT29-Luc2 tumors and engrafted with human PBMCs were treated once weekly for 3 weeks with vehicle (PBS) or 0.3 mg/kg of TCBs containing linkers with different protease sensitivities (CI011, CI040), a non-cleavable linker (CI020), or the unmasked bispecific therapeutic CI048. CI020 is expected to have minimal anti-tumor activity due to the non-cleavable linker, whereas unmasked CI048 is expected to have maximal efficacy. CI011 and CI040, which both contain EGFR and CD3 masks, have differing protease sensitivities due to different linker peptides; the protease sensitivity of CI040 is greater than that of CI011.

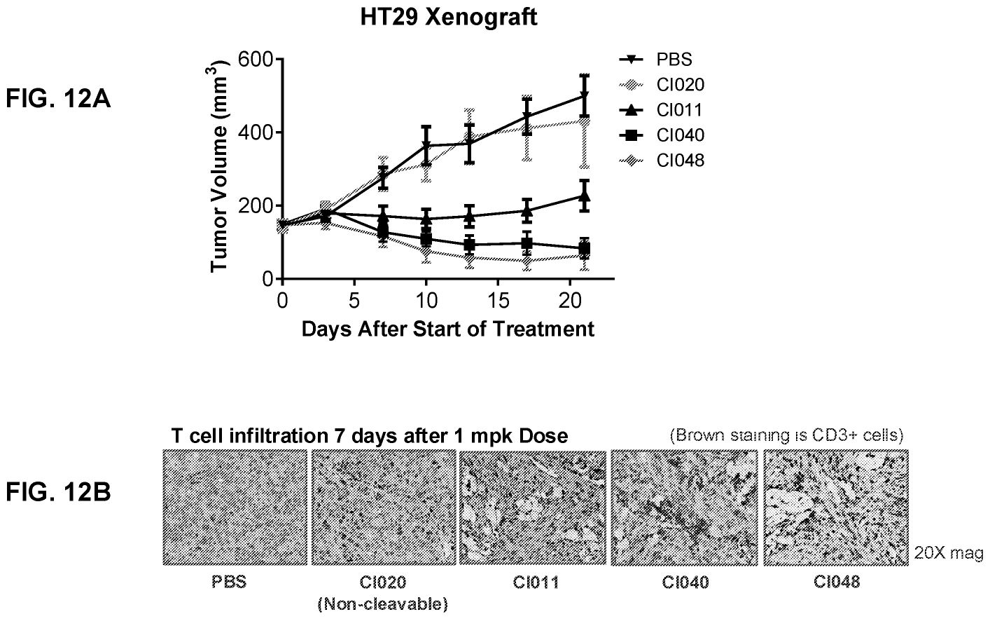

As shown in FIG. 12A, treatment with the unmasked TCB CI048 led to tumor regressions within one week after the start of treatment. Similarly, treatment with masked CI011 and CI040 also resulted in tumor regression or statis; the regression seen with CI040 correlates with the greater cleavability of the linkers in this molecule compared with CI011. In contrast, treatment with CI020, which contains non-cleavable linkers, did not affect tumor growth, indicating that protease cleavability is required for anti-tumor activity of the TCB in vivo.

To determine whether the anti-tumor efficacy mediated by the TCBs tested correlates with T cell presence in the tumors, tumors were harvested one week after animals received a 1 mg/kg dose of masked TCB or activated TCB, and immunohistochemistry for CD3 was performed. As shown in FIG. 12B, minimal numbers of T cells were observed in tumor tissue after treatment with vehicle or the non-cleavable CI020. In contrast, increased numbers of T cells were observed upon treatment with the TCB CI040 or the in vitro protease-activated TCB CI048. Again, the TCB with greater protease sensitivity (CI040) resulted in greater numbers of T cells in the tumor.

Together, these data suggest that TCBs can result in intratumoral T cells and anti-tumor efficacy in vivo that correlates with sensitivity to protease cleavage of the EGFR and CD3 binding domain masks.

Treatment with CI107 Induces Dose-Dependent Regressions of Established Xenograft Tumors.

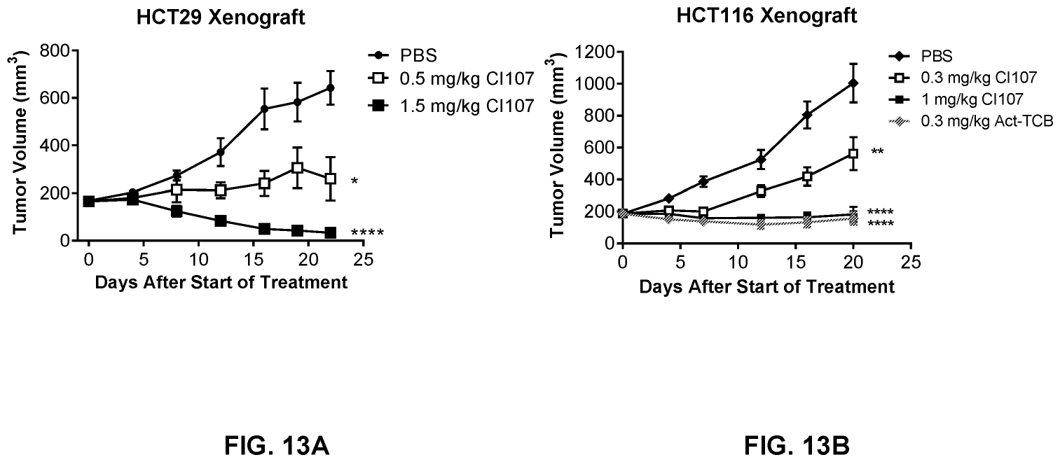

The effects of CI107 on in vivo tumor growth were evaluated. NSG mice were subcutaneously implanted with HT29 cells followed by IP injection of PBMCs, and PBMCs were allowed to engraft for approximately 11 days. Animals were then treated with vehicle, 0.5 mg/kg CI107, or 1.5 mg/kg CI107 once weekly for 3 weeks. As shown in FIG. 13A, treatment with 0.5 mg/kg CI107 resulted in tumor stasis and 1.5 mg/kg CI107 led to tumor regression starting approximately one week after treatment initiation.

The in vivo efficacy of CI107 was also evaluated in HCT116 tumors. After tumor and PBMC engraftment, animals were treated with vehicle, 0.3 mg/kg CI107, 1 mg/kg CI107, or 0.3 mg/kg activated TCB. As shown in FIG. 13B, 0.3 mg/kg CI107 delayed HCT116 tumor growth, whereas 1 mg/kg CI107 and 0.3 mg activated TCB resulted in similar levels of tumor regression and stasis for the duration of treatment.

These data demonstrate that CI107 induces dose-dependent inhibition of tumor growth and regression in HT29 and HCT116 xenograft tumors and that the anti-tumor activity of a 3-fold higher dose of CI107 is similar to that of activated TCB.

### Masked CI107 Provides Increased Safety Relative to Activated CI107 in Cynomolgus Monkeys.

The preclinical tolerability of CI107 was evaluated in cynomolgus monkey studies. Animals received a single administration of 0.06 mg/kg or 0.18 mg/kg activated CI107 (i.e., act-TCB) and 0.6 mg/kg, 2.0 mg/kg, 4.0 mg/kg, or 6.0 mg/kg CI107, and animals were followed for clinical observations. Animals treated with 0.18 mg/kg activated TCB experienced severe clinical effects, including emesis, inappetence, pale appearance, hunched posture, and thin appearance, with adverse effects noted as early as 2 hours and up to 10 days post-dose. Animals treated with 0.06 mg/kg activated TCB experienced moderate and transient clinical effects, including emesis and hunched posture on Day 1 post-dose; based on the rapid resolution of these effects, 0.06 mg/kg was defined as the maximum tolerated dose (MTD) for activated TCB. In contrast, animals treated with 2.0 mg/kg CI107 experienced only transient and mild clinical effects (emesis on Day 2), and animals treated with 0.6 mg/kg CI107 did not experience any adverse effects. Animals treated with 4.0 mg/kg CI107 experienced moderate clinical effects (including emesis at 4, 8, and 24 hours postdose and inappetence on Day 2). The animal treated with 6.0 mg/kg CI107 was found dead on Day 2. Clinical signs noted prior to death included hunched posture, pale appearance, emesis, and liquid feces post dose. Therefore, 4.0 mg/kg was considered the MTD for CI107. Overall, masked CI107 achieved a greater than 60-fold improvement in tolerability compared with activated TCB.

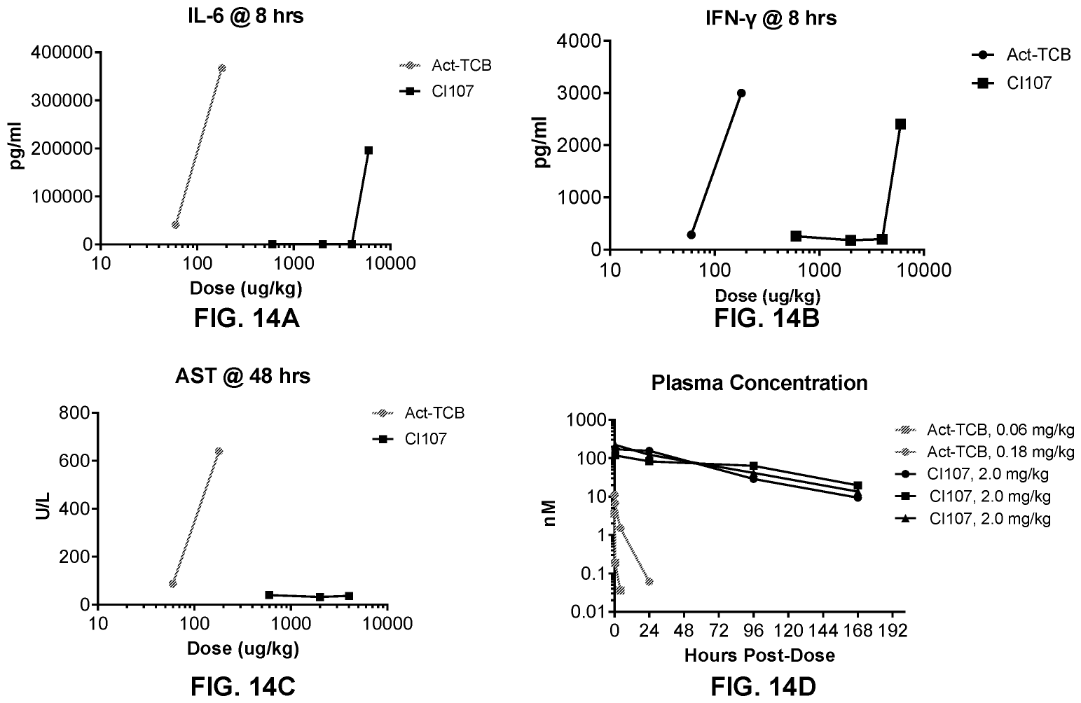

Cytokine levels were also examined after treatment with activated CI107 or masked CI107. As shown in FIG. 14, levels of IL-6 (14A) and IFN-γ (14B) were elevated in animals treated with activated TCB at 8 hours after dosing. In contrast, minimal changes in IL-6 or IFN-γ were observed after treatment with 0.6 mg/kg or 2.0 mg/kg CI107; elevated levels of these cytokines were seen only after treatment with 4.0 mg/kg CI107. Consistent with the clinical observations, CI107 shifts the cytokine release dose-response by more than 60-fold.

Analysis of serum chemistry also demonstrated differences between activated TCB and CI107. As shown in FIG. 14C, treatment with activated TCB led to dose-dependent increases in aspartate aminotransferase (AST), a marker of hepatocellular injury, at 48 hours post-dose. In contrast, no changes in AST were observed after treatment with CI107 at any of the tolerated dose levels, demonstrating improved tolerability with this masked TCB.

To address whether masking of the EGFR and CD3 binding domains affects the pharmacokinetics, the plasma concentrations of activated TCB (i.e., activated CI107) and masked CI107 after dosing were measured. As shown in FIG. 14D, activated TCB was rapidly cleared from circulation within 24 hours after dosing. In contrast, CI107 was maintained in the plasma for up to 7 days after dosing, suggesting that masking may increase exposure relative to the activated TCB. AUC (0-7) following single administration of activated TCB at 0.06 mg/kg was 0.04 day*nM (n=1), while AUC (0-7) following administration of CI107 at 2 mg/kg was 331.7 day*nM (average of n=3), demonstrating a greater than 8,000-fold increase in tolerated exposure.

This demonstrates that improvements in tolerability and pharmacokinetics observed with masked CI107 are consistent with the expected attenuation of binding to EGFR and CD3 in the normal tissue environment.

TABLE 9
Table of Sequences

| SEQ | | |
|---|---|---|
| ID | | |
| NO: | DESCRIPTION | SEQUENCE |
| 1 | Complex-66 First | CAAGGACAATCTGGCTCTGTGTCCACCACCTGTTGGTGGGACCCT |
| | Polypeptide | CCATGCACACCTAATACCGGCAGCTCTGGTGGCTCTGGCGGAAG |
| | | CGGAGGACTGTCTGGCAGATCCGATGATCACGGCGGAGGATCTG |
| | | AGGTGCAGCTGGTTGAATCTGGTGGCGGACTGGTTCAGCCTGGC |
| | | GGATCTCTGAAACTGAGCTGTGCCGCCAGCGGCTTCACCTTCAAC |
| | | AAATACGCCATGAACTGGGTCCGACAGGCCCCTGGCAAAGGCCTT |
| | | GAATGGGTCGCCAGAATCAGAAGCAAGTACAACAACTATGCCACC |
| | | TACTACGCCGACAGCGTGAAGGACAGATTCACCATCAGCCGGGA |
| | | CGACAGCAAGAACACCGCCTACCTGCAGATGAACAACCTGAAAAC |
| | | CGAGGACACCGCCGTGTACTACTGTGTGCGGCACGGCAACTTCG |
| | | GCAACAGCTACATCAGCTACTGGGCCTATTGGGGCCAGGGCACA |
| | | CTGGTCACAGTTTCTAGTGGCGGAGGCGGATCTGGCGGCGGTGG |
| | | AAGTGGCGGCGGAGGTTCTCAAACAGTGGTCACCCAAGAGCCTA |
| | | GCCTGACCGTTTCTCCTGGCGGAACCGTGACACTGACATGCGGAT |
| | | CTTCTACAGGCGCCGTGACCAGCGGCAACTACCCTAATTGGGTGC |
| | | AGCAGAAGCCAGGCCAGGCTCCTAGAGGACTGATCGGCGGCACA |
| | | AAGTTTCTGGCTCCCGGAACACCAGCCAGATTCAGCGGTTCTCTG |
| | | CTCGGAGGAAAGGCCGCTCTGACACTTTCTGGCGTGCAGCCTGA |
| | | GGATGAGGCCGAGTACTATTGCGTGCTGTGGTACAGCAACAGATG |
| | | GGTGTTCGGCGGAGGCACCAAGCTGACAGTTCTTGGAGGTGGCG |
| | | GTAGCCAGGTCCAGCTGAAACAATCTGGACCCGGACTCGTGCAG |
| | | CCAAGCCAGAGCCTGTCTATCACCTGTACCGTGTCCGGCTTCAGC |
| | | CTGACCAATTACGGCGTGCACTGGGTTCGACAATCTCCCGGCAAG |
| | | GGACTCGAATGGCTGGGAGTGATTTGGAGCGGCGGCAACACCGA |
| | | CTACAACACCCCATTCACCAGCAGACTGAGCATCAACAAGGACAA |
| | | CAGCAAGTCCCAGGTGTTCTTCAAGATGAACTCCCTGCAGAGCCA |
| | | GGATACCGCCATCTATTACTGCGCTCGGGCCCTGACCTACTATGA |
| | | CTACGAGTTTGCCTACTGGGGACAGGGAACCCTCGTGACAGTGTC |
| | | TGCTGCTAGCACAAAGGGCCCTAGCGTTTTCCCACTGGCTCCCAG |
| | | CAGCAAGTCTACATCCGGTGGAACAGCCGCTCTGGGCTGCCTGG |
| | | TCAAGGATTACTTTCCCGAGCCAGTGACCGTGTCCTGGAATAGCG |
| | | GAGCACTGACATCTGGCGTGCACACATTTCCAGCCGTGCTGCAGT |
| | | CTAGCGGCCTGTACTCTCTGTCCAGCGTTGTGACAGTGCCCAGCA |
| | | GCTCTCTGGGCACCCAGACCTACATCTGCAATGTGAACCACAAGC |
| | | CTAGCAACACCAAGGTGGACAAGAAGGTGGAACCCAAGAGCTGC |
| | | GATAAGACACACACCTGTCCTCCATGTCCTGCTCCAGAGCTGCTC |
| | | GGAGGCCCTTCCGTGTTTCTGTTCCCTCCAAAGCCTAAGGACACC |
| | | CTGATGATCAGCAGAACCCCTGAAGTGACCTGCGTGGTGGTGGAT |
| | | GTGTCCCACGAGGATCCCGAAGTGAAGTTCAATTGGTACGTCGAC |
| | | GGCGTGGAAGTGCACAATGCCAAGACCAAGCCTTGCGAGGAACA |
| | | GTACGGCAGCACCTACAGATGCGTGTCCGTGCTGACAGTGCTGC |
| | | ACCAGGATTGGCTGAACGGCAAAGAGTACAAGTGCAAGGTGTCCA |
| | | ACAAGGCCCTGCCTGCTCCTATCGAGAAAACCATCAGCAAGGCCA |
| | | AGGGCCAGCCTAGAGAACCCCAGGTGTACACACTGCCTCCAAGC |
| | | CGGAAAGAGATGACCAAGAATCAGGTGTCCCTGACCTGCCTGGTC |
| | | AAGGGCTTCTACCCTTCCGATATCGCCGTGGAATGGGAGAGCAAT |
| | | GGACAGCCCGAGAACAACTACAAGACAACCCCTCCTGTGCTGAAG |
| | | TCCGACGGCTCATTCTTCCTGTACAGCAAGCTGACCGTGGACAAG |
| | | AGCAGATGGCAGCAGGGCAACGTGTTCAGCTGCAGCGTGATGCA |
| | | CGAGGCCCTGCACAACCACTACACCCAGAAGTCCCTGTCTCTGAG |
| | | CCCCGGCAAA |
| 2 | Complex-66 First | QGQSGSVSTTCWWDPPCTPNTGSSGGSGGSGGLSGRSDDHGGGS |
| | Polypeptide | EVQLVESGGGLVQPGGSLKLSCAASGFTFNKYAMNWVRQAPGKGL |
| | | EWVARIRSKYNNYATYYADSVKDRFTISRDDSKNTAYLQMNNLKTED |
| | | TAVYYCVRHGNFGNSYISYWAYWGQGTLVTVSSGGGGSGGGGSG |
| | | GGGSQTVVTQEPSLTVSPGGTVTLTCGSSTGAVTSGNYPNWVQQK |
| | | PGQAPRGLIGGTKFLAPGTPARFSGSLLGGKAALTLSGVQPEDEAEY |
| | | YCVLWYSNRWVFGGGTKLTVLGGGGSQVQLKQSGPGLVQPSQSLSI |
| | | TCTVSGFSLTNYGVHWVRQSPGKGLEWLGVIWSGGNTDYNTPFTSR |
| | | LSINKDNSKSQVFFKMNSLQSQDTAIYYCARALTYYDYEFAYWGQGT |
| | | LVTVSAASTKGPSVFPLAPSSKSTSGGTAALGCLVKDYFPEPVTVSW |
| | | NSGALTSGVHTFPAVLQSSGLYSLSSVVTVPSSSLGTQTYICNVNHKP |
| | | SNTKVDKKVEPKSCDKTHTCPPCPAPEFEGGPSVFLFPPKPKDTLMI |
| | | SRTPEVTCVVVDVSHEDPEVKFNWYVDGVEVHNAKTKPREEQYQST |
| | | YRVVSVLTVLHQDWLNGKEYKCKVSNKALPASIEKTISKAKGQPREP |
| | | QVYTLPPSREEMTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| 3 | Spacer | QGQSGS |
| 4 | Complex-66 Anti- | VSTTCWWDPPCTPNT |
| | CD3 MM ML 15 | |
| 5 | CM 0011 | LSGRSDDH |
| 6 | Anti-CD3 scFv | EVQLVESGGGLVQPGGSLKLSCAASGFTFNKYAMNWVRQAPGKGL |
| | | EWVARIRSKYNNYATYYADSVKDRFTISRDDSKNTAYLQMNNLKTED |
| | | TAVYYCVRHGNFGNSYISYWAYWGQGTLVTVSSGGGGSGGGGSG |
| | | GGGSQTVVTQEPSLTVSPGGTVTLTCGSSTGAVTSGNYPNWVQQK |
| | | PGQAPRGLIGGTKFLAPGTPARFSGSLLGGKAALTLSGVQPEDEAEY |
| | | YCVLWYSNRWVFGGGTKLTVL |
| 7 | Complex-66/CI106 | QVQLKQSGPGLVQPSQSLSITCTVSGFSLTNYGVHWVRQSPGKGLE |
| | Anti-EGFR VH | WLGVIWSGGNTDYNTPFTSRLSINKDNSKSQVFFKMNSLQSQDTAIY |
| | | YCARALTYYDYEFAYWGQGTLVTVSA |
| 8 | CH1-Human IgG1- | ASTKGPSVFPLAPSSKSTSGGTAALGCLVKDYFPEPVTVSWNSGALT |
| | Complex-66 | SGVHTFPAVLQSSGLYSLSSVVTVPSSSLGTQTYICNVNHKPSNTKV |
| | | DKKV |
| 9 | CH1-Human lgG4 | ASTKGPSVFPLAPCSRSTSESTAALGCLVKDYFPEPVTVSWNSGALT |
| | | SGVHTFPAVLQSSGLYSLSSVVTVPSSSLGTKTYTCNVDHKPSNTKV |
| | | DKRV |
| 10 | Hinge-A, | EPKSCDKTHTCPPC |
| | Complex-66/CI106 | |
| 11 | Hinge-B | DKTHTCPPC |
| | Complex-166 | |
| 12 | Blank | |
| 13 | Blank | |
| 14 | Blank | |
| 15 | Complex-66/CI106 | CAAGGACAATCTGGACAGGGCCTGAGCTGTGAAGGCTGGGCCAT |
| | Light Chain | GAATAGAGAGCAGTGCAGAGCTGGCGGCGGATCTTCTGGCGGCT |
| | | CTATCTCTTCTGGACTGCTGAGCGGCAGAAGCGATCAACACGGCG |
| | | GAGGCTCTCAGATCCTGCTGACACAGAGCCCCGTGATCCTGTCTG |
| | | TGTCTCCTGGCGAGAGAGTGTCCTTCAGCTGTAGAGCCAGCCAGT |
| | | CCATCGGCACCAACATCCACTGGTATCAGCAGCGGACCAACGGC |
| | | AGCCCCAGACTGCTGATTAAGTACGCCAGCGAGAGCATCAGCGG |
| | | CATCCCCAGCAGATTTTCTGGCAGCGGCTCTGGCACCGACTTCAC |
| | | CCTGAGCATCAACAGCGTGGAAAGCGAGGATATCGCCGACTACTA |
| | | CTGCCAGCAGAACAACAACTGGCCCACCACCTTTGGAGCCGGCA |
| | | CCAAGCTGGAACTGAAGAGAACAGTGGCCGCTCCTAGCGTGTTCA |
| | | TCTTCCCACCTTCCGACGAGCAGCTGAAAAGCGGCACAGCCTCTG |
| | | TCGTGTGCCTGCTGAACAACTTCTACCCCAGAGAAGCCAAGGTGC |
| | | AGTGGAAGGTGGACAACGCCCTGCAGAGCGGCAATAGCCAAGAG |
| | | TCTGTGACCGAGCAGGACAGCAAGGACTCCACCTACAGCCTGAG |
| | | CAGCACCCTGACACTGAGCAAGGCCGACTACGAGAAGCACAAAG |
| | | TGTACGCCTGCGAAGTGACCCACCAGGGCCTTTCTAGCCCTGTGA |
| | | CCAAGAGCTTCAACCGGGGCGAGTGT |
| 16 | Complex66/CI106 | QGQSGQGLSCEGWAMNREQCRAGGGSSGGSISSGLLSGRSDQHG |
| | Light Chain | GGSQILLTQSPVILSVSPGERVSFSCRASQSIGTNIHWYQQRTNGSPR |
| | | LLIKYASESISGIPSRFSGSGSGTDFTLSINSVESEDIADYYCQQNNNW |
| | | PTTFGAGTKLELKRTVAAPSVFIFPPSDEQLKSGTASVVCLLNNFYPR |
| | | EAKVQWKVDNALQSGNSQESVTEQDSKDSTYSLSSTLTLSKADYEK |
| | | HKVYACEVTHQGLSSPVTKSFNRGEC |
| 17 | Spacer-Light | QGQSGQG |
| | Chain-2^{nd} | |
| | Polypeptide | |
| 18 | MM-anti-EGFR | LSCEGWAMNREQCRA |
| | COMPLEX-66 | |
| 19 | CM | ISSGLLSGRSDQH |
| 20 | VL-anti-EGFR | QILLTQSPVILSVSPGERVSFSCRASQSIGTNIHWYQQRTNGSPRLLIK |
| | COMPLEX-66/106 | YASESISGIPSRFSGSGSGTDFTLSINSVESEDIADYYCQQNNNWPTT |
| | | FGAGTKLELK |
| 21 | CL Domain- | RTVAAPSVFIFPPSDEQLKSGTASVVCLLNNFYPREAKVQWKVDNAL |
| | COMPLEX-66/106 | QSGNSQESVTEQDSKDSTYSLSSTLTLSKADYEKHKVYACEVTHQGL |
| | Light kappa | SSPVTKSFNRGEC |
| | Constant Domain | |
| 22 | CI106-1^{st} | CAAGGACAATCTGGATCCGGCTATCTGTGGGGCTGCGAGTGGAAT |
| | Polypeptide | TGTGGCGGCATCACAACAGGCTCTAGCGGCGGAAGCGGAGGATC |
| | | TGGTGGACTGTCTGGCAGATCCGATGATCATGGCGGCGGATCCC |
| | | AGACCGTGGTCACACAAGAGCCTAGCTTCTCCGTGTCTCCTGGCG |
| | | GCACAGTGACCCTGACATGCAGATCTTCTACAGGCGCCGTGACCA |
| | | CCAGCAACTACGCCAATTGGGTGCAGCAGACCCCTGGACAGGCT |
| | | CCTAGAGGACTGATCGGCGGCACCAACAAAAGAGCCCCTGGCGT |
| | | CCCAGATAGATTCAGCGGCTCTATCCTGGGCAACAAGGCCGCACT |
| | | GACAATCACAGGCGCCCAGGCCGATGACGAGAGCGATTACTATTG |
| | | CGCCCTGTGGTACAGCAACCTGTGGGTTTTCGGCGGAGGCACCA |
| | | AGCTGACAGTTCTTGGCGGAGGCGGAAGTGGTGGTGGCGGATCT |
| | | GGTGGCGGTGGATCTGAAGTGCAGCTGGTGGAATCTGGCGGAGG |
| | | ACTTGTTCAGCCAGGCGGCTCTCTGAAGCTGTCTTGTGCCGCCTC |
| | | CGGCTTCACCTTTAGCACCTACGCCATGAACTGGGTCCGACAGGC |
| | | CTCTGGCAAAGGCCTGGAATGGGTCGGACGGATCAGAAGCAAGT |
| | | ACAACAATTACGCCACCTACTACGCCGACAGCGTGAAGGACAGAT |
| | | TCACCATCAGCCGGGACGACAGCAAGAACACCGCCTACCTGCAG |
| | | ATGAACAGCCTGAAAACCGAGGACACCGCCGTGTACTACTGCACC |
| | | AGACACGGCAACTTCGGCAACAGCTATGTGTCTTGGTTTGCCTAC |
| | | TGGGGCCAGGGCACACTGGTCACAGTTAGTTCTGGCGGCGGAGG |
| | | TTCTCAGGTGCAGCTGAAACAGTCTGGCCCTGGACTGGTGCAGCC |
| | | TAGCCAGTCTCTGAGCATCACCTGTACCGTGTCCGGCTTCTCCCT |
| | | GACCAATTACGGCGTGCACTGGGTTCGACAATCCCCAGGCAAGG |
| | | GACTCGAATGGCTGGGAGTGATTTGGAGCGGCGGCAACACCGAC |
| | | TACAACACCCCATTCACCAGCAGACTGTCCATCAACAAGGACAAC |
| | | AGCAAGTCCCAGGTGTTCTTCAAGATGAACTCCCTGCAGAGCCAG |
| | | GATACCGCCATCTATTACTGCGCTCGGGCCCTGACCTACTATGAC |
| | | TACGAGTTCGCCTATTGGGGACAGGGAACCCTCGTGACAGTGTCT |
| | | GCCGCTAGCACAAAGGGCCCTAGCGTTTTCCCACTGGCTCCCAG |
| | | CAGCAAGTCTACATCCGGTGGAACAGCCGCTCTGGGCTGCCTGG |
| | | TCAAGGATTACTTTCCCGAGCCAGTGACCGTGTCCTGGAATAGCG |
| | | GAGCACTGACATCTGGCGTGCACACATTTCCAGCCGTGCTGCAGT |
| | | CTAGCGGCCTGTACTCTCTGTCCAGCGTTGTGACAGTGCCCAGCA |
| | | GCTCTCTGGGCACCCAGACCTACATCTGCAATGTGAACCACAAGC |
| | | CTAGCAACACCAAGGTGGACAAGAAGGTGGAACCCAAGAGCTGC |
| | | GATAAGACACACACCTGTCCTCCATGTCCTGCTCCAGAGCTGCTC |
| | | GGAGGCCCTTCCGTGTTTCTGTTCCCTCCAAAGCCTAAGGACACC |
| | | CTGATGATCAGCAGAACCCCTGAAGTGACCTGCGTGGTGGTGGAT |
| | | GTGTCCCACGAGGATCCCGAAGTGAAGTTCAATTGGTACGTCGAC |
| | | GGCGTGGAAGTGCACAATGCCAAGACCAAGCCTTGCGAGGAACA |
| | | GTACGGCAGCACCTACAGATGCGTGTCCGTGCTGACAGTGCTGC |
| | | ACCAGGATTGGCTGAACGGCAAAGAGTACAAGTGCAAGGTGTCCA |
| | | ACAAGGCCCTGCCTGCTCCTATCGAGAAAACCATCAGCAAGGCCA |
| | | AGGGCCAGCCTAGAGAACCCCAGGTGTACACACTGCCTCCAAGC |
| | | CGGAAAGAGATGACCAAGAATCAGGTGTCCCTGACCTGCCTGGTC |
| | | AAGGGCTTCTACCCTTCCGATATCGCCGTGGAATGGGAGAGCAAT |
| | | GGACAGCCCGAGAACAACTACAAGACAACCCCTCCTGTGCTGAAG |
| | | TCCGACGGCTCATTCTTCCTGTACAGCAAGCTGACCGTGGACAAG |
| | | AGCAGATGGCAGCAGGGCAACGTGTTCAGCTGCAGCGTGATGCA |
| | | CGAGGCCCTGCACAACCACTACACCCAGAAGTCCCTGTCTCTGAG |
| | | CCCCGGCAAA |
| 23 | CI106-1st | QGQSGSGYLWGCEWNCGGITTGSSGGSGGSGGLSGRSDDHGGGS |
| | Polypeptide | QTVVTQEPSFSVSPGGTVTLTCRSSTGAVTTSNYANWVQQTPGQAP |
| | | RGLIGGTNKRAPGVPDRFSGSILGNKAALTITGAQADDESDYYCALW |
| | | YSNLWVFGGGTKLTVLGGGGSGGGGSGGGGSEVQLVESGGGLVQ |
| | | PGGSLKLSCAASGFTFSTYAMNWVRQASGKGLEWVGRIRSKYNNYA |
| | | TYYADSVKDRFTISRDDSKNTAYLQMNSLKTEDTAVYYCTRHGNFGN |
| | | SYVSWFAYWGQGTLVTVSSGGGGSQVQLKQSGPGLVQPSQSLSIT |
| | | CTVSGFSLTNYGVHWVRQSPGKGLEWLGVIWSGGNTDYNTPFTSRL |
| | | SINKDNSKSQVFFKMNSLQSQDTAIYYCARALTYYDYEFAYWGQGTL |
| | | VTVSAASTKGPSVFPLAPSSKSTSGGTAALGCLVKDYFPEPVTVSWN |
| | | SGALTSGVHTFPAVLQSSGLYSLSSVVTVPSSSLGTQTYICNVNHKPS |
| | | NTKVDKKVEPKSCDKTHTCPPCPAPELLGGPSVFLFPPKPKDTLMISR |
| | | TPEVTCVVVDVSHEDPEVKFNWYVDGVEVHNAKTKPCEEQYGSTYR |
| | | CVSVLTVLHQDWLNGKEYKCKVSNKALPAPIEKTISKAKGQPREPQV |
| | | YTLPPSRKEMTKNQVSLTCLVKGFYPSDIAVEWESNGQPENNYKTTP |
| | | PVLKSDGSFFLYSKLTVDKSRWQQGNVFSCSVMHEALHNHYTQKSL |
| | | SLSPGK |
| 24 | CI106-Anti-CD3 | GYLWGCEWNCGGITT |
| | MM | |
| 25 | CI106-Anti-CD3 | QTVVTQEPSFSVSPGGTVTLTCRSSTGAVTTSNYANWVQQTPGQAP |
| | scFv | RGLIGGTNKRAPGVPDRFSGSILGNKAALTITGAQADDESDYYCALW |
| | | YSNLWVFGGGTKLTVLGGGGSGGGGSGGGGSEVQLVESGGGLVQ |
| | | PGGSLKLSCAASGFTFSTYAMNWVRQASGKGLEWVGRIRSKYNNYA |
| | | TYYADSVKDRFTISRDDSKNTAYLQMNSLKTEDTAVYYCTRHGNFGN |
| | | SYVSWFAYWGQGTLVTVSS |
| 26 | CI106 Heavy | CAAGGCCAGTCTGGATCCGGTTATCTGTGGGGTTGCGAGTGGAAT |
| | Chain | TGCGGAGGGATCACTACAGGCTCGAGCGGTGGCAGCGGTGGCTC |
| | | TGGTGGTCTGAGCGGCCGTTCCGATGATCATGGCGGCGGTTCTC |
| | | AAACTGTAGTAACTCAAGAACCAAGCTTCTCCGTCTCCCCTGGGG |
| | | GAACAGTCACACTTACCTGCCGAAGTAGTACAGGTGCTGTTACGA |
| | | CCAGTAACTATGCCAATTGGGTACAACAAACGCCTGGTCAGGCTC |
| | | CGCGCGGATTGATAGGAGGCACGAATAAACGGGCACCCGGTGTC |
| | | CCGGACAGATTCAGCGGAAGCATACTCGGTAATAAGGCAGCTCTT |
| | | ACTATCACTGGGGCCCAAGCTGATGATGAAAGTGATTATTATTGTG |
| | | CGCTCTGGTACAGCAACCTCTGGGTGTTTGGGGGTGGCACGAAA |
| | | CTTACTGTCTTGGGCGGCGGCGGATCAGGGGGAGGTGGCTCTGG |
| | | AGGAGGAGGCTCAGAAGTCCAACTGGTCGAATCCGGGGGAGGGC |
| | | TCGTACAGCCGGGTGGGTCCCTCAAACTCTCTTGTGCGGCCTCAG |
| | | GGTTTACCTTCAGTACATACGCGATGAATTGGGTCCGGCAGGCCA |
| | | GTGGGAAAGGGCTCGAATGGGTAGGACGAATCCGATCAAAATACA |
| | | ACAACTACGCTACTTATTACGCTGATTCCGTGAAGGACAGATTCAC |
| | | AATATCCCGCGACGATAGCAAGAATACGGCATATCTTCAGATGAAT |
| | | TCTCTTAAAACTGAGGATACCGCTGTGTATTACTGCACAAGACATG |
| | | GTAATTTTGGAAACTCATATGTCTCTTGGTTCGCTTATTGGGGACA |
| | | GGGCACGTTGGTTACCGTGTCTAGCGGAGGTGGTGGATCCCAGG |
| | | TGCAGCTGAAACAGAGCGGCCCGGGCCTGGTGCAGCCGAGCCA |
| | | GAGCCTGAGCATTACCTGCACCGTGAGCGGCTTTAGCCTGACCAA |
| | | CTATGGCGTGCATTGGGTGCGCCAGAGCCCGGGCAAAGGCCTGG |
| | | AATGGCTGGGCGTGATTTGGAGCGGCGGCAACACCGATTATAACA |
| | | CCCCGTTTACCAGCCGCCTGAGCATTAACAAAGATAACAGCAAAA |
| | | GCCAGGTGTTTTTTAAAATGAACAGCCTGCAAAGCCAGGATACCG |
| | | CGATTTATTATTGCGCGCGCGCGCTGACCTATTATGATTATGAATT |
| | | TGCGTATTGGGGCCAGGGCACCCTGGTGACCGTGAGCGCGGCTA |
| | | GCACCAAGGGCCCATCGGTCTTCCCCCTGGCACCCTCCTCCAAG |
| | | AGCACCTCTGGGGGCACAGCGGCCCTGGGCTGCCTGGTCAAGGA |
| | | CTACTTCCCCGAACCGGTGACGGTGTCGTGGAACTCAGGCGCCC |
| | | TGACCAGCGGCGTGCACACCTTCCCGGCTGTCCTACAGTCCTCAG |
| | | GACTCTACTCCCTCAGCAGCGTGGTGACCGTGCCCTCCAGCAGCT |
| | | TGGGCACCCAGACCTACATCTGCAACGTGAATCACAAGCCCAGCA |
| | | ACACCAAGGTGGACAAGAAAGTTGAGCCCAAATCTTGTGACAAAA |
| | | CTCACACATGCCCACCGTGCCCAGCACCTGAATTTGAAGGGGGAC |
| | | CGTCAGTCTTCCTCTTCCCCCCAAAACCCAAGGACACCCTCATGA |
| | | TCTCCCGGACCCCTGAGGTCACATGCGTGGTGGTGGACGTGAGC |
| | | CACGAAGACCCTGAGGTCAAGTTCAACTGGTACGTGGACGGCGT |
| | | GGAGGTGCATAATGCCAAGACAAAGCCGCGGGAGGAGCAGTACC |
| | | AGAGCACGTACCGTGTGGTCAGCGTCCTCACCGTCCTGCACCAG |
| | | GACTGGCTGAATGGCAAGGAGTACAAGTGCAAGGTCTCCAACAAA |
| | | GCCCTCCCAGCCTCAATCGAGAAAACCATCTCCAAAGCCAAAGGG |
| | | CAGCCCCGAGAACCACAGGTGTACACCCTGCCCCCATCCCGGGA |
| | | GGAGATGACCAAGAACCAGGTCAGCCTGACCTGCCTGGTCAAAG |
| | | GCTTCTATCCCAGCGACATCGCCGTGGAGTGGGAGAGCAATGGG |
| | | CAGCCGGAGAACAACTACAAGACCACGCCTCCCGTGCTGGACTC |
| | | CGACGGCTCCTTCTTCCTCTACAGCAAGCTCACCGTGGACAAGAG |
| | | CAGGTGGCAGCAGGGGAACGTCTTCTCATGCTCCGTGATGCATGA |
| | | GGCTCTGCACAACCACTACACGCAGAAGAGCCTCTCCCTGTCTCC |
| | | GGGTAAA |
| 27 | CI106-Heavy | QGQSGSGYLWGCEWNCGGITTGSSGGSGGSGGLSGRSDDHGGGS |
| | Chain | QTVVTQEPSFSVSPGGTVTLTCRSSTGAVTTSNYANWVQQTPGQAP |
| | | RGLIGGTNKRAPGVPDRFSGSILGNKAALTITGAQADDESDYYCALW |
| | | YSNLWVFGGGTKLTVLGGGGSGGGGSGGGGSEVQLVESGGGLVQ |
| | | PGGSLKLSCAASGFTFSTYAMNWVRQASGKGLEWVGRIRSKYNNYA |
| | | TYYADSVKDRFTISRDDSKNTAYLQMNSLKTEDTAVYYCTRHGNFGN |
| | | SYVSWFAYWGQGTLVTVSSGGGGSQVQLKQSGPGLVQPSQSLSIT |
| | | CTVSGFSLTNYGVHWVRQSPGKGLEWLGVIWSGGNTDYNTPFTSRL |
| | | SINKDNSKSQVFFKMNSLQSQDTAIYYCARALTYYDYEFAYWGQGTL |
| | | VTVSAASTKGPSVFPLAPSSKSTSGGTAALGCLVKDYFPEPVTVSWN |
| | | SGALTSGVHTFPAVLQSSGLYSLSSVVTVPSSSLGTQTYICNVNHKPS |
| | | NTKVDKKVEPKSCDKTHTCPPCPAPEFEGGPSVFLFPPKPKDTLMIS |
| | | RTPEVTCVVVDVSHEDPEVKFNWYVDGVEVHNAKTKPREEQYQSTY |
| | | RVVSVLTVLHQDWLNGKEYKCKVSNKALPASIEKTISKAKGQPREPQ |
| | | VYTLPPSREEMTKNQVSLTCLVKGFYPSDIAVEWESNGQPENNYKTT |
| | | PPVLDSDGSFFLYSKLTVDKSRWQQGNVFSCSVMHEALHNHYTQKS |
| | | LSLSPGK |
| 28 | Fc | PAPEFEGGPSVFLFPPKPKDTLMISRTPEVTCVVVDVSHEDPEVKFN |
| | without C-terminal | WYVDGVEVHNAKTKPREEQYQSTYRVVSVLTVLHQDWLNGKEYKC |
| | lysine | KVSNKALPASIEKTISKAKGQPREPQVYTLPPSREEMTKNQVSLTCLV |
| | | KGFYPSDIAVEWESNGQPENNYKTTPPVLDSDGSFFLYSKLTVDKSR |
| | | WQQGNVFSCSVMHEALHNHYTQKSLSLSPG |
| 29 | CI106-CH1- | ASTKGPSVFPLAPSSKSTSGGTAALGCLVKDYFPEPVTVSWNSGALT |
| | Hinge-Fc | SGVHTFPAVLQSSGLYSLSSVVTVPSSSLGTQTYICNVNHKPSNTKV |
| | | DKKVEPKSCDKTHTCPPCPAPEFEGGPSVFLFPPKPKDTLMISRTPE |
| | | VTCVVVDVSHEDPEVKFNWYVDGVEVHNAKTKPREEQYQSTYRVVS |
| | | VLTVLHQDWLNGKEYKCKVSNKALPASIEKTISKAKGQPREPQVYTLP |
| | | PSREEMTKNQVSLTCLVKGFYPSDIAVEWESNGQPENNYKTTPPVLD |
| | | SDGSFFLYSKLTVDKSRWQQGNVFSCSVMHEALHNHYTQKSLSLSP |
| | | GK |
| 30 | 12C Anti-CD3 VL1 | QTVVTQEPSLTVSPGGTVTLTCGSSTGAVTSGNYPNWVQQKPGQAP |
| | | RGLIGGTKFLAPGTPARFSGSLLGGKAALTLSGVQPEDEAEYYCVLW |
| | | YSNRWVFGGGTKLTV |
| 31 | 12C Anti-CD3 VH1 | EVQLVESGGGLVQPGGSLKLSCAASGFTFNKYAMNWVRQAPGKGL |
| | | EWVARIRSKYNNYATYYADSVKDRFTISRDDSKNTAYLQMNNLKTED |
| | | TAVYYCVRHGNFGNSYISYWAYWGQGTLVTVSS |
| 32 | v16 Anti-CD3 VL1 | QTVVTQEPSFSVSPGGTVTLTCRSSTGAVTTSNYANWVQQTPGQAP |
| | | RGLIGGTNKRAPGVPDRFSGSILGNKAALTITGAQADDESDYYCALW |
| | | YSNLWVFGGGTKLTVL |
| 33 | V16 Anti-CD3 VH1 | EVQLVESGGGLVQPGGSLKLSCAASGFTFSTYAMNWVRQASGKGLE |
| | | WVGRIRSKYNNYATYYADSVKDRFTISRDDSKNTAYLQMNSLKTEDT |
| | | AVYYCTRHGNFGNSYVSWFAYWGQGTLVTVSS |
| 34 | EGFR VL CDR1 | RASQSIGTNIH |
| 35 | EGFR VL CDR2 | YASESIS |
| 36 | EGFR VL CDR3 | QQNNNWPTT |
| 37 | EGFR VH CDR1 | NYGVH |
| 38 | EGFR VH CDR2 | VIWSGGNTDYNTPFTS |
| 39 | EGFR VH CDR3 | ALTYYDYEFAY |
| 40 | CD3 I2C VL | SSTGAVTSGNYPNG |
| | CDR1 | |
| 41 | CD3 I2C VL | GTKFLAP |
| | CDR2 | |
| 42 | CD3 I2C VL | VLWYSNRWV |
| | CDR3 | |
| 43 | CD3 I2C VH | KYAMN |
| | CDR1 | |
| 44 | CD3 I2C VH | RIRSKYNNYATYYADSVKD |
| | CDR2 | |
| 45 | CD3 I2C VH | HGNFGNSYISYWAY |
| | CDR3 | |
| 46 | CD3 v16 VL | RSSTGAVTTSNYAN |
| | CDR1 | |
| 47 | CD3 v16 VL | GTNKRAP |
| | CDR2 | |
| 48 | CD3 v16 VL CDR3 | ALWYSNLWV |
| 49 | CD3 v16 VH | TYAMN |
| | CDR1 | |
| 50 | CD3 v16 VH | RIRSKYNNYATYYADSVKD |
| | CDR2 | |
| 51 | CD3 v16 VH | HGNFGNSYVSWFAY |
| | CDR3 | |
| 52 | CD3 MM | MMYCGGNEVLCGPRV |
| 53 | CD3 MM | GYRWGCEWNCGGITT |
| 54 | CD3 MM | MMYCGGNEIFCEPRG |
| 55 | CD3 MM | GYGWGCEWNCGGSSP |
| 56 | CD3 MM | MMYCGGNEIFCGPRG |
| 57 | CM | LSGRSDNH |
| 58 | CM | TSTSGRSANPRG |
| 59 | CM | VHMPLGFLGP |
| 60 | CM | AVGLLAPP |
| 61 | CM | QNQALRMA |
| 62 | CM | ISSGLLSS |
| 63 | CM | ISSGLLSGRSDNH |
| 64 | CM | LSGRSGNH |
| 65 | CM | LSGRSDIH |
| 66 | CM | LSGRSDQH |
| 67 | CM | LSGRSDTH |
| 68 | CM | LSGRSDYH |
| 69 | CM | LSGRSDNP |
| 70 | CM | LSGRSANP |
| 71 | CM | LSGRSANI |
| 72 | CM | LSGRSDNI |
| 73 | CM | ISSGLLSGRSANPRG |
| 74 | CM | AVGLLAPPTSGRSANPRG |
| 75 | CM | AVGLLAPPSGRSANPRG |
| 76 | CM | ISSGLLSGRSDDH |
| 77 | CM | ISSGLLSGRSDIH |
| 78 | CM | ISSGLLSGRSDTH |
| 79 | CM | ISSGLLSGRSDYH |
| 80 | CM | ISSGLLSGRSDNP |
| 81 | CM | ISSGLLSGRSANP |
| 82 | CM | ISSGLLSGRSANI |
| 83 | CM | AVGLLAPPGGLSGRSDDH |
| 84 | CM | AVGLLAPPGGLSGRSDIH |
| 85 | CM | AVGLLAPPGGLSGRSDQH |
| 86 | CM | AVGLLAPPGGLSGRSDTH |
| 87 | CM | AVGLLAPPGGLSGRSDYH |
| 88 | CM | AVGLLAPPGGLSGRSDNP |
| 89 | CM | AVGLLAPPGGLSGRSANP |
| 90 | CM | AVGLLAPPGGLSGRSANI |
| 91 | CM | ISSGLLSGRSDNI |
| 92 | CM | AVGLLAPPGGLSGRSDNI |
| 93 | CM | ISSGLLSGRSGNH |
| 94 | Linker | GGGS |
| 95 | Linker | GSGGS |
| 96 | Linker | GGSG |
| 97 | Linker | GGSGG |
| 98 | Linker | GSGSG |
| 99 | Linker | GSGGG |
| 100 | Linker | GGGSG |
| 101 | Linker | GSSSG |
| 102 | Linker | GGGGSGGGGSGGGGSGS |
| 103 | Linker | GGGGSGS |
| 104 | Linker | GGGGSGGGGSGGGGS |
| 105 | Linker | GGGGSGGGGSGGGGSGGGGS |
| 106 | Linker | GGGGS |
| 107 | Linker | GGGGSGGGGS |
| 108 | Linker | GGGSGGGS |
| 109 | Linker | GGGSGGGSGGGS |
| 110 | Linker | GSSGGSGGSGG |
| 111 | Linker | GGGSGGGGSGGGGSGGGGSGGGGS |
| 112 | Linker | GSTSGSGKPGSSEGST |
| 113 | Linker | SKYGPPCPPCPAPEFLG |
| 114 | Linker | GGSLDPKGGGGS |
| 115 | Linker | PKSCDKTHTCPPCPAPELLG |
| 116 | Linker | GKSSGSGSESKS |
| 117 | Linker | GSTSGSGKSSEGKG |
| 118 | Linker | GSTSGSGKSSEGSGSTKG |
| 119 | Linker | GSTSGSGKPGSGEGSTKG |
| 120 | Complex-66-1^{st} | VSTTCWWDPPCTPNTGSSGGSGGSGGLSGRSDDHGGGSEVQLVE |
| | polypeptide | SGGGLVQPGGSLKLSCAASGFTFNKYAMNWVRQAPGKGLEWVARI |
| | without spacer and | RSKYNNYATYYADSVKDRFTISRDDSKNTAYLQMNNLKTEDTAVYYC |
| | without C-terminal | VRHGNFGNSYISYWAYWGQGTLVTVSSGGGGSGGGGSGGGGSQT |
| | lysine | VVTQEPSLTVSPGGTVTLTCGSSTGAVTSGNYPNWVQQKPGQAPR |
| | | GLIGGTKFLAPGTPARFSGSLLGGKAALTLSGVQPEDEAEYYCVLWY |
| | | SNRWVFGGGTKLTVLGGGGSQVQLKQSGPGLVQPSQSLSITCTVSG |
| | | FSLTNYGVHWVRQSPGKGLEWLGVIWSGGNTDYNTPFTSRLSINKD |
| | | NSKSQVFFKMNSLQSQDTAIYYCARALTYYDYEFAYWGQGTLVTVSA |
| | | ASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKV |
| | | DKKVEPKSCDKTHTCPPCPAPEFEGGPSVFLFPPKPKDTLMISRTPE |
| | | VTCVVVDVSHEDPEVKFNWYVDGVEVHNAKTKPREEQYQSTYRVVS |
| | | VLTVLHQDWLNGKEYKCKVSNKALPASIEKTISKAKGQPREPQVYTLP |
| | | PSREEMTKNQVSLTCLVKGFYPSDIAVEWESNGQPENNYKTTPPVLD |
| | | SDGSFFLYSKLTVDKSRWQQGNVFSCSVMHEALHNHYTQKSLSLSP |
| | | G |
| 121 | Complex-66-2^{nd} | LSCEGWAMNREQCRAGGGSSGGSISSGLLSGRSDQHGGGSQILLT |
| | polypeptide | QSPVILSVSPGERVSFSCRASQSIGTNIHWYQQRTNGSPRLLIKYASE |
| | without spacer | SISGIPSRFSGSGSGTDFTLSINSVESEDIADYYCQQNNNWPTTFGAG |
| | | TKLELKRTVAAPSVFIFPPSDEQLKSGTASVVCLLNNFYPREAKVQWK |
| | | VDNALQSGNSQESVTEQDSKDSTYSLSSTLTLSKADYEKHKVYACEV |
| | | THQGLSSPVTKSFNRGEC |
| 122 | CI106-1^{st} | GYLWGCEWNCGGITTGSSGGSGGSGGLSGRSDDHGGGSQTVVTQ |
| | polypeptide | EPSFSVSPGGTVTLTCRSSTGAVTTSNYANWVQQTPGQAPRGLIGG |
| | without spacer | TNKRAPGVPDRFSGSILGNKAALTITGAQADDESDYYCALWYSNLWV |
| | | FGGGTKLTVLGGGGSGGGGSGGGGSEVQLVESGGGLVQPGGSLKL |
| | | SCAASGFTFSTYAMNWVRQASGKGLEWVGRIRSKYNNYATYYADSV |
| | | KDRFTISRDDSKNTAYLQMNSLKTEDTAVYYCTRHGNFGNSYVSWF |
| | | AYWGQGTLVTVSSGGGGSQVQLKQSGPGLVQPSQSLSITCTVSGFS |
| | | LTNYGVHWVRQSPGKGLEWLGVIWSGGNTDYNTPFTSRLSINKDNS |
| | | KSQVFFKMNSLQSQDTAIYYCARALTYYDYEFAYWGQGTLVTVSAAS |
| | | TKGPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVDKK |
| | | VEPKSCDKTHTCPPCPAPELLGGPSVFLFPPKPKDTLMISRTPEVTCV |
| | | VVDVSHEDPEVKFNWYVDGVEVHNAKTKPCEEQYGSTYRCVSVLTV |
| | | LHQDWLNGKEYKCKVSNKALPAPIEKTISKAKGQPREPQVYTLPPSR |
| | | KEMTKNQVSLTCLVKGFYPSDIAVEWESNGQPENNYKTTPPVLKSDG |
| | | SFFLYSKLTVDKSRWQQGNVFSCSVMHEALHNHYTQKSLSLSPGK |
| 123 | Linker | GGGSSGGS |
| 124 | Linker | (GGGGS)n |
| 125 | Linker | GSSGGSGGSG |
| 126 | Complex-66 | CAAGGACAATCTGGCTCTGTGTCCACCACCTGTTGGTGGGACCCT |
| | Polynucleotide | CCATGCACACCTAATACCGGCAGCTCTGGTGGCTCTGGCGGAAG |
| | encoding 1^{st} | CGGAGGACTGTCTGGCAGATCCGATGATCACGGCGGAGGATCTG |
| | Polypeptide | AGGTGCAGCTGGTTGAATCTGGTGGCGGACTGGTTCAGCCTGGC |
| | without C-terminal | GGATCTCTGAAACTGAGCTGTGCCGCCAGCGGCTTCACCTTCAAC |
| | lysine | AAATACGCCATGAACTGGGTCCGACAGGCCCCTGGCAAAGGCCTT |
| | | GAATGGGTCGCCAGAATCAGAAGCAAGTACAACAACTATGCCACC |
| | | TACTACGCCGACAGCGTGAAGGACAGATTCACCATCAGCCGGGA |
| | | CGACAGCAAGAACACCGCCTACCTGCAGATGAACAACCTGAAAAC |
| | | CGAGGACACCGCCGTGTACTACTGTGTGCGGCACGGCAACTTCG |
| | | GCAACAGCTACATCAGCTACTGGGCCTATTGGGGCCAGGGCACA |
| | | CTGGTCACAGTTTCTAGTGGCGGAGGCGGATCTGGCGGCGGTGG |
| | | AAGTGGCGGCGGAGGTTCTCAAACAGTGGTCACCCAAGAGCCTA |
| | | GCCTGACCGTTTCTCCTGGCGGAACCGTGACACTGACATGCGGAT |
| | | CTTCTACAGGCGCCGTGACCAGCGGCAACTACCCTAATTGGGTGC |
| | | AGCAGAAGCCAGGCCAGGCTCCTAGAGGACTGATCGGCGGCACA |
| | | AAGTTTCTGGCTCCCGGAACACCAGCCAGATTCAGCGGTTCTCTG |
| | | CTCGGAGGAAAGGCCGCTCTGACACTTTCTGGCGTGCAGCCTGA |
| | | GGATGAGGCCGAGTACTATTGCGTGCTGTGGTACAGCAACAGATG |
| | | GGTGTTCGGCGGAGGCACCAAGCTGACAGTTCTTGGAGGTGGCG |
| | | GTAGCCAGGTCCAGCTGAAACAATCTGGACCCGGACTCGTGCAG |
| | | CCAAGCCAGAGCCTGTCTATCACCTGTACCGTGTCCGGCTTCAGC |
| | | CTGACCAATTACGGCGTGCACTGGGTTCGACAATCTCCCGGCAAG |
| | | GGACTCGAATGGCTGGGAGTGATTTGGAGCGGCGGCAACACCGA |
| | | CTACAACACCCCATTCACCAGCAGACTGAGCATCAACAAGGACAA |
| | | CAGCAAGTCCCAGGTGTTCTTCAAGATGAACTCCCTGCAGAGCCA |
| | | GGATACCGCCATCTATTACTGCGCTCGGGCCCTGACCTACTATGA |
| | | CTACGAGTTTGCCTACTGGGGACAGGGAACCCTCGTGACAGTGTC |
| | | TGCTGCTAGCACAAAGGGCCCTAGCGTTTTCCCACTGGCTCCCAG |
| | | CAGCAAGTCTACATCCGGTGGAACAGCCGCTCTGGGCTGCCTGG |
| | | TCAAGGATTACTTTCCCGAGCCAGTGACCGTGTCCTGGAATAGCG |
| | | GAGCACTGACATCTGGCGTGCACACATTTCCAGCCGTGCTGCAGT |
| | | CTAGCGGCCTGTACTCTCTGTCCAGCGTTGTGACAGTGCCCAGCA |
| | | GCTCTCTGGGCACCCAGACCTACATCTGCAATGTGAACCACAAGC |
| | | CTAGCAACACCAAGGTGGACAAGAAGGTGGAACCCAAGAGCTGC |
| | | GATAAGACACACACCTGTCCTCCATGTCCTGCTCCAGAGCTGCTC |
| | | GGAGGCCCTTCCGTGTTTCTGTTCCCTCCAAAGCCTAAGGACACC |
| | | CTGATGATCAGCAGAACCCCTGAAGTGACCTGCGTGGTGGTGGAT |
| | | GTGTCCCACGAGGATCCCGAAGTGAAGTTCAATTGGTACGTCGAC |
| | | GGCGTGGAAGTGCACAATGCCAAGACCAAGCCTTGCGAGGAACA |
| | | GTACGGCAGCACCTACAGATGCGTGTCCGTGCTGACAGTGCTGC |
| | | ACCAGGATTGGCTGAACGGCAAAGAGTACAAGTGCAAGGTGTCCA |
| | | ACAAGGCCCTGCCTGCTCCTATCGAGAAAACCATCAGCAAGGCCA |
| | | AGGGCCAGCCTAGAGAACCCCAGGTGTACACACTGCCTCCAAGC |
| | | CGGAAAGAGATGACCAAGAATCAGGTGTCCCTGACCTGCCTGGTC |
| | | AAGGGCTTCTACCCTTCCGATATCGCCGTGGAATGGGAGAGCAAT |
| | | GGACAGCCCGAGAACAACTACAAGACAACCCCTCCTGTGCTGAAG |
| | | TCCGACGGCTCATTCTTCCTGTACAGCAAGCTGACCGTGGACAAG |
| | | AGCAGATGGCAGCAGGGCAACGTGTTCAGCTGCAGCGTGATGCA |
| | | CGAGGCCCTGCACAACCACTACACCCAGAAGTCCCTGTCTCTGAG |
| | | CCCCGGC |
| 127 | Complex-66 1^{st} | QGQSGSVSTTCWWDPPCTPNTGSSGGSGGSGGLSGRSDDHGGGS |
| | Polypeptide with | EVQLVESGGGLVQPGGSLKLSCAASGFTFNKYAMNWVRQAPGKGL |
| | spacer and C- | EWVARIRSKYNNYATYYADSVKDRFTISRDDSKNTAYLQMNNLKTED |
| | terminal lysine | TAVYYCVRHGNFGNSYISYWAYWGQGTLVTVSSGGGGSGGGGSG |
| | | GGGSQTVVTQEPSLTVSPGGTVTLTCGSSTGAVTSGNYPNWVQQK |
| | | PGQAPRGLIGGTKFLAPGTPARFSGSLLGGKAALTLSGVQPEDEAEY |
| | | YCVLWYSNRWVFGGGTKLTVLGGGGSQVQLKQSGPGLVQPSQSLSI |
| | | TCTVSGFSLTNYGVHWVRQSPGKGLEWLGVIWSGGNTDYNTPFTSR |
| | | LSINKDNSKSQVFFKMNSLQSQDTAIYYCARALTYYDYEFAYWGQGT |
| | | LVTVSAASTKGPSVFPLAPSSKSTSGGTAALGCLVKDYFPEPVTVSW |
| | | NSGALTSGVHTFPAVLQSSGLYSLSSVVTVPSSSLGTQTYICNVNHKP |
| | | SNTKVDKKVEPKSCDKTHTCPPCPAPEFEGGPSVFLFPPKPKDTLMI |
| | | SRTPEVTCVVVDVSHEDPEVKFNWYVDGVEVHNAKTKPREEQYQST |
| | | YRVVSVLTVLHQDWLNGKEYKCKVSNKALPASIEKTISKAKGQPREP |
| | | QVYTLPPSREEMTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| 128 | Complex-66-1^{st} | VSTTCWWDPPCTPNTGSSGGSGGSGGLSGRSDDHGGGSEVQLVE |
| | polypeptide | SGGGLVQPGGSLKLSCAASGFTFNKYAMNWVRQAPGKGLEWVARI |
| | without spacer and | RSKYNNYATYYADSVKDRFTISRDDSKNTAYLQMNNLKTEDTAVYYC |
| | with C-terminal | VRHGNFGNSYISYWAYWGQGTLVTVSSGGGGGGGGSGGGGSQT |
| | lysine | VVTQEPSLTVSPGGTVTLTCGSSTGAVTSGNYPNWVQQKPGQAPR |
| | | GLIGGTKFLAPGTPARFSGSLLGGKAALTLSGVQPEDEAEYYCVLWY |
| | | SNRWVFGGGTKLTVLGGGGSQVQLKQSGPGLVQPSQSLSITCTVSG |
| | | FSLTNYGVHWVRQSPGKGLEWLGVIWSGGNTDYNTPFTSRLSINKD |
| | | NSKSQVFFKMNSLQSQDTAIYYCARALTYYDYEFAYWGQGTLVTVSA |
| | | ASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKV |
| | | DKKVEPKSCDKTHTCPPCPAPEFEGGPSVFLFPPKPKDTLMISRTPE |
| | | VTCVVVDVSHEDPEVKFNWYVDGVEVHNAKTKPREEQYQSTYRVVS |
| | | VLTVLHQDWLNGKEYKCKVSNKALPASIEKTISKAKGQPREPQVYTLP |
| | | PSREEMTKNQVSLTCLVKGFYPSDIAVEWESNGQPENNYKTTPPVLD |
| | | SDGSFFLYSKLTVDKSRWQQGNVFSCSVMHEALHNHYTQKSLSLSP |
| | | GK |
| 129 | CM | ALAHGLF |
| 130 | CM | APRSALAHGLF |
| 131 | CM | ISSGLLSGRSNI |
| 132 | CM | LSGRSNI |

The disclosure is not to be limited in scope by the aspects described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes.

Some aspects are within the following claims.

What is claimed is:

1. An activatable anti-EGFR, anti-CD3 heteromultimeric bispecific polypeptide complex (HBPC) comprising:
(a) a first polypeptide comprising (i) a first single-chain variable fragment (scFv) comprising a first heavy chain variable domain (VH1) and a first light chain variable domain (VL1) that together form a T-cell cluster of differentiation (CD3)-targeting domain that specifically binds a first CD3 polypeptide, (ii) a first masking moiety (MM1), (iii) a first cleavable moiety (CM1); and (iv) a second heavy chain variable domain (VH2) and (v) a first monomeric Fc domain (Fc1);
(b) a second polypeptide comprising (i) a second light chain variable domain (VL2), wherein VL2 and VH2 together form an EGFR-targeting domain that specifically binds a first EGFR, (ii) a second masking moiety (MM2), and (iii) a second cleavable moiety (CM2);
(c) a third polypeptide comprising (i) a second scFv comprising a third heavy chain variable domain (VH3) and a third light chain variable domain (VL3) that together form a CD3-targeting domain that specifically binds a second CD3 polypeptide, (ii) a third masking moiety (MM3), (iii) a third cleavable moiety (CM3); (vi) a fourth heavy chain variable domain (VH4), and (v) a second monomeric Fc domain (Fc2); and
(d) a fourth polypeptide comprising (i) a fourth light chain variable domain (VL4), wherein VL4 and VH4 together form an EGFR-targeting domain that specifically binds a second EGFR (VH4), (ii) a fourth masking moiety (MM4), and (iii) a fourth cleavable moiety (CM4),
wherein VH1 and VH3 each comprise
(i) a VH CDR1 comprising the amino acid sequence KYAMN (SEQ ID NO: 43),
(ii) a VH CDR2 comprising the amino acid sequence RIRSKYNNYATYYADSVKD (SEQ ID NO:44), and
(iii) a VH CDR3 comprising the amino acid sequence HGNFGNSYISYWAY (SEQ ID NO:45);
wherein VL1 and VH3 each comprise
(i) a VL CDR1 comprising the amino acid sequence SSTGAVTSGNYPNG (SEQ ID NO:40),
(ii) a VL CDR2 comprising the amino acid sequence GTKFLAP (SEQ ID NO: 41), and
(iii) a VL CDR3 comprising the amino acid sequence VLWYSNRWV (SEQ ID NO:42);
wherein VH2 and VH4 each comprise
(i) a VH CDR1 comprising the amino acid sequence NYGVH (SEQ ID NO: 37),
(ii) a VH CDR2 comprising the amino acid sequence VIWSGGNTDYNTPFTS (SEQ ID NO:38),
(iii) a VH CR3 comprising the amino acid sequence ALTYYDYEFAY (SEQ ID NO:39);
wherein VL2 and VL4 each comprise
(i) a VL CDR1 comprising the amino acid sequence RASQSIGTNIH (SEQ I D NO: 34),
(ii) a VL CDR2 comprising the amino acid sequence YASESIS (SEQ ID NO: 35),
(iii) a VL CDR3 comprising the amino acid sequence QQNNNWPTT.

2. The activatable HBPC of claim 1, wherein: (1) the first polypeptide comprises the amino acid sequence of SEQ ID NO:120, (2) the second polypeptide comprises the amino acid sequence of SEQ ID NO:121, (3) the third polypeptide comprises the amino acid sequence of SEQ ID NO: 120, and (4) the fourth polypeptide comprises the amino acid sequence of SEQ ID NO: 121.

3. The activatable bispecific polypeptide complex of claim 1, wherein (1) the first polypeptide comprises the amino acid sequence of SEQ ID NO:2, (2) the second polypeptide comprises the amino acid sequence of SEQ ID NO:16, (3) the third polypeptide comprises the amino acid sequence of SEQ ID NO:2, and (4) the second polypeptide comprises the amino acid sequence of SEQ ID NO:16.

4. The activatable bispecific polypeptide complex of claim 1, wherein the first and third polypeptides comprise the amino acid sequence of SEQ ID NO:128.

5. The activatable bispecific polypeptide complex of claim 3, wherein the first and third polypeptides comprise the amino acid sequence of SEQ ID NO: 127.

6. A pharmaceutical composition comprising the activatable bispecific polypeptide complex of any one of claims 1-5 and a pharmaceutically acceptable carrier.

7. A kit comprising the pharmaceutical composition of claim 6.

8. A nucleic acid comprising nucleotide sequences that encode the first polypeptide, the second polypeptide, the third polypeptide, and the fourth polypeptide of the activatable bispecific polypeptide complex of any one of claims 1-5.

9. A vector comprising the nucleic acid of claim 8.

10. A host cell comprising the vector of claim 9.

11. A method of producing an activatable anti-EGFR, anti-CD3 heteromultimeric bispecific polypeptide complex (HBPC) comprising: (a) culturing the host cell of claim 10 in a liquid culture medium under conditions sufficient to produce the activatable HBPC; and
(b) recovering the activatable HBPC.

12. A method of treating a disease in a subject comprising administering a therapeutically effective amount of the activatable bispecific polypeptide complex of any one of claims 1-5 or the pharmaceutical composition of claim 6 to the subject.

13. The method of claim 12, wherein the subject is a human.

14. The method of claim 12 or 13, wherein the disease is a cancer.

15. The activatable bispecific polypeptide complex of any one of claims 1-5 or the pharmaceutical composition of claim 6 for use in inhibiting tumor growth in a subject in need thereof.

16. Use of an activatable bispecific polypeptide complex according to any one of claims 1-5 or the pharmaceutical composition of claim 6 in the manufacture of a medicament for treating cancer.
